(19) United States
(12) Patent Application Publication
(10) Pub. No.: US 2007/0086998 A1
(43) Pub. Date: Apr. 19, 2007

(54) Therapeutic human anti-mhc class II antibodies and their uses

(75) Inventor: Zoltan Nagy, Wolfratshausen (DE)

Correspondence Address: FISH & NEAVE IP GROUP;ROPES & GRAY LLP, ONE INTERNATIONAL PLACE, BOSTON, MA, 02110-2624, US

(73) Assignee: GPC Biotech AG, Martinsried/Munich (DE)

(21) Appl. No.: 10/571,449

(22) PCT Filed: Sep. 9, 2004
(86) PCT No.: PCT/EP04/10075

§ 371 Date: Nov. 20, 2006

Related U.S. Application Data
(60) Provisional application No. 60/501,608, filed on Sep. 9, 2003.

(51) Int. Cl. A61K 39/395

(52) U.S. Cl. 424/144.1

(57) ABSTRACT

The instant invention relates to methods, compositions, uses related to the compositions and pharmaceutical packages for treating a disorder involving cells expressing MHC class II antigens using a combination of a human antibody-based antigen-binding domain that binds to a human Class II MHC molecule, and an antibody-based antigen-binding domain that binds to a cell surface receptor. Such disorders include cell proliferative disorders like lymphomas, leukemias, and certain solid tumors including melanomas, as well as disorders characterized by unwanted activation of immune cells like rheumatoid arthritis and multiple sclerosis.

### TECHNICAL FIELD

This invention relates to methods and compositions, and uses pertaining to these compositions, for the treatment of disorders involving cells expressing MHC class II antigens. Such disorders include cell proliferative disorders like lymphomas, leukemias, and certain solid tumors including melanomas, as well as disorders characterized by unwanted activation of immune cells like rheumatoid arthritis and multiple sclerosis.

### BACKGROUND OF THE INVENTION

Therapeutic Need

In the United States, more than 500,000 people die of cancer each year, which corresponds to more than 1,500 cancer deaths per day. Currently, about 10 million Americans with a history of cancer are living, with more than 1,300,000 new cases of cancer expected to be diagnosed in the United States in 2004 alone, and yet the 5-year relative survival rate for all cancers combined is only around 60%.

Certain particularly prevalent cancers have poor prognosis and poor expectation of survival even if diagnosed and treated at an early stage of the disease. These cancers can include those associated with tumors cells that express MHC class II antigens such as lymphomas (for example, Non-Hodgkin's Lymphoma), leukemias, and certain solid tumours including melanomas.

Lymphoma is the most commonly occurring blood cancer. Approximately 500,000 people in the United States are living with lymphoma, which causes about 27,600 deaths each year. Non-Hodgkin's Lymphoma (NHL) alone is the fifth most common of all cancers in the United States, with a person's risk of developing NHL during their lifetime at about 1 in 50. The main types of treatment of NHL are radiation therapy, chemotherapy, immunotherapy and bone marrow and peripheral blood transplants.

One of the most commonly used chemotherapeutic treatments for NHL is CHOP, a combination treatment comprising Cyclophosphamide, adramycin (doxorubicin/Hydroxydoxorubicin), vincristine (Oncovine) and Prednisone. However, low complete response rates and high relapse rates are common, particularly in elderly patients and in patients with aggressive forms of NHL. Furthermore, CHOP treatment often has unpleasant side effects including permanent sterility, a drop in blood counts, left ventricular dysfunction, peripheral neuropathy, an elevated risk of second primary cancers, hair loss, a sore mouth, nausea, vomiting, loss of appetite and fatigue. Other treatment options for NHL include: (i) chlorambucil, (ii) fludarabine, (iii) COP (as CHOP, but not using adramycin), (iv) PMitCEBO (a combination therapy comprising mitoxantrone or mitozantrone, cyclophosphamide, etoposide, bleomycin, vincristine and prednisolone), (v) DHAP (a combination therapy comprising cytarabine, cisplatin and dexamethasone) and (vi) ESHAP (a combination therapy comprising (etoposide, methylprednisolone, cytarabine and cisplatin). However, each of these treatment options shows limited efficacy and is associated with various unpleasant side effects.

The most commonly used class of agents used in immunotherapy of NHL is monoclonal antibodies. Among them, the most prominent is rituximab (Rituxan®, MabThera®), a monoclonal antibody targeting CD20. Rituximab is used in the treatment of NHL, either alone or in combination with other chemotherapeutic agents (Curr Pharm Biotechnol (2001), Vol. 2, p. 301-311; Prog Oncol (2001), p. 204-227; Press Release of Protein Design Labs from Oct. 29, 2001; Hematology (Am Soc Hematol Educ Program) (2001) p. 221-40) Rituximab was the first monoclonal antibody approved by the FDA for the treatment of a cancer. However, it is not effective for treating certain subtypes of NHL. Over a number of studies, the overall response rate (including partial and complete responses) in patients receiving rituximab was reported to vary by as much as 30% and 70%, which means that still many patients die after rituximab treatment.

More than 50,000 cases of melanoma are diagnosed in the United States each year and 7,800 deaths were attributed to melanoma in 2001. A person's risk of developing melanoma during their lifetime is about 1 in 71. The first treatment of melanoma is usually the removal of the melanoma by surgical excision. Surgery may be combined or followed up (adjuvant therapy) with chemotherapy or immunotherapy (Annals Pharmacother (1999) Vol. 33, p. 730-738; ASCO 2001 Annual Meeting, Abstract 1181, Lancet Oncol (2003), Vol. 4, p. 748-759). The most commonly used drug in chemotherapy is dacarbazine, which is often used in combination with other drugs such as carmustine, cisplatin and tamoxifen. However, most chemotherapeutic agents are insufficiently active against melanoma to cure more than a small minority of patents. For example, the response rate of melanoma patients treated with dacarbazine is only between 20-30%.

Despite substantial efforts and investment made by the biopharmaceutical industry to identify and develop new drug candidates, drugs and treatment methods for disorders associated with cells that express MHC II molecules, including lymphomas such as Non-Hodgkin's Lymphoma, leukemias, certain solid tumours including melanomas, and rheumatoid arthritis and multiple sclerosis, there still remains a need to provide new therapeutic opportunities to develop treatments for such disorders. In particular, new therapies for treatment of cancers such as NHL and melanoma are urgently needed. Such methods are provided herein.

Major Histocompatibility Complex (MHC)

Every mammalian species that has been studied to date carries a cluster of genes coding for the so-called major histocompatibility complex (MHC). This tightly linked cluster of genes code for surface antigens, which play a central role in the development of both humoral and cell-mediated immune responses. In humans the products coded for by the MHC are referred to as Human Leukocyte Antigens or HLA. The MHC-genes are organized into regions encoding three classes of molecules, class I to III.

Class I MHC molecules are 45 kD transmembrane glycoproteins, noncovalently associated with another glycoprotein, the 12 kD beta-2 microglobulin (Brown et al., 1993). The latter is not inserted into the cell membrane, and is encoded outside the MHC. Human class I molecules are of three different isotypes, termed HLA-A, -B, and -C, encoded in separate loci. The tissue expression of class I molecules is ubiquitous and codominant. MHC class I molecules present peptide antigens necessary for the activation of cytotoxic T-cells.

Class II MHC molecules are noncovalently associated heterodimers of two transmembrane glycoproteins, the 35 kD α chain and the 28 kD β chain (Brown et al., 1993). In humans, class II molecules occur as three different isotypes, termed human leukocyte antigen DR (HLA-DR), HLA-DP and HLA-DQ. Polymorphism in DR is restricted to the 0 chain, whereas both chains are polymorphic in the DP and DQ isotypes. Class II molecules are expressed codominantly, but in contrast to class I, exhibit a restricted tissue distribution: they are present only on the surface of cells of the immune system, for example dendritic cells, macrophages, B lymphocytes, and activated T lymphocytes. They are also expressed on human adrenocortical cells in the zona reticularis of normal adrenal glands and on granulosa-lutein cells in corpora lutea of normal ovaries (Kahoury et al., 1990). Their major biological role is to bind antigenic peptides and present them on the surface of antigen presenting cells (APC) for recognition by CD4 helper T (Th) lymphocytes (Babbitt et al., 1985). MHC class II molecules can also be expressed on the surface of non-immune system cells, for example, cells that express MHC class II molecules during a pathological inflammatory response. These cells can include synovial cells, endothelial cells, thyroid stromal cells and glial cells (Cell (2002) Vol. 109 Rev. Suppl., P.S21-S33; Microbes & Infection (1999) Vol. 1, p. 893-902). In particular, cells associated with certain solid tumours express MHC class II molecules, such as melanoma cells (Cancer Biol (1991) Vol. 2, p 35-45; J. Immunol. (2001) Vol. 167, p. 98-106).

Class II MHC molecules are also associated with immune responses, but encode somewhat different products. These include a number of soluble serum proteins, enzymes and proteins like tumor necrosis factor or steroid 21-hydroxylase enzymes. In humans, class III molecules occur as three different isotypes, termed Ca, C2 and Bf (Kuby, 1994).

Since Th cell activation is a crucial event of the initiation of virtually all immune responses and is mediated through class II molecules, class II MHC offers itself as a target for immunomodulation (Baxevanis et al., 1980; Rosenbaum et al., 1981; Adorini et al., 1988). Besides peptide presentation, class II molecules can transduce various signals that influence the physiology of APC. Such signals arise by the interaction of multiple class II molecules with an antibody or with the antigen receptor of Th cells (Vidovic et al., 1995a; Vidovic et al., 1995b), and can induce B cell activation and immunoglobulin secretion (Cambier et al., 1991; Palacios et al., 1983), cytokine production by monocytes (Palacios, 1985) as well as the up-regulation of co-stimulatory (Nabavi et al., 1992) and cell adhesion molecules (Mourad et al., 1990).

There is also a set of observations suggesting that class II ligation, under certain conditions, can lead to cell growth arrest or be cytotoxic. Ligation under these conditions is the interaction of a polypeptide with a class II MHC molecule. There is substantial contradiction about the latter effects and their possible mechanisms. Certain authors claim that formation of a complex of class II molecules on B cells leads to growth inhibition (Vaickus et al., 1989; Kabelitz et al., 1989), whereas according to others class II complex formation results in cell death (Vidovic et al., 1995a; Newell et al., 1993; Truman et al., 1994; Truman et al., 1997; Drenou et al., 1999). In certain experimental systems, the phenomenon was observed with resting B cells only (Newell et al., 1993), or in other systems with activated B cells only (Vidovic et al., 1995a; Truman et al., 1994). A general review of MHC class II mediated cell growth arrest or cytotoxicity is provided by Nagy and Mooney (J Mol Med (2003), Vol. 81, p. 757-765).

Based on these observations, anti-class II monoclonal antibodies (mAbs) have been envisaged for a number of years as therapeutic candidates. Indeed, this proposal has been supported by the beneficial effect of mouse-derived anti-class II mAbs in a series of animal disease models (Waldor et al., 1983; Jonker et al., 1988; Stevens et al., 1990; Smith et al., 1994; Vidovic & Torral, 1998; Vidovic & Laus, 2000).

Despite these early supporting data, and except for those described in US 2003/0032782 and Nat Medicine (200) Vol. 8, p. 801-807), to date no human anti-MHC class II mAb has been described that displays the desired cytotoxic and other biological properties which may include affinity, efficiency of killing and selectivity. Indeed, despite the relative ease by which mouse-derived mAbs may be derived, work using mouse-derived mAbs has demonstrated the difficulty of obtaining an antibody with the desired biological properties. For example, significant and not fully understood differences were observed in the T cell inhibitory capacity of different murine anti-class II mAbs (Naquet et al., 1983). Furthermore, the application of certain mouse-derived mAbs in vivo was associated with unexpected side effects, sometimes resulting in death of laboratory primates (Billing et al., 1983; Jonker et al., 1991).

It is generally accepted that mouse-derived mAbs (including chimeric and so-called “humanized” mAbs) carry an increased risk of generating an adverse immune response (Human anti-murine antibody—HAMA) in patients compared to treatment with a human mAb (for example, Vose et al, 2000; Kashmiri et al., 2001). This risk is potentially increased when treating chronic diseases such as rheumatoid arthritis or multiple sclerosis with any mouse-derived mAb or where regular treatment may be required, for example in the treatment of certain cancers; prolonged exposure of the human immune system to a on-human molecule often leads to the development of an adverse immune reaction. Furthermore, it has proven very difficult to obtain mouse-derived antibodies with the desired specificity or affinity to the desired antigen (Pichla et al. 1997). Such observation may significantly reduce the overall therapeutic effect or advantage provided by mouse-derived mAbs. Examples of disadvantages for mouse-derived mAbs may include the following. First, mouse-derived mAbs may be limited in the medical conditions or length of treatment for a condition for which they are appropriate. Second, the dose rate for mouse-derived mAbs may need to be relatively high in order to compensate for a relatively low affinity or therapeutic effect, hence making the dose not only more severe but potentially more immunogenic and perhaps dangerous. Third, such restrictions in suitable treatment regimes and high-dose rates requiring high production amounts may significantly add to the cost of treatment and could mean that such a mouse-derived mAb be uneconomical to develop as a commercial therapeutic. Finally, even if a mouse mAb could be identified that displayed the desired specificity or affinity, often these desired features are detrimentally affected during the “humanization” or “chimerization” procedures necessary to reduce immunogenic potential (Slavin-Chiorini et al., 1997). Once a mouse-derived mAb has been “humanized” or chimerized, then it is very difficult to optimize its specificity or affinity.

The art has sought over a number of years for human anti-MHC class II mAbs that show biological properties suitable for use in a pharmaceutical composition for the treatment of humans. Workers in the field have practiced the process steps of first identifying a mouse-derived mAb, and then modifying the structure of this mAb with the aim of improving immunotolerance of this non-human molecule for human patients (for further details, see Jones et al., 1986; Riechmann et al., 1988; Presta, 1992). This modification is typically made using so-called “humanization” procedures or by fabricating a human-mouse chimeric mAb. Examples of other antibodies that bind MHC class II antigen and cause or lead to killing of cells expressing such antigen include Danton/DN1924 (Dendreon) such as described in U.S. Pat. No. 6,416,958, “HD” antibodies such as HD4 and HD8 (Kirin), as described in WO 03/033538, and 1D10 and Hu1D10 (Remitogen®, apolizumab; Protein Design Labs) as described by Kostelny et al (Int J Cancer 93:556-65). Other workers have attempted to identify human antibodies that bind to human antigens having desired properties within natural repertoires of human antibody diversity. For example, by exploring the fetal-tolerance mechanism in pregnant women (Bonagura et al., 1987) or by panning libraries of natural-diversities of antibodies (Stausbøl-Grøn et al., 1996; Winter et al., 1994). However, except for those described in US 2003/0032782 and Nat Medicine (200) Vol. 8, p. 801-807, to date no human anti-MHC class II mAb has been described that displays appropriate biological properties of one or more of cytotoxicity, selectivity, specificity and affinity.

For the therapeutic purposes of the instant invention, a polypeptide reacting with most or at least many of the common allelic forms of a human class II MHC molecule would be desirable—e.g., to enable its use in diverse patient populations. Moreover, the candidate polypeptide should be cytotoxic to a wide range of lymphoid tumors, and preferably is cytotoxic by way of a mechanism common to such a range of tumor cells. To allow for a wide range of possible applications, the polypeptide desired should mediate its cytotoxic effect without the dependence on further components of the immune system. For therapeutic purposes, most patients receive for the treatment of, e.g. cancer, standard chemo- or radiotherapy. Most of these treatments leave the patient immunocompromised. Any additional treatment that relies on an intact immune system is therefore likely to fail. The underlying problem is further demonstrated in humans who suffer from a disease that destroys the immune system, e.g. HIV. Opportunistic infections and malignant transformations are able to escape the immune-surveillance and cause further complications.

### SUMMARY OF THE INVENTION

This present invention provides opportunities for new therapeutic methods, compositions and uses of a variety of antibody-based drug-candidates/drugs, where following the disclosure herein, such antibody-based drug-candidates/drugs can be suitable for further pre-clinical or clinical research and development towards the treatment of a variety of disorders, particularly lymphomas, leukemias, certain solid tumours including melanomas, but also including rheumatoid arthritis and multiple sclerosis. The further development of such new therapeutic opportunities provided by the present invention can result in one or more effective therapies, and marketed drugs, for particularly debilitating diseases including haematologial tumors such as Non-Hodgkin's Lymphoma (NHL), melanoma and degenerative disorders such as multiple sclerosis (MS).

The present invention is based, at least in part, on Applicants' two novel discoveries. First, Applicants discovered that a human antibody that binds to a human class II MEC molecule such as 1D09C3 mAb (also called “MS-GPC-8-27-41”), and an antibody that binds to a cell surface receptor, such as rituximab, show synergistic effect in treating lymphoid tumors, such as NHL (Example 23 and FIG. 18). Second, Applicants discovered that a human antibody, such as 1D09C3 mAb, alone can also induce cell death in non-lympoid solid tumors, as evidenced by killing of HLA-DR+ melanoma cells in vitro (Example 24 and FIG. 20).

Thus, one aspect of the present invention provides methods for treating a disorder comprising administering to an individual in need thereof a first polypeptide comprising an antibody-based antigen-binding domain that binds to a human class II MHC molecule, and a second polypeptide comprising an antibody-based antigen-binding domain that binds to a cell surface receptor. In particular embodiments, the “individual in need thereof” is an animal, such as a human. In certain embodiments, the first polypeptide comprises a human antibody-based antigen-binding domain that binds to a human class II MHC molecule. In certain embodiments, the first and/or the second polypeptides are formulated in a pharmaceutical preparation. In certain further embodiments, the first and the second polypeptide formulated in a pharmaceutical preparation are administered through a conjoined administration. For example, the first polypeptide and the second polypeptide may be administered either concurrently or sequentially. In one embodiment, the sequential administering of the first and the second polypeptide is within 24 hours of each other. Alternatively, the sequential administering of the first and the second polypeptide is within 3 days of each other, within 7 days of each other, or within 14 days of each other. For concurrent administration, the first and the second polypeptide may be administered as one single or as two separate pharmaceutically acceptable compositions.

Another aspect of the present invention provides methods for treating a solid tumor. The methods comprise administering to an individual in need thereof a first polypeptide comprising an antibody-based antigen-binding domain that binds to a human class II MHC molecule. In particular embodiments, the “individual in need thereof” is an animal, such as a human. In certain embodiments, the first polypeptide comprises a human antibody-based antigen-binding domain that binds to a human class II MHC molecule.

The forgoing methods, together with the other aspects of the invention including the further methods, uses, compositions, compositions for the uses described and pharmaceutical packs/compositions described herein, can be further characterized by one or more additional feature or features. These features include the first polypeptide, the second polypeptide, the disorder or cell type, and also the therapeutic schedule. As will be apparent to a person skilled it the art after the disclosure herein, any aspect of the invention may be further characterized by one, or more, or any combination of features used to further characterize another aspect of the invention. Hence, any combination of features described or claimed herein is encompassed within the scope of the invention for all aspects of the invention.

The first polypeptide may be a human antibody that binds to a human class II MHC molecule. Preferably, the antibody is a human monoclonal antibody. The monoclonal antibody may bind to any of the three isotypes of the class II MHC molecules, namely, HLA-DR, HLA-DP and HLA-DQ. In one embodiment, the first polypeptide comprises an antibody-based antigen-binding domain selected from: MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPC-8-27-10, MS-GPC-8-27-41. The first polypeptide may also be a variant or modified version of one of the above listed polypeptides.

In certain preferred embodiments, the human antibody-based antigen-binding domain that binds to a human class II MHC molecule is part of a multivalent polypeptide, such as one including at least a F(ab′)₂ antibody fragment or a mini-antibody fragment.

In certain preferred embodiments, the human antibody-based antigen-binding domain that binds to a human class II MHC molecule is part of a multivalent polypeptide comprising at least two monovalent antibody fragments selected from Fv, scFv, dsFv and Fab fragments, and further comprises a cross-linking moiety or moieties.

In certain preferred embodiments, the human antibody-based antigen-binding domain that binds to a human class II MHC molecule is part of a multivalent polypeptide comprising at least one full antibody selected from the antibodies of classes IgG₁, 2a, 2b, 3, 4, IgA, and IgM.

In certain preferred embodiments, the human antibody-based antigen-binding domain that binds to a human class II MHC molecule is part of a multivalent polypeptide is formed prior to binding to said cell.

In certain preferred embodiments, the human antibody-based antigen-binding domain that binds to a human class II MHC molecule is part of a multivalent polypeptide is formed after binding to said cell.

In certain preferred embodiments, the antibody-based antigen binding domains of the first polypeptide that binds to a human class II MHC molecule bind to one or more HLA-DR types selected from the group consisting of DR1-0101, DR2-15021, DR3-0301, DR4Dw4-0401, DR4Dw10-0402, DR4Dw14-0404, DR6-1302, DR6-1401, DR8-8031, DR9-9012, DRW53-B4*0101 and DRW52-B3*0101. In preferred embodiments, the antibody-based antigen binding domains of the first polypeptide provide broad-DR reactivity, that is, the antigen-binding domain(s) of a given composition binds to epitopes on at least 5 different of said HLA-DR types. In certain embodiments, the antigen binding domain(s) of a polypeptide(s) of the first polypeptide binds to a plurality of HLA-DR types as to bind to HLA-DR expressing cells for at least 60 percent of the human population, more preferably at least 75 percent, and even more preferably 85 percent of the human population.

In certain embodiments, the human antibody-based antigen binding domains of the first polypeptide that binds to a human class II MHC molecule include a combination of a VH domain and a VL domain, wherein said combination is found in one of the clones taken from the list of MS-GPC-1, MS-GPC-6, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-645, MS-GPC-8-6-13, MS-GPC-8-6-47, MS-GPC-8-10-57, MS-GPC-8-27-7, MS-GPC-8-27-10 and MS-GPC-8-27-41.

In certain embodiments, the human antibody-based antigen binding domains of the first polypeptide that binds to a human class II MHC molecule include a combination of HuCAL VH2 and HuCAL Vλ1, wherein the VH CDR3, VL CDR1 And VL CDR3 is found in one of the clones taken from the list of MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-647, MS-GPC-8-10-57, MS-GPC-8-27-7, MS-GPC-8-27-10 and MS-GPC-8-27-41.

In certain embodiments, the antigen-binding domains which binds to a human class II MHC molecule includes a combination of HuCAL VH2 and HuCAL Vλ1, wherein the VH CDR3 sequence is taken from the consensus CDR3 sequence:

| | |
|---|---|
| XXXXRGXFDX | (SEQ ID No. 1) |

wherein each X independently represents any amino acid residue; and/or,

wherein the VL CDR3 sequence is taken from the consensus CDR3 sequence:

| | |
|---|---|
| QSYDXXXX | (SEQ ID No. 2) |

wherein each X independently represents any amino acid residue. For instance, the VH CDR3 sequence can be SPRYRGAFDY (SEQ ID No. 3) and/or the VL CDR3 sequence can be QSYDLIRH (SEQ ID No. 4) or QSYDMNVH (SEQ ID No. 5).

In certain embodiments, the antigen-binding domains of the subject human antigen-binding domain binds to a human class II MHC molecule competes for antigen binding with an antibody including a combination of HuCAL VH2 and HuCAL Vλ1, wherein the VH CDR3 sequence is taken from the consensus CDR3 sequence:

| | |
|---|---|
| XXXXRGXFDX | (SEQ ID No. 1) |

each X independently represents any amino acid residue; and/or,

the VL CDR3 sequence is taken from the consensus CDR3 sequence:

| | |
|---|---|
| QSYDXXXX | (SEQ D No. 2) |

each X independently represents any amino acid residue. For instance, the VH CDR3 sequence of the antibody can be SPRYRGAFDY (SEQ ID No. 3) and/or the VL CDR3 sequence of the antibody can be QSYDLIRH (SEQ ID No. 4) or QSYDMNVH (SEQ ID No. 5).

In certain preferred embodiments, the human antibody-based antigen-binding domain which binds to a human class II MHC molecule includes a VL CDR1 sequence represented in the general formula:

| | |
|---|---|
| SGSXXNIGXNYVX | (SEQ ID No. 6) |

wherein each X independently represents any amino acid residue. For instance, the CDR1 sequence is SGSESNIGNNYVQ (SEQ ID No. 7).

In preferred embodiments, the first polypeptide, when a multivalent polypeptide includes at least two human antibody-based antigen-binding domains that bind human MHC class II, causes or leads to the killing of cells that express human class II MHC molecule by a mechanism that involves an innate pre-programmed process of said cell. In another preferred embodiment, said first polypeptide is further characterised in that treating or contacting cells expressing human class II MHC molecules with a multivalent first polypeptide having two or more of said antigen binding domains causes or leads to killing of said cells in a manner where neither cytotoxic entities nor immunological mechanisms are needed for said killing. For instance, said multivalent polypeptide can kill such cells in non-apoptotic mechanism. Killing by the subject compositions can be dependent on the action of non-caspase proteases, and/or killing which cannot be inhibited by zVAD-fmk or zDEVD-fmk. Appropriate methods to test the cytotoxic properties, characteristics or mechanisms of suitable polypeptides are described herein, such as examples 8 to 13, 15 and 24.

In certain further embodiments, the human monoclonal antibody of the first polypeptide is an IgG antibody obtainable by cloning into an immunoglobulin expression system an antigen-binding domain which includes a combination of a VH and a VL domain, wherein said combination is found in one of the clones MS-GPC-8-6-13, MS-GPC-8-10-57 or MS-GPC-8-27-41. For example, such a human IgG antibody can be or is obtained or generated according to a method such as described in example 5. In certain embodiments, the IgG antibody is an IgG4 antibody.

In certain embodiments, the first polypeptide is a multivalent polypeptide comprising a plurality of human antibody-based antigen-binding domains with binding specificity for human HLA-DR. Treating or contacting cells expressing HLA-DR with the multivalent polypeptide causes or leads to killing of the cell in a manner where neither cytotoxic entities nor immunological mechanisms are needed for killing. In other embodiments, treating or contacting cells expressing MHC class II with at least the first polypeptide, when a multivalent polypeptide, kills or inhibits the growth of such cell. In certain preferred embodiments, the antigen-binding domains individually bind to the human HLA-DR with a K_{d} of 1 μM, 100 nM, 10 nM or even 1 nM or less. In certain preferred embodiments, the multivalent polypeptide has an EC₅₀ of 100 nM, 10 nM or even 1 nM or less for killing activated lymphoid cells, transformed cells and/or lymphoid tumor cells.

In certain preferred embodiments, the first polypeptide can be characterized as including multivalent polypeptides having an EC₅₀ for killing transformed cells at least 5-fold lower than the EC₅₀ for killing normal cells, and even more preferably at least 10-fold, 100-fold and even 1000-fold less than for killing normal cells.

In certain preferred embodiments, the first polypeptide can be characterized as including multivalent polypeptides having an EC₅₀ for killing activated cells at least 5-fold lower than the EC₅₀ for killing unactivated cells, and even more preferably at least 10-folded, 100-fold and even 1000-fold less than for killing unactivated cells.

In certain preferred embodiments, the first polypeptide are characterized as including multivalent polypeptides having an EC₅₀ of 50 nM or less for killing transformed cells, and even more preferably an EC₅₀ of less than 10 nM, 1 nM and even 0.1 nM. In certain embodiments, the subject multivalent polypeptides have an EC₅₀ for killing activated lymphoid cells, transformed cells and/or lymphoid tumor cells of 100 nM, 10 nM or even 1 nM or less.

In certain embodiments, the first polypeptide can include multivalent polypeptides that selectively kill activated lymphoid cells. For example, such multivalent forms of the subject compositions can be used to kill activated lymphoid cells are lymphoid tumor cells representing a disease selected from B cell non-Hodgkin lymphoma, B cell lymphoma, B cell acute lymphoid leukemia, Burkitt lymphoma, Hodgkin lymphoma, hairy cell leukemia, acute myeloid leukemia, T cell lymphoma, T cell non-Hodgkin lymphoma, chronic myeloid leukemia, chronic lymphoid leukemia, and multiple myeloid leukemia.

According to a preferred embodiment, at least one polypeptide is directed to a lymphoid cell or a non-lymphoid cell that expresses MHC class II molecules. The latter type of cells occur for example at pathological sites of inflammation and/or autoimmune diseases, e.g. synovial cells, endothelial cells, thyroid stromal cells and glial cells, or it may also comprise genetically altered cells capable of expressing MHC class II molecules.

Preferably, at least one polypeptide is directed to lymphoid tumor cells. More preferred are lymphoid tumor cells that represent a disease selected from B cell non-Hodgkin lymphoma, B cell lymphoma, B cell acute lymphoid leukemia, Burkitt lymphoma, Hodgkin lymphoma, hairy cell leukemia, acute myeloid leukemia and B cell precursor leukemia. Most preferred are lymphoid tumor cells from a cell line taken from the list of GRANTA-519, PRIESS, KARPAS422, DOHH-2, MHH-CALL-4, MN-60, BJAB, L428, BONNA-12, EOL-1, MHH-PREB-1 and MHH-CALL-2 cell lines.

In a preferred embodiment, the first polypeptide comprising a human antibody-based antigen-binding domain which binds to a human class II MHC molecule induces a killing mechanism which is dependent on the action of proteases other than caspases, e.g., is a caspase-independent mechanism. In a further embodiment the multivalent composition which binds to a human class II MHC molecule comprises at least one full antibody which is selected from classes IgG₁, 2a, 2b, 3, 4, IgA, and IgM. In a further embodiment the multivalent composition which binds to a human class II MHC molecule comprises at least one of a F(ab′)₂ antibody fragment or mini-antibody fragment.

In a preferred embodiment, the multivalent composition which binds to a human class II MHC molecule comprises at least two monovalent antibody fragments selected from Fv, scFv, dsFv and Fab fragments, and further comprises a cross-linking moiety or moieties.

In a further preferred embodiment, the antibody-based antigen binding domains of the first polypeptide that binds to a human class II MHC molecule is modified compared to a parental antigen-binding domain disclosed in the present invention by addition, deletion and/or substitution of amino acid residues, while maintaining the properties according to the present invention, or improving one or more of said properties, of said parental antigen-binding domain. The following paragraphs described the terms ‘modified’ and ‘modification’ as used herein. This includes, but is not limited to, the modification of a nucleic acid sequence encoding a parental antigen-binding domain for cloning purposes, the modification of CDR regions in order to improve or modify antigen-binding affinity and/or specificity, including the exchange of one or more CDR sequences of a parental antigen-binding domain by corresponding CDR sequences from one or more different antigen-binding domains, and the addition of peptide sequences for detection and/or purification purposes. Modifications of a nucleic acid sequence, such as single nucleotide substitutions, may also occur as an artefact during cloning, propagation of cultures or as a result of other associated mutagenic events. Such modifications, while maintaining the properties according to the present invention, or improving one or more of said properties, are within the scope of the present invention. It is well within the scope of one of ordinary skill in the art to identify positions in a given parental antigen-binding domain where an addition, deletion and/or substitution should occur, to design and pursue the approach to achieve said addition, deletion and/or substitution, and to test or assay whether the modified antigen-binding domain has maintained the properties of, or exhibits one or more improved properties compared to, the parental antigen-binding domain. Furthermore, one of ordinary skill would be able to design approaches where collections or libraries of modified antigen-binding domains are designed, constructed and screened to identify one or more modified antigen-binding domain which have maintained the properties, or exhibit one or more improved properties compared to the parental antigen-binding domain. In one example, the third amino acid residue of a HuCAL VH domain comprised in any antigen-binding domain of the present invention, which is either E or Q depending on the expression construct, may be exchanged by Q or E, respectively. The same applies to the first amino acid residue of a HuCAL VH domain. Preferred regions to optimize an antigen-binding domain by designing, constructing and screening collections or libraries of modified antigen-binding domains according to the present invention comprise the CDR regions, and most preferably CDR3 of VH and VL, CDR1 of VL and CDR2 of VH domains.

Biologicals, such as antibodies, are susceptible to modifications which may arise during (cotranslationally) and/or after (post-translationally) translation. Such modification include, but are not limited to, glycosylation, acylation, methylation, phosphorylation, sulfation, prenylation, vitamin C-dependent modifications and vitamin K-dependent modifications. Another form of post-translational modification is cleavage of the produced polypeptide. While such cleavage may have functional aspects (i.e. the removal of the initiation methionine or the activation of proproteins), such cleavage may also occur in non-functional regions of a protein, for example at the C-terminus. In one example, the last amino acid residue of the heavy chain of an antibody comprising an antigen-binding domain of the present invention is cleaved. This amino acid residues may be a lysine residue. An amino acid substitution may also occur in the constant heavy or the constant light chain of an antibody. By way of non-limiting example, at position 150 of both light chains (Kabat numbering), there might be either a alanine or a glycine residue. Such modifications are within the scope of the present invention, while maintaining the properties according to the present invention, or improving one or more of said properties.

In particular aspects of the invention, the first polypeptide used in the methods, compositions or uses described herein is not a humanized or chimeric antibody. In alternative aspects of the invention, the first polypeptide used is one that comprises an antibody-based antigen-binding domain of human composition. In yet other aspects of the invention, the first polypeptide used is Danton/DN1924/DN1921 (Dendreon) such as described in U.S. Pat. No. 6,416,958, or an “HD” antibody such as HD4 or HD8 (Kirin) as described in WO 03/033538.

In certain embodiments the present invention provides compositions, methods or uses that include a first polypeptide comprising an antibody-based antigen-binding domain that binds to human HLA-DR with a K_{d} of 1 μM, 100 nM, 110 nM or even 1 nM or less, the antigen-binding domain being isolated by a method which includes isolation of human VL and VH domains from a recombinant antibody library by ability to bind to at least one epitope of human HLA-DR. Treating a cell expressing HLA-DR with such a multivalent polypeptide having two or more of the antigen binding domains causes or leads to killing of the cells in a manner where neither cytotoxic entities nor immunological mechanisms are needed for killing. In certain embodiments, the method for isolating the antigen-binding domain includes the further steps of: a) generating a library of variants of at least one of the CDR1, CDR2 and CDR3 sequences of one or both of the VL and VH domains, and, b) isolation of VL and VH domains from the library of variants by ability to bind to human HLA-DR with a K_{d} of 1 μM or less.

A subject first polypeptide, when multivalent polypeptide, can be capable of causing cell death of activated cells, preferably lymphoid tumor cells without requiring any further additional measures such as chemotherapy. Further, said multivalent polypeptide can have the capability of binding to at least one epitope on the target antigen, however, several epitope binding sites might be combined in one molecule. Preferably, the multivalent polypeptide shows at least 5-fold, or more preferably 10-fold higher killing activity against activated cells compared to non-activated cells. This higher activity on activated cells can be expressed as the at least 5-fold lower EC₅₀ value on activated versus non-activated cells or as the higher percentage of killing of activated cells versus non-activated cells when using the same concentration of protein. Under the latter alternative, the multivalent polypeptide at a given polypeptide concentration kills at least 50%, preferably at least 80%, of activated cells, whereas the same concentration of a multivalent polypeptide under the same incubation conditions kills less than 15%, preferably less than 10% of the non-activated cells. The assay conditions for determining the EC₅₀ value and the percentage killing activity are described below.

The second polypeptide of the methods, composition or uses may comprise an antibody-based antigen-binding domain that binds to a cell surface receptor. Preferably the second polypeptide binds to a cell surface receptor on a lymphocyte, such as, for example, a cell surface receptor on a B-cell. Alternatively, the second polypeptide binds to a cell surface receptor on a cell derived or included in a solid tumor, such as melanoma. The term “cell surface receptor”, as used herein, refers to a cell surface receptor, as well as co-receptors and other molecules associated with receptors and/or co-receptors. Non-limiting examples of such cell surface receptors are CD4, ICAMs, CD19, CD20, CD8, CD11a, CD11b, CD28, CD18, CD45, CD71, T cell receptor, B7, CD40, CD23, CD40L, CD23, CD22, CD35, CD18, CD80, CD32, CD52, CD33, Her-2/Neu, EGFR, PDGFR, Ep-CAM (EGP-2, GA 733-2), VEGF, CD37, and MHC class II molecules, such as HLA-DP, HLA-DQ and HLA-DR. Such cell surface receptors are well known to a skilled artisan (see e.g. I. Roitt, J. Brostoff & D. Male, Immunology (Mosby, 2001); C. A. Janeway, P. Travers, M. Walport, Immunobiology (Churchill Livingston, 2004). Preferably, the second polypeptide comprises an antibody that binds to CD20. More preferably, the second polypeptide is a monoclonal anti-CD20 antibody. Rituxan (generic name ‘Rituximab’; British trade name ‘MabThera’), the FDA approved drug for the treatment of non-Hodgkin's lymphoma, is an example of a monoclonal anti-CD20 antibody. Rituxan is a chimeric monoclonal antibody targeted against the pan-B-cell marker CD20. The terms ‘rituxan’ and ‘rituximab’, as used herein, refer to rituxan, disclosed in U.S. Pat. Nos. 5,736,137, 5,776,456, 5,843,437 and international counterparts, as well as to variants, fragments, conjugates, derivatives and modifications thereof, or other equivalent compositions with improved or optimized properties (e.g. WO 02/34790, WO 03/011878, WO 04/032828). Any suitable formulation, carrier or diluent or any other additive that may be comprised in the pharmaceutical preperation of rituxan or its equivalents is understood to be within the scope of the present invention. In certain embodiments the second polypeptide may be characterized by one or more features of the first polypeptide.

Other examples of the second polypeptide that may be used in the methods of the invention include, but are not limited to, 4D5, Mab225, C225, Daclizumab (Zenapax), Antegren, CDP 870, CMB-401, MDX-33, MDX-220, MDX-477, CEA-CIDE, AHM, Vitaxin, 3622W94, Therex, 5G1.1, IDEC-131, HU-901, Mylotarg, Zamyl (SMART M195), MDX-210, Humicade, LymphoCIDE, ABX-EGF, 17-1A, Trastuzumab (Herceptin®, rhuMAb), Epratuzumab, Cetuximab (Erbitux®), Pertuzumab (Omnitarg®, 2C4), R3, CDP860, Bevacizumab (Avastin®), tositumomab (Bexxar®), Ibritumomab tiuxetan (Zevalin®), M195, 1D10, Hu1D10 (Remitogen®, apolizumab), Danton/DN1924, an “HD” antibody such as HD4 or HD8, CAMPATH-1 and CAMPATH-1H or other variants, fragments, conjugates, derivatives and modifications thereof, or other equivalent compositions with improved or optimized properties.

The first and the second polypeptide of the present invention may also be variants of any of the above-mentioned polypeptides. A “variant”, as used herein, refers to a polypeptide with the same or similar binding specificity as a particular polypeptide, but containing sequence change(s) from the given sequence of the particular polypeptide. Such sequence changes include, for example, a change in the DNA sequence encoding the polypeptide that does not lead to amino acid change (a silent change), or a change that leads to a conservative amino acid substitution.

The modifications or variants described above for the first polypeptide are also applicable for the antibody-based antigen binding domain of the second polypeptide or other parts of the first or second polypeptide.

In certain embodiments, the first polypeptide, or the second polypeptide, or both are operably linked to a cytotoxic agent. Alternatively, the first polypeptide, or the second polypeptide, or both, are operably linked to an immunogenic agent. As a further alternative, the first polypeptide and the second polypeptide is each linked to a cytotoxic agent or an immunogenic agent, or vice versa.

In certain preferred embodiments, the antigen binding sites are cross-linked to a polymer.

The methods of the invention using both the first and the second polypeptides (the “combination treatment methods”) are suitable for treating any disorder. In certain embodiments, said disorder is a cell proliferative disorder. In certain other embodiments, said disorder is caused or contributed to by transformed cells expressing MHC class II antigens. In certain further embodiments, said disorder is caused or contributed to by unwanted activation of cells of the immune system, such as, for example, lymphoid cells expressing MHC class II. In still flier embodiments, said disorder is caused or contributed to by non-lymphoid cells that express MHC class II molecules. A disorder “caused or contributed to by” a certain factor includes a disorder that involves the factor.

The term “cell-proliferative disorder” includes both, disorders comprising benign and disorders comprising malignant cell populations that morphologically differ from the surrounding tissue. For example, tumors of the lung, breast, lymphoid, gastrointestinal, and genitourinary tract; epithelial carcinomas that include malignancies such as most colon cancers, renal-cell carcinoma, prostate cancer, non-small cell carcinoma of the lung, cancer of the small intestine, stomach cancer, kidney cancer, cervical cancer, cancer of the esophagus, and any other organ type that has a draining fluid or tissue accessible to analysis; nonmalignant cell-proliferative diseases such as colon adenomas, hyperplasia, dysplasia and other pre-malignant lesions; and transitional cell carcinoma of the bladder and head and neck cancer.

A cell proliferative disorder as described herein may be a neoplasm. Such neoplasms are either benign or malignant. The term “neoplasm” refers to a new, abnormal growth of cells or a growth of abnormal cells that reproduce faster than normal. A neoplasm creates an unstructured mass (a tumor) which can be either benign or malignant. For example; the neoplasm may be a head, neck, lung, esophageal, stomach, small bowel, colon, bladder, kidney, or cervical neoplasm. The term “benign” refers to a tumor that is noncancerous, e.g. its cells do not proliferate or invade surrounding tissues. The term “malignant” refers to a tumor that is metastastic or no longer under normal cellular growth control.

In certain further embodiments, the combination treatment methods of the invention can be used to treat disorders or conditions involving transformed cells expressing MHC class II antigens, including, for example, B cell non-Hodgkin lymphoma, B cell lymphoma, B cell acute lymphoid leukemia, Burkitt lymphoma, Hodgkin lymphoma, hairy cell leukemia, acute myeloid leukemia, T cell lymphoma, T cell non-Hodgkin lymphoma, chronic myeloid leukemia, chronic lymphoid leukemia, multiple myeloid leukemia, B cell precursor leukemia and multiple myeloma

Exemplary activated lymphoid tumor cells which can be killed include PRIESS(ECACC Accession No: 86052111), GRANTA-519 (DSMZ Accession No: ACC 342), KARPAS-422 (DSMZ Accession No: ACC 32), KARPAS-299, DOHH-2, SR-786, MHH-CALL-4, MN-60, BJAB, RAJI, L-428, HDLM-2, HD-MY-Z, KM-H2, L1236, BONNA-12, HC-1, NALM-1, L-363, EOL-1, LP-1, RPMI-8226, and MHH-PREB-1 cell lines. In certain instances, to effect cell killing, the target cells may require further activation or pre-activation, such as by incubation with Lipopolysaccharide (LPS, 10 μg/ml), Interferon-gamma (IFN-γ, Roche, 40 ng/ml) and/or phyto-hemagglutinin (PHA; 5 μg/ml) to name but a few.

Rituxan (rituximab) is also used to treat disorders involving B cells other than lymphomas, such as a variety of autoimmune diseases (reviewed e.g., in Arthritis & Rheumatism (2003), Vol. 48, p. 1484-1492). Thus, in certain embodiments, the combination treatment methods of the invention are useful to treat diseases involving unwanted activation of immune cells. For instance, the formulations can be used for the treatment of a disorder selected from rheumatoid arthritis, juvenile arthritis, multiple sclerosis, Grave's disease, insulin-dependent diabetes, narcolepsy, psoriasis, systemic lupus erythematosus, ankylosing spondylitis, transplant rejection, graft vs. host disease, Hashimoto's disease, myasthenia gravis, pemphigus vulgaris, glomerulonephritis, thyroiditis, pancreatitis, insulitis, primary biliary cirrhosis, irritable bowel disease, Sjogren syndrome, autoimmune thrombocytopenia (also known as idiopathic thrombocytopenic purpura [ITP]), systemic lupus erythematosus (SLE), autoimmune hemolytic anemia, cold agglutin disease, mixed eryoglobulinemia, neuropathies associated with autoantibodies, myasthenia gravis, Wegener's granulomatosis, and dermatomyositis.

In other embodiments, combination treatment methods, compositions or uses of the invention are useful to treat conditions involving unwanted cell proliferation, particularly the treatment of a disorder involving transformed cells expressing MHC class II antigens, such as solid tumors (see Examples 22 and 24). Solid tumors, as defined herein, refers to tumors of body tissues other than blood, bone marrow, or the lymphatic system, such as adrenocortical carcinoma, carcinoma, colorectal carcinoma, desmoid tumor, desmoplastic small round cell tumor, endocrine tumor, Ewing sarcoma family tumors, germ cell tumors, hepatoblastoma, hepatocellular carcinoma, melanoma, neurobalstoma, non-rhabdomyosarcoma soft tissue sarcoma, osteosarcoma, peripheral primitive neuroectodermal tumor, retinoblastoma, rhabdomyosarcoma and Wilms tumor.

MHC class II molecules are expressed on solid tumors, such as melanomas, in which they play a role in signaling (Brit J Cancer (1988), Vol. 58, p. 753-761; Cancer Res (1992), Vol. 52, p. 5954-5962; Cancer Biotherapy & Radiopharmaceuticals (1996), Vol. 11, p. 177-185; J Cell Sci (2003), Vol. 116, p. 2565-2575).

Another aspect of the present invention provides methods for treating a disorder comprising administering to an individual in need thereof a first polypeptide comprising a human antibody-based antigen-binding domain that binds to a human class II MHC molecule (the “single treatment method”). The single treatment methods are useful for treating a disorder involving transformed cells expressing MHC class II antigens, such as solid tumors, as defined above. In certain embodiment, the single treatment methods are useful for treating melanoma. In certain further embodiments, the melanoma is selected from: cutaneous melanoma, nodular malignant melanoma, lentiginous malignant melanoma, acral lentiginous melanoma, demoplastic malignant melanoma, giant melanocytic nevus, amelanotic malignant melanoma, acral lentiginous melanoma, mucosal malignant melanoma and ocular malignant melanoma

In other aspects of the invention, the single treatment methods or the combined treatment methods of the invention may be used in adjuvant therapy. The methods are used for the treatment of patients with cancers that are, may, or are thought to have spread outside their original sites. Adjuvant therapy may be started concurrently or after primary treatment. Primary treatment may comprise surgery, chemotherapy, radiotherapy, hormone therapy or any other therapy known to the skilled artisan, as well as any combination of these treatments. Usually adjuvant therapy is begun soon after primary therapy to delay recurrence and/or to prolong survival of the patient. Cancer cells may have metastasized to other organs of the body. Most commonly affected are the lung, liver, bone, lymph nodes, and skin.

In other aspects of the invention, the single treatment methods or the combined treatment methods of the invention may be used to treat a disorder in its terminal stage (Example 25). In preferred embodiments the disorder is selected from a disorder involving transformed cells expressing MHC class II antigens. In one embodiment the disorder is disseminated lymphoma.

Another aspect of the invention provides methods for treating a disorder comprising administering to an individual in need thereof (i) a first polypeptide comprising an antibody-based antigen-binding domain selected from: MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-647, MS-GPC-8-10-57, MS-GPC-8-27-7, MS-GPC-8-27-10, MS-GPC-8-27-41, a variant thereof or a modified version of the forgoing; and (ii) a second polypeptide comprising rituximab (RITUXAN®). In particular embodiments, the “individual in need thereof” is an animal, such as a human.

A further aspect of the invention is directed to the use of a first polypeptide comprising antibody-based antigen-binding domain selected from: MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-645, MS-GPC-8-6-13, MS-GPC-8-6-47, MS-GPC-8-10-57, MS-GPC-8-27-7, MS-GPC-8-27-10, MS-GPC-8-27-41, a variant thereof or a modified version of the forgoing, for the preparation of a pharmaceutical for the treatment of a disorder amenable to administration with said first polypeptide, wherein said first polypeptide is administered with a second polypeptide comprising rituximab (RITUXAN®).

In certain embodiments, said first and second polypeptides in the foregoing uses are administered concurrently. In certain other embodiments, said first and second polypeptides in the foregoing uses are administered sequentially.

Another aspect of the invention provides methods of killing or inhibiting the growth of a cell, comprising contacting said cell with a first polypeptide comprising a human antibody-based antigen-binding domain that binds to a human class II MHC molecule, and a second polypeptide comprising an antibody-based antigen-binding domain that binds to a cell surface receptor. The first and the second polypeptides may be contacted with said cell, such as by administration of the subject polypeptides, concurrently or sequentially, as described above. In certain embodiments, said cell is derived from or included in a tumour selected from: B cell non-Hodgkins lymphoma, B cell lymphoma, B cell acute lymphoid leukemia, Burkitt lymphoma, Hodgkins lymphoma, hairy cell leukemia, acute myeloid leukemia, T cell lymphoma, T cell non-Hodgkins lymphoma, chronic myeloid leukemia, chronic lymphoid leukemia, multiple myeloma, and multiple myeloid leukemia. In other embodiments said cell is derived from a solid tumor, such as a melanoma. Different melanoma cell lines are described in the literature. See, for example, Lawson et al., 1987, and Singh et al., 1994). Exemplary melanomas include cutaneous melanoma, nodular malignant melanoma, lentiginous malignant melanoma, acral lentiginous melanoma, demoplastic malignant melanoma, giant melanocytic nevus, amelanotic malignant melanoma, acral lentiginous melanoma, mucosal malignant melanoma and ocular malignant melanoma.

A further aspect of the present invention provides methods of killing or inhibiting the growth of a cell from a solid tumor, comprising administering to an individual in need thereof a first polypeptide comprising a human antibody-based antigen-binding domain that binds to a human class II MHC molecule. In certain embodiment, said cell is derived from Or included in a melanoma as described above.

Another aspect of the invention is directed to the use of a first polypeptide comprising an antibody-based antigen-binding domain which binds to a human class II MHC molecule for the preparation of a pharmaceutical for the treatment of a disorder amenable to administration with said first polypeptide, wherein said first polypeptide is administered with a second polypeptide comprising an antibody-based antigen-binding domain which binds to a cell surface receptor.

A further aspect of the invention is directed to the use of a second polypeptide comprising an antibody-based antigen-binding domain which binds to a cell surface receptor for the preparation of a pharmaceutical for the treatment of a disorder amenable to administration with said second polypeptide, wherein said second polypeptide is, administered with a first polypeptide comprising an antibody-based antigen-binding domain which binds to a human class II MHC molecule.

A still further aspect of the invention is directed to the use of (i) a first polypeptide comprising an antibody-based antigen-binding domain which binds to a human class II MHC molecule for the preparation of a first pharmaceutical, and (ii) a second polypeptide comprising an antibody-based antigen-binding domain which binds to a cell surface receptor for the preparation of a second pharmaceutical, for the treatment of a disorder amenable to administration with said first and/or second polypeptides.

A still further aspect of the invention is directed to the use of (i) a first polypeptide comprising an antibody-based antigen-binding domain which binds to a human class II MHC molecule, and (ii) a second polypeptide comprising an antibody-based antigen-binding domain which binds to a cell surface receptor, for the preparation of a pharmaceutical comprising both polypeptides for the treatment of a disorder amenable to administration with said first and/or second polypeptides.

In certain embodiments, said first and second polypeptides in the foregoing uses are administered concurrently. In certain other embodiments, said first and second polypeptides in the foregoing uses are administered sequentially.

Another aspect of the invention is directed to the use of a first polypeptide comprising an antibody-based antigen-binding domain which binds to a human class II MHC molecule for the preparation of a pharmaceutical for the treatment of solid tumors.

In certain embodiments the preparation of a pharmaceutical includes the manufacture of a medicament.

A further aspect of the invention provides a first polypeptide comprising an antibody-based antigen-binding domain which binds to a human class II MHC molecule for use in treating a disorder amenable to administration with said first polypeptide, wherein said first polypeptide is administered with a second polypeptide comprising an antibody-based antigen-binding domain which binds to a cell surface receptor.

Another aspect of the invention provides a second polypeptide comprising an antibody-based antigen-binding domain which binds to a cell surface receptor for use in treating a disorder amenable to administration with said second polypeptide, wherein said second polypeptide is administered with a first polypeptide comprising an antibody-based antigen-binding domain which binds to a human class II MHC molecule.

Another aspect of the invention provides two separate polypeptides, (i) a first polypeptide comprising an antibody-based antigen-binding domain which binds to a human class II MHC molecule and (ii) a second polypeptide comprising an antibody-based antigen-binding domain which binds to a cell surface receptor, for use in treating a disorder amenable to administration with said first and/or second polypeptides.

Yet another aspect of the invention provides a mixture comprising at least two polypeptides, wherein (i) a first polypeptide comprises an antibody-based antigen-binding domain which binds to a human class II MHC molecule and (ii) a second polypeptide comprises an antibody-based antigen-binding domain which binds to a cell surface receptor for use in treating a disorder amenable to administration with said first and/or second polypeptides.

Still another aspect of the invention provides a polypeptide comprising an antibody-based antigen-binding domain which binds to a human class II MHC molecule for use in the treatment of solid tumors. In certain embodiments, the solid tumor is melanoma.

Another aspect of the invention is directed to the use of a second polypeptide comprising rituximab (RITUXAN®) for the preparation of a pharmaceutical for the treatment of a disorder amenable to administration with said second polypeptide, wherein said second polypeptide is administered with a first polypeptide comprising an antibody-based antigen-binding domain selected from: MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GP C-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPC-8-27-10, MS-GPC-8-27-41, a variant thereof or a modified version of the forgoing.

Yet another aspect of the invention is directed to the use of (i) a first polypeptide comprising an antibody-based antigen-binding domain selected from: MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-645, MS-GPC-8-6-13, MS-GPC-8-647, MS-GPC-8-10-57, MS-GPC-8-27-7, MS-GPC-8-27-10, MS-GPC-8-27-41, a variant thereof or a modified version of the forgoing, for the preparation of a first pharmaceutical, and (ii) a second polypeptide comprising rituximab (RITUXAN®) for the preparation of a second pharmaceutical, for the treatment of a disorder amenable to administration with said first and/or second polypeptides.

Still another aspect of the invention is directed to the use of (i) a first polypeptide comprising an antibody-based antigen-binding domain selected from: MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-645, MS-GPC-8-6-13, MS-GPC-8-6-47, MS-GPC-8-10-57, MS-GPC-8-27-7, MS-GPC-8-27-10, MS-GPC-8-27-41, a variant thereof or a modified version of the forgoing, and (ii) a second polypeptide comprising rituximab (RITUXAN®), for the preparation of a pharmaceutical comprising both polypeptides for the treatment of a disorder amenable to administration with said first and/or second polypeptides.

Another aspect of the invention provides a first polypeptide comprising an antibody-based antigen-binding domain selected from: MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPC-8-27-10, MS-GPC-8-27-41, a variant thereof or a modified version of the forgoing, for use in treating a disorder amenable to administration with said first polypeptide, wherein said first polypeptide is administered with a second polypeptide comprising rituximab (RITUXAN®).

A further aspect of the invention provides a second polypeptide comprising rituximab (RITUXAN®) for use in treating a disorder amenable to administration with said second polypeptide, wherein said second polypeptide is administered with a first polypeptide comprising an antibody-based antigen-binding domain selected from: MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6′-27, MS-GPC-8-6-45, MS-GPC-8-6-J13, MS-GPC-8-6-47, MS-GPC-8-10-57, MS-GPC-8-27′-7, MS-GPC-8-27-10, MS-GPC-8-27-41, a variant thereof or a modified version of the forgoing.

Yet another aspect of the invention provides two separate compositions respectively including (i) a first polypeptide comprising an antibody-based antigen-binding domain selected from: MS- GPC-1, MS-GPC-8, MS-GPC-10; MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPC-8-27-10, MS-GPC-8-27-41, a variant there of or a modified version of the forgoing, and (ii) a second polypeptide comprising rituximab (RITUXAN®), for use in treating a disorder amenable to administration with said first and/or second polypeptides.

Still yet another aspect of the invention provides a mixed composition of (i) a first polypeptide comprising an antibody-based antigen-binding domain selected from: MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-645, MS-GPC-8-6-13, MS-GPC-8-647, MS-GPC-8-10-57, MS-GPC-8-27-7, MS-GPC-8-27-10, MS-GPC-8-27-41, a variant thereof or a modified version of the forgoing, and (ii) a second polypeptide comprising rituximab (RITUXAN®) for use in treating a disorder amenable to administration with said first and/or second polypeptides.

The term “a disorder amenable to administration of [an agent]” encompasses a disorder that is suitable for treatment with the agent as well as a disorder that is improved by treatment with the agent. Said term can include a disorder that a physician reasonably judges that administration of said agent is medically, experimentally or morally justified.

In other embodiments of the invention, the use or administration of the first polypeptide is to treat or ameliorate a disorder that is further amenable to administration with said second polypeptide. In particular embodiments, such a disorder would further benefit from treatment by said second polypeptide, or has been previously treated by or administered with said second polypeptide.

In other embodiments of the invention, the use or administration of the second polypeptide is to treat or ameliorate a disorder that is further amenable to administration with said first polypeptide. In particular embodiments, such a disorder would further benefit from treatment by said first polypeptide, or has been previously treated by or administered with said first polypeptide.

The term “administration with said first and/or second polypeptide”, as used herein, includes administration with either the first or the second polypeptide alone, and administration with a combination of both the first and the second polypeptides.

Another aspect of the invention provides methods of treating a disorder comprising administering to an individual in need thereof: (i) a first polypeptide comprising an antibody-based antigen-binding domain that binds to a human class II MHC molecule, and (ii) when the disorder is other than a solid tumor, said method further comprising administering to said individual a second polypeptide comprising an antibody-based antigen-binding domain that binds to a cell surface receptor. In certain further embodiments, when the disorder is a solid tumor, said method further comprises administering to said individual a second polypeptide comprising an antibody-based antigen-binding domain which binds to a cell surface receptor. In particular embodiments, the “individual in need thereof” is an animal, such as a human.

Another aspect of the invention provides compositions including an antibody-based antigen-binding domain which binds to a human class II M-C molecule, and a second polypeptide comprising an antibody-based antigen-binding domain which binds to a cell surface receptor. The compositions may further include a pharmaceutically acceptable carrier.

A further aspect of the invention provides pharmaceutical preparations comprising the compositions of the invention for treating a disorder in an animal in need thereof. Preferably, the animal is a human.

In a further embodiment, the present invention relates to the use of the composition of the present invention for preparing a pharmaceutical preparation for the treatment of animals.

Another aspect the invention provides a pharmaceutical package for treating an individual suffering from a disorder, wherein said package includes comprising a first polypeptide comprising an antibody-based antigen-binding domain which binds to a human class II MHC molecule, and a second polypeptide comprising an antibody-based antigen-binding domain which binds to a cell surface receptor. In certain embodiments, the first and the second polypeptides are formulated separately and in individual dosage amounts. In certain other embodiments, the first and the second polypeptides are formulated together and in individual dosage amounts. In certain other embodiments, the first and the second polypeptides are formulated separately and in individual dosage amounts. In certain still further embodiments, the pharmaceutical package comprises instructions to treat the disorder.

In yet another aspect the invention provides a pharmaceutical package for treating an individual suffering from a solid tumor disorder, wherein said package includes comprising a first polypeptide comprising an antibody-based antigen-binding domain which binds to a human class II MHC molecule. In certain still further embodiments, the pharmaceutical package comprises instructions to treat the disorder.

The invention further relates to a diagnostic composition containing at least one polypeptide and/or nucleic acid comprising/encoding an antibody-based antigen-binding domain which binds to a human class II MHC molecule, optionally together with further reagents, such as a second polypeptide comprising an antibody-based antigen-binding domain which binds to a cell surface receptor, or a nucleic acid encoding the same, and/or buffers, for performing the diagnosis.

In a preferred embodiment the diagnostic composition contains the polypeptide comprising an antibody-based antigen-binding domain which binds to a human class II MHC molecule according to the invention cross-linked by at least one moiety. Such moieties can be for example antibodies recognizing an epitope present on the polypeptide such as the FLAG peptide epitope (Hopp et al., 1988; Knappik and Plückthun, 1994) or bifunctional chemical compounds reacting with a nucleophilic amino acid side chain as present in cysteine or lysine (King et al., 1994). Methods for cross-linking polypeptides are well known to the practitioner of ordinary skill in the art.

A diagnostic composition containing at least one nucleic acid encoding a subject polypeptide and/or variant thereof according to the invention is also contemplated.

In certain embodiments of any of the aspects of the invention described herein, including the methods, uses, compositions, compositions for the uses described and pharmaceutical packs/compositions, the first polypeptide can comprise a human antibody-based antigen-binding domain. In alternate embodiments of such aspects, the first polypeptide can comprise an antibody-based antigen-binding domain of human composition. In further alternative embodiments of such aspects, the first polypeptide can comprise an antibody-based antigen-binding domain that is not a humanized or not a chimeric antigen-binding domain or antibody. In yet further alternative embodiments of such aspects, the first polypeptide can comprise Danton/DN1924/DN1921 (Dendreon) or an “HD” antibody such as HD4 or HD8 (Kirin).

Pharmaceutical Preparations and Methods of Administration

According to the methods of the invention, the subject polypeptide(s) may be administered in a pharmaceutically acceptable composition or compositions. In general, pharmaceutically-acceptable carriers for monoclonal antibodies, antibody fragments, and peptides are well-known to those of ordinary skill in the art. As used herein, the term “pharmaceutically acceptable carrier” includes any and all solvents, dispersion media, coatings, antibacterial and antifungal agents, isotonic and absorption delaying agents and the like. In preferred embodiments, the subject carrier medium which does not interfere with the effectiveness of the biological activity of the active ingredients and which is not excessively toxic to the hosts of the concentrations of which it is administered. The administration(s) may take place by any suitable technique, including subcutaneous and parenteral administration, preferably parenteral. Examples of parenteral administration include intravenous, intraarterial, intramuscular, and intraperitoneal, with intravenous being preferred.

The pharmaceutical forms suitable for injectable use include sterile aqueous solutions or dispersions and sterile powders for the extemporaneous preparation of sterile injectable solutions or dispersions. In such cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds, e.g., the subject polypeptides, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Upon formulation, solutions can be administered in a manner compatible with the dosage formulation and in such amount as is therapeutically effective. The formulations are easily administered in a variety of dosage forms such as injectable solutions, drug release capsules and the like.

As used herein the term “pharmaceutical package” or “pharmaceutical pack” refer to any packaging system for storing and dispensing individual doses of medication. Preferably the pharmaceutical package contains sufficient daily dosage units appropriate to the treatment period or in amounts which facilitate the patient's compliance with the regimen. In certain embodiments, the pharmaceutical package comprises one or more vessels that include the active ingredient, e.g. a subject polypeptide. Such vessel can be a container such as a bottle, vial, syringe or capsule, or may be a unit dosage form such as a pill. The active ingredient may be provided in the vessel in a pharmaceutically acceptable form or may be provided e.g. as a lyophilized powder. In further embodiments, the pharmaceutical, package may can further include a solvent to prepare the active ingredient for administration. In certain embodiments, the active ingredient may be already provided in a delivery device, such as a syringe, or a suitable delivery device may be included in the package. The pharmaceutical package may comprise pills, liquids, gels, tablets, dragees or the pharmaceutical preparation in any other suitable form. The package may contain any number of daily pharmaceutical dosage units. The package may be of any shape, and the unit dosage forms may be arranged in any pattern, such as circular, triangular; trapezoid, hexagonal or other patterns. One or more of the doses or subunits may be indicated, for example to aid the doctor, pharmacist or patient, by identifying such dose or subunits, such as by employing color-coding, labels, printing, embossing, scorings or patterns. The pharmaceutical package may also comprise instructions for the patient, the doctor, the pharmacist or any other related person.

Some embodiments comprise the administration of two polypeptides. Such administration may occur concurrently or sequentially. The polypeptides may be formulated together such that one administration delivers both components. Alternatively the polypeptides may be formulated separately. The pharmaceutical package may comprise the first and the second polypeptide in a single formulation, i.e. they are formulated together, or the first and the second polypeptides in individual formulations, i.e. they are formulated separately. Each formulation may comprise the first polypeptide and/or the second polypeptide in individual dosage amounts. Administration of each polypeptide of the combination results in a concentration of the polypeptide that, combined with the other polypeptide, results in a therapeutically effective amount of the combination.

Still another aspect of the present invention provides a host cell harboring at least one subject nucleic acids or the subject vector. Another aspect of the present invention provides a method for the production of a multivalent composition that causes or leads to killing of cells in a manner where neither cytotoxic entities nor immunological mechanisms are needed to cause or lead to said killing comprising culturing the host cells under conditions wherein the nucleic acid is expressed either as a polypeptide comprising a plurality of antigen binding domains or as a polypeptide comprising at least one antigen binding domains which is subsequently treated to form a multivalent composition.

Another aspect of the present invention provides forms of the subject polypeptide or nucleic acid compositions, formulated in a pharmaceutically acceptable carrier and/or diluent. The present invention specifically contemplates the use of such compositions for preparing a pharmaceutical preparation for the treatment of animals, especially humans.

Definitions

As used herein, the term “peptide” relates to molecules consisting of one or more chains of multiple, i.e. two or more, amino acids linked via peptide bonds.

The term “protein” refers to peptides where at least part of the peptide has or is able to acquire a defined three-dimensional arrangement by forming secondary, tertiary, or quaternary structures within and/or between its peptide chain(s). This definition comprises proteins such as naturally occurring or at least partially artificial proteins, as well as fragments or domains of whole proteins, as long as these fragments or domains are able to acquire a defined three-dimensional arrangement as described above.

The term “polypeptide” is used interchangeably to refer to peptides and/or proteins. Moreover, the terms “polypeptide” and “protein”, as the context will admit, include multi-chain protein complexes, such as immunoglobulin polypeptides having separate heavy and light chains.

In this context, “polypeptide comprising at least one antibody-based antigen-binding domain” refers to an immunoglobulin (or antibody) or to a fragment thereof. The term “fragment”, with respect to antibody domains and the like, refers to a fragment of an immunoglobulin which retains the antigen-binding moiety of an immunoglobulin. Functional immunoglobulin fragments according to the present invention may be Fv (Skerra and Pluckthun, 1988), scFv (Bird et al., 1988; Huston et al., 1988), disulfide-linked Fv (Glockshuber et al., 1992; Brinkmann et al., 1993), Fab, F(ab′)2 fragments or other fragments well-known to the practitioner skilled in the art, which comprise the variable domains of an immunoglobulin or functional immunoglobulin fragment.

Examples of polypeptides consisting of one chain are single-chain Fv antibody fragments, and examples for polypeptides consisting of multiple chains are Fab antibody fragments.

The term “antibody” as used herein, unless indicated otherwise, is used broadly to refer to both antibody molecules and a variety of antibody derived molecules. Such antibody derived molecules comprise at least one variable region (either a heavy chain of light chain variable region) and include such fragments as described above, as well as individual antibody light chains, individual antibody heavy chains, chimeric fusions between antibody chains and other molecules, and the like.

The “antigen-binding site” of an immunoglobulin molecule refers to that portion of the molecule that is necessary for binding specifically to an antigen. An antigen binding site preferably binds to an antigen with a K_{d} of 1 μM or less, and more preferably less than 100 nM, 10 nM or even 1 nM in certain instances. Binding specifically to an antigen is intended to include binding to the antigen which significantly higher affinity than binding to any other antigen.

The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

As used herein, the phrase “conservative amino acid substitution” refers to grouping of amino acids on the basis of certain common properties. A functional way to define common properties between individual amino acids is to analyze the normalized frequencies of amino acid changes between corresponding proteins of homologous organisms (Schulz, G. E. and R. H. Schirmer, Principles of Protein Structure, Springer-Verlag). According to such analyses, groups of amino acids may be defined where amino acids within a group exchange preferentially with each other, and therefore resemble each other most in their impact on the overall protein structure (Schulz, G. E. and R. H. Schirmer, Principles of Protein Structure, Springer-Verlag). Examples of amino acid groups defined in this manner include:

(i) a charged group, consisting of Glu and Asp, Lys, Arg and His,

(ii) a positively-charged group, consisting of Lys, Arg and His,

(iii) a negatively-charged group, consisting of Glu and Asp,

(iv) an aromatic group, consisting of Phe, Tyr and Trp,

(v) a nitrogen ring group, consisting of His and Trp,

(vi) a large aliphatic nonpolar group, consisting of Val, Leu and Be,

(vii) a slightly-polar group, consisting of Met and Cys,

(viii) a small-residue group, consisting of Ser, Thr, Asp, Asn, Gly, Ala, Glu, Gln and Pro,

(ix) an aliphatic group consisting of Val, Leu, fle, Met and Cys, and

(x) a small hydroxyl group consisting of Ser and Thr.

For the purposes of this application, “valent” refers to the number of antigen binding sites the subject polypeptide possess. Thus; a bivalent polypeptide refers to a polypeptide with two binding sites. The term “multivalent polypeptide” encompasses bivalent, trivalent, tetravalent, etc. forms of the polypeptide.

As used herein, a “multivalent composition” or “multivalent polypeptide” means a composition or polypeptide including at least two of said antigen-binding domains. Preferably, said at least two antigen-binding domains are in close proximity so as to mimic the structural arrangement relative to each other of binding sites comprised in a full immunoglobulin molecule. Examples for multivalent compositions are full immunoglobulin-molecules (e.g. IgG, IgA or IgM molecules) or multivalent fragments thereof (e.g. F(ab′)₂). Additionally, multivalent compositions of higher valencies may be formed from two or more multivalent compositions (e.g. two or more full immunoglobulin molecules), e.g. by cross-linking. Multivalent compositions, however, may be formed as well from two or more monovalent immunoglobulin fragments, e.g. by self-association as in mini-antibodies, or by cross-linking.

Accordingly, an “antibody-based antigen-binding domain” refers to polypeptide or polypeptides which form an antigen-binding site retaining at least some of the structural features of an antibody, such as at least one CDR sequence. In certain preferred embodiments, antibody-based antigen-binding domain includes sufficient structure to be considered a variable domain, such as three CDR regions and interspersed framework regions. Antibody-based antigen-binding domain can be formed single polypeptide chains corresponding to VH or VL sequences, or by intermolecular or intramolecular association of VH and VL sequences.

The term “recombinant antibody library” describes a variegated library of antigen binding domains. For instance, the term includes a collection of display packages, e.g., biological particles, which each have (a) genetic information for expressing at least one antigen binding domain on the surface of the particle, and (b) genetic information for providing the particle with the ability to replicate. For instance, the package can display a fusion protein including an antigen binding domain. The antigen binding domain portion of the fusion protein is presented by the display package in a context which permits the antigen binding domain to bind to a target epitope that is contacted with the display package. The display package will generally be derived from a system that allows the sampling of very large variegated antibody libraries. The display package can be, for example, derived from vegetative bacterial cells, bacterial spores, and bacterial viruses.

In an exemplary embodiment of the present invention, the display package is a phage particle which comprises a peptide fusion coat protein that includes the amino acid sequence of a test antigen binding domains. Thus, a library of replicable phage vectors, especially phagemids (as defined herein), encoding a library of peptide fusion coat proteins is generated and used to transform suitable host cells. Phage particles formed from the chimeric protein can be separated by affinity selection based on the ability of the antigen binding site associated with a particular phage particle to specifically bind a target eptipope. In a preferred embodiment, each individual phage particle of the library includes a copy of the corresponding phagemid encoding the peptide fusion coat protein displayed on the surface of that package. Exemplary phage for generating the present variegated peptide libraries include M13, fl, fd, Ifl, Ike, Xf, Pf1, Pf3, λ, T4, T7, P2, P4, φX-174, MS2 and f2.

The term “generating a library of variants of at least one of the CDR1, CDR2 and CDR3” refers to a process of generating a library of variant antigen binding sites in which the members of the library differ by one or more changes in CDR sequences, e.g., not FR sequences. Such libraries can be generated by random or semi-random mutagenesis of one or more CDR sequences from a selected antigen binding site.

As used herein, a “antibody-based antigen-binding domain of human composition” preferably means a polypeptide comprising at least an antibody VH domain and an antibody VL domain, wherein a homology search in a database of protein sequences comprising immunoglobulin sequences results for both the VH and the VL domain in an immunoglobulin domain of human origin as hit with the highest degree of sequence identity. Such a homology search may be a BLAST search, e.g. by accessing sequence databases available through the National Center for Biological Information and performing a “BasicBLAST” search using the “blastp” routine. See also Altschul et al. (1990) J Mol Biol 215:403-410. Preferably, such a composition does not result in an adverse immune response thereto when administered to a human recipient. In certain preferred embodiments, the subject human antigen-binding domains include the framework regions of native human immunoglobulins, as may be cloned from activated human B cells, though not necessarily all of the CDRs of a native human antibody.

As used herein the term “human antibody-based antigen-binding domain” refers to a polypeptide comprising at least an antibody VH domain and an antibody VL domain, wherein at least the framework regions of the VH domain and the VL domain, or the sequences encoding such domains, are of direct or indirect human origin. Preferably, the framework regions of the VH or VL domain show less than 15, more preferably less than 10, and most preferably less than 8, changes of amino acid residues when compared to the corresponding human germline sequence exhibiting the closest sequence homology. For example, such polypeptide may be of a natural origin and isolated from human sera, or may be isolated from a natural antibody repertoire, either by monoclonal hybridoma technology (G. Subramanian, Antibodies, Kluwer Academic/Plenum Publishers, 2004; Margaret E. Schelling, Monoclonal Antibody Protocols, Humana Press, 2002; David J. King, Application and Engineering of Monoclonal Antibodies, CRC Press 1998), or from screening of the cloned gene-library (WO 90/05144). Depending on the way of cloning and constructing such repertoire, the 3′ and/or 5′ amino acid sequences and/or one of more CDR sequences may be of at least partially synthetic origin. Alternatively, such polypeptide may be of a synthetic origin, preferably based on or homologous to the framework amino-acid or nucleic acid sequences of human immunoglobulin genes. By ways of a non-limiting example, the polypeptide comprising an antibody VH domain and an antibody VL domain may be generated by employing the methods described in Knappik et al. (2000). The Human Combinatorial Antibody Libraries (HuCAL) is a library of human-derived antibody genes by the use of synthetic consensus sequences which cover the structural repertoire of antibodies encoded in the human genome. See EP1143006A1, EP0859841B and Knappik et al. (2000), the entirety content of both of which are incorporated herein. In HuCAL, one or more of the CDR regions of VH and VL domains are diversified according to the natural distribution of amino acid residues in such CDR(s) of human antibodies. Examples of human antibody-based antigen-binding domains that bind a MHC II molecule are described in WO 01/87337. The polypeptide comprising an antibody VH domain and an antibody VL domain may also be generated using other techniques known in the art for production such polypeptides, including, for example, phage display library (U.S. Pat. No. 5,667,988) and yeast display library (Feldhaus et al., Nat. Biotechnol. 2003 February; 21(2):163-70; 2003). Such human antibody-based antigen binding domains, once isolated or identified may be further changed to form variants or modifications to maintain, or improve the properties of the parental antigen-binding domain.

As used herein, the term “mini-antibody fragment” means a multivalent antibody fragment comprising at least two antigen-binding domains multimerized by self-associating domains fused to each of said domains (Pack, 1994), e.g. dimers comprising two scFv fragments, each fused to a self-associating dimerization domain. Dimerization domains, which are particularly preferred, include those derived from a leucine zipper (Pack and Pluckthun, 1992) or helix-turn-helix motif (Pack et al., 1993).

As used herein, “activated cells” means cells of a certain population of interest, which are not resting. Activation might be caused by mitogens (e.g., lipopoysaccharide, phytohemagglutinine) or cytokines (e.g., interferon gamma). Preferably, said activation occurs during tumor transformation (e.g., by Epstein-Barr virus, or “spontaneously”). Preferably, activated cells are characterized by the features of MHC class II molecules expressed on the cell surface and one or more additional features including increased cell size, cell division, DNA replication, expression of CD45 or CD11 and production/secretion of immunoglobulin.

As used herein, “non-activated cells” means cells of a population of interest, which are resting and non-dividing. Said non-activated cells may include resting B cells as purified from healthy human blood. Such cells can, preferably, be characterized by lack or reduced level of MHC class II molecules expressed on the cell surface and lack or reduced level of one or more additional features including increased cell size, cell division, DNA replication, expression of CD45 or CD11 and production/secretion of immunoglobulin.

As used herein, the term “EC₅₀” means the concentration of multivalent forms of the subject compositions which produces 50% of its maximum response or effect, such as cell killing.

“At least 5-fold lower EC₅₀” means that the concentration of a multivalent composition comprising at least one polypeptide of the present invention that is required to kill 50% of activated cells is at least five times less than the concentration of the multivalent composition required to kill non-activated cells. Preferably, the concentration required to kill 50% of non-activated cells cannot be achieved with therapeutically appropriate concentrations of the multivalent composition. Most preferably, the EC₅₀ value is determined in the test described below in the appended examples.

The term “immunosuppress” refers to the prevention or diminution of the image response, as by irradiation or by administration of antimetabolites, antilymphocyte serum, or specific antibody.

The term “immune response” refers to any response of the immune system, or a cell forming part of the immune system (lymphocytes, granulocytes, macrophages, etc), to an antigenic stimulus, including, without limitation, antibody production, cell-mediated immunity, and immunological tolerance.

As used herein, the term “IC₅₀” with respect to immunosuppression, refers to the concentration of the subject compositions which produces 50% of its maximum response or effect, such as inhibition of an immune response, such as may be manifest by inhibition of IL2 secretion, down-regulation of IL2 expression, or reduced rate of cell proliferation.

The phrase “cytotoxic entities”, with reference to a manner of cell killing, refers to mechanisms which are complement-dependent or make use of toxicological or radiological “warheads”. Likewise, the phrase “immuological mechanism”, with reference to a manner of cell killing, refers to macrophage-dependent and/or neutrophil-dependent killing of cells.

Killing of cells in a manner where “neither cytotoxic entities nor immunological mechanisms” are needed refers to a mechanism which is mediated through an innate pre-programmed mechanism of the activated cell. For example neither “killer cells” nor complement are required for killing of lymphoid tumor cells when contacted by the antibody 1D09C3, as described in the examples herein.

The term “innate pre-programmed process” refers to a process that, once it is started, follows an autonomous cascade of mechanisms within a cell, which does not require any further auxillary support from the environment of said cell in order to complete the process. Such processes that cause cell death can include mechanisms commonly understood in the art as “apoptosis”, and can also include cell death induced by a multivalent polypeptide comprising at least two human antibody-based antigen-binding domains that bind to a human class II MHC molecule, such as 1D09C3, where such cell death is independent of caspase inhibition by zDEVD-fin or zVAD-fmk.

“Lymphoid cells” when used in reference to a cell line or a cell, means that the cell line or cell is derived from the lymphoid lineage. “Lymphoid cells” include cells of the B and the T lymphocyte lineages, and of the macrophage lineage.

Cells, which are “non lymphoid cells and express MHC class II”, are cells other than lymphoid cells that express MHC class II molecules, e.g. during a pathological inflammatory response. For example, said cells may include synovial cells, endothelial cells, thyroid stromal cells, glial cells and non-lymphoid tumor cells, such cells derived from or included in solid tumors, e.g. a melanoma Said cells may also comprise genetically altered cells capable of expressing MHC class II molecules.

The terms “apoptosis” and “apoptotic activity” refer to the form of cell death in mammals that is accompanied by one or more characteristic morphological and biochemical features, including nuclear and condensation of cytoplasm, chromatin aggregation, loss of plasma membrane microvilli, partition of cytoplasm and nucleus into membrane bound vesicles (apoptotic bodies) which contain ribosomes, morphologically intact mitochondria and nuclear material, degradation of chromosomal DNA or loss of mitochondrial function. Apoptosis follows a very stringent time course and is executed by caspases, a specific group of proteases. Apoptotic activity can be determined and measured, for instance, by cell viability assays, Annexin V staining or caspase inhibition assays. Apoptosis can be induced using a cross-linking antibody such as anti-CD95 as described in Example H.

As used herein, the term “first domain of the α-chain of HLA-DR” means the N-terminal domain of the alpha-chain of the MHC class II DR molecule.

As used herein, the term “first domain of the 5-chain of HLA-DR” means the N-terminal domain of the beta-chain of the MHC class II DR molecule.

As used herein, the term “HuCAL” refers to a fully synthetic human combinatorial antibody library as described in Knappik et al. (2000).

The term “variable region” as used herein in reference to immunoglobulin molecules has the ordinary meaning given to the term by the person of ordinary skill in the act of immunology. Both antibody heavy chains and antibody light chains may be divided into a “variable region” and a “constant region”. The point of division between a variable region and a heavy region may readily be determined by the person of ordinary skill in the art by reference to standard texts describing antibody structure, e.g., Kabat et al “Sequences of Proteins of Immunological Interest: 5th Edition” U.S. Department of Health and Human Services, U.S. Government Printing Office (1991).

As used herein, the term “CDR3” refers to the third complementarity-determining region of the VH and VL domains of antibodies or fragments thereof, wherein the VH CDR3 covers positions 95 to 102 (Kabat numbering; possible insertions after positions 100 listed as 100a to 100z), and VL CDR3 positions 89 to 96 (possible insertions in Vλ after position 95 listed as 95a to 95c) (see Knappik et al., 2000).

As used herein, the term “hybridizes under stringent conditions” is intended to describe conditions for hybridization and washing under which nucleotide sequences at least 60% homologous to each other typically remain hybridized to each other. Preferably, the conditions are such that sequences at least 65%, more preferably at least 70%, and even more preferably at least 75% homologous to each other typically remain hybridized to each other. Such stringent conditions are known to those skilled in the art and can be found in Current Protocols in Molecular Biology, John Wiley & Sons, New York. (1989), 6.3.1-6.3.6. A preferred, non-limiting example of stringent hybridization conditions is hybridization in 6× sodium chloride/sodium citrate (SSC) at about 45° C., followed by one or more washes in 0.2×SSC, 0.1% SDS at 50°-65° C.

The term “immunosuppression” as used herein refers to suppression of immune response resulting from T-cell activation, particularly antigen-mediated T-cell activation; T-cell activation by antigen can be measured by a variety of art-recognized methods. For example, IL-2 secretion by activated T-cells can be used to measure antigen-stimulated T-cell activation. Alternatively, T-cell proliferation as measured by a number of art-recognized methods (such as ³H-labeled dNTP incorporation into replicating DNA) can be used to monitor antigen-induced T-cell activation. Immunesuppression of T-cell activation by mAb's or fragments thereof refers to suppression of immune response as measured by any one of the proper methods (such as the ones mentioned above) by at least about 50%, or 60%, more preferably at least about 70% or 80%, most preferably at least about 85% or even 90%, 95%, 99%.

A “protein coding sequence” or a sequence which “encodes” a particular polypeptide or peptide, is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from procaryotic or eukaryotic mRNA, genomic DNA sequences from procaryotic or eukaryotic DNA, and even synthetic DNA sequences. A transcription termination sequence will usually be located 3′ to the coding sequence.

Likewise, “encodes”, unless evident from its context, will be meant to include DNA sequences which encode a polypeptide, as the term is typically used, as well as DNA sequences which are transcribed into inhibitory antisense molecules.

As used herein, the term “transfection” means the introduction of a heterologous nucleic acid, e.g., an expression vector, into a recipient cell by nucleic acid-mediated gene transfer. “Transient transfection” refers to cases where exogenous DNA does not integrate into the genome of a transfected cell, e.g., where episomal DNA is transcribed into mRNA and translated into protein. A cell has been “stably transfected” with a nucleic acid construct when the nucleic acid construct is capable of being inherited by daughter cells.

“Expression vector” refers to a replicable DNA construct used to express DNA which encodes the desired protein and which includes a transcriptional unit comprising an assembly of (1) agent(s) having a regulatory role in gene expression, for example, promoters, operators, or enhancers, operatively linked to (2) a DNA sequence encoding a desired protein (such as a polypeptide of the present invention) which is transcribed into mRNA and translated into protein, and (3) appropriate transcription and translation initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

In the expression vectors, regulatory elements controlling transcription or translation can be generally derived from mammalian, microbial, viral or insect genes The ability to replicate in a host, usually conferred by an origin of replication, and a selection gene to facilitate recognition of transformants may additionally be incorporated. Vectors derived from viruses, such as retroviruses, adenoviruses, and the like, may be employed.

“Transcriptional regulatory sequence” is a generic term used throughout the specification to refer to DNA sequences, such as initiation signals, enhancers, and promoters and the like which induce or control transcription of protein coding sequences with which they are operably linked. It will be understood that a recombinant gene can be under the control of transcriptional regulatory sequences which are the same or which are different from those sequences which control transcription of the naturally-occurring form of the gene, if any.

“Operably linked” when describing the relationship between two DNA regions simply means that they are functionally related to each other. For example, a promoter or other transcriptional regulatory sequence is operably linked to a coding sequence if it controls the transcription of the coding sequence.

As used herein, the term “fusion protein” is art recognized and refer to a chimeric protein which is at least initially expressed as single chain protein comprised of amino acid sequences derived from two or more different proteins, e.g., the fusion protein is a gene product of a fusion gene.

As used herein, “proliferating” and “proliferation” refer to cells undergoing mitosis.

The “growth rate” of a cell refers to the rate of proliferation of the cell and the state of differentiation of the cell.

The term “cell-proliferative disorder” denotes malignant as well as nonmalignant populations of transformed cells which morphologically often appear to differ from the surrounding tissue.

As used herein, “transformed cells” refers to cells which have spontaneously converted to a state of unrestrained growth, i.e., they have acquired the ability to grow through an indefinite number of divisions in culture. Transformed cells may be characterized by such terms as neoplastic, anaplastic and/or hyperplastic, with respect to their loss of growth control.

As used herein, “immortalized cells” refers to cells which have been altered via chemical and/or recombinant means such that the cells have the ability to grow through an indefinite number of divisions in culture.

As used herein the term “animal” refers to mammals, preferably mammals such as humans. Likewise, a “patient” or “subject” to be treated by the method of the invention can mean either a human or non-human animal.

As used herein, the term “instructions” means a product label and/or documents describing relevant materials or methodologies pertaining to assembly, preparation or use of a kit or packaged pharmaceutical. These materials may include any combination of the following: background information, steps or procedures to follow, list of components, proposed dosages, warnings regarding possible side effects, instructions for administering the drug, technical support, and any other related documents.

As used herein, the term “treating” refers to preventing a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and relieving one or more symptoms the disease, disorder or condition, i.e., causing regression of the disease, disorder and/or condition.

As used herein, the term “prophylactic or therapeutic” treatment refers to administration to the host of the medical condition. If it is administered prior to exposure to the condition, the treatment is prophylactic (i.e., it protects the host against tumor formation), whereas if administered after initiation of the disease, the treatment is therapeutic (i.e., it combats the existing tumor).

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

It is contemplated that all embodiments described above are applicable to all different aspects of the invention. It is also contemplated that any of the above embodiments can be freely combined with one or more other such embodiments whenever appropriate. In particular, various embodiments of the first and the second polypeptides, various embodiments of the disorders suitable for treatment with the methods of the present invention, and various embodiments of treatment methods may be freely combined with one another.

Specific embodiments of the invention are described in more detail below. However, these are illustrative embodiments, and should not be construed as limiting in any respect.

### BRIEF DESCRIPTION OF THE DRAWINGS

FIG. 1 a) Specificity of the anti-HLA-DR antibody fragments: Binding of MS-GPC-8-27-7, MS-GPC-8-27-10, MS-GPC-8-6-13, MS-GPC-8-27-41, MS-GPC-8-647, MS-GPC-8-10-57, MS-GPC-8-6-27, MS-GPC-8 and MS-GPC-8-6 to HLA-DR protein, negative control proteins (BSA, testosterone-BSA, lysozyme and human apotransferrin), and an empty microtiter plate well (plastic). Specificity was assessed using standard ELISA procedures. b) Specificity of the anti-HLA-DR antibody fragments MS-GPC-1, 6, 8 & 10 isolated from the HuCAL library to HLA-DR protein, a mouse-human chimeric HLA protein and negative control proteins (lysozyme, transferrin, BSA and human β-globulin). Specificity was assessed using standard ELISA procedures. A non-related antibody fragment (irr. scFv) was used as control. c) Specificity of anto-HLA-DR antibody fragments (scFv) and some of the corresponding mAb's in IgG format against a panel of human or mousr HLA-DR antigens and unrelated control antigens.

FIG. 2 Reactivity of the anti-HLA-DR antibody fragments (MS-GPC-1, 6, 8 and 10, etc.) and IgG forms of MS-GPC-8, MS-GPC-8-10-57, MS-GPC-8-27-41 & MS-GPC-8-6-17 to various cell lines expressing MHC class II molecules. “+” represents strong reactivity as detected using standard immunofluorescence procedure. “+/−” represents weak reactivity and “−” represents no detected reactivity between an anti-HLA-DR antibody fragment or IgG and a particular cell line. Percentage of cells killed by each anti-HLA-DR antibody fragments (scFv) and some of the corresponding mAb's in IgG format are also presented. Values greater than 50% are in bold.

FIG. 3 Viability of tumor cells in the presence of monovalent and cross-linked anti-HLA-DR antibody fragments as assessed by trypan blue staining. Viability of GRANTA-519 cells was assessed after 4 h incubation with anti-HLA-DR antibody fragments (MS-GPC-1, 6, 8 and 10) with and without anti-FLAG M2 mAb as cross-linking agent.

FIG. 4 Scatter plots and fitted logistic curves of data from Table 5 showing improved killing efficiency of 50 nM solutions of the IgG form of the human antibody fragments of the invention treated compared to treatment with 200 nM solutions of murine antibodies. Open circles represent data for cell lines treated with the murine antibodies L243 and 8D1 and closed circles for human antibodies MS-GPC-8, MS-GPC-8-27-41, MS-GPC-8-10-57 and MS-GPC-8-6-13. Fitted logistic curves for human (solid) and mouse (dashed) mAb cell killing data show the overall superiority of the treatment with human in mAbs at 50 nM compared to the mouse mAbs despite treatment at a final concentration of 200 nM.

FIG. 5 Killing of activated versus non-activated cells. The lymphoma line MHH-PREB-1 cells are activated with Lipopolysaccharide, Interferon-gamma and phyto-hemagglutin, and subsequently incubated for 4 hr with 0.07 to 3300 nM of the IgG forms of the anti-HLA-DR antibody fragments MS-GPC-8-10-57 and MS-GPC-8-27-41. No loss of viability in the control non-activated MHH-PREB-1 cells is seen. Viable cell recovery is expressed as % of untreated controls.

FIG. 6 a) Killing efficiency of control (no antibody, unreactive murine IgG; light grey), and human (MS-GPC-8, MS-GPC-8-10-57 & MS-GPC-8-27-41; dark grey) IgG forms of anti-HLA-DR antibody fragments against CLL cells isolated from patients. Left panel, box-plot display of viability data from 10 patient resting cell cultures against antibodies after incubation for four (h4) and twenty four hours (h24). Right panel box-plot display of viability data from 6 patient activated cell cultures against antibodies after incubation for four (h4) and twenty four hours (h24). b & c) Killing efficiency of human (B8, 1C7277 & 1D09C3) and control murine (L243 & 10F12) anti-DR mAbs against CLL cells isolated from patients. Average % viable cell recovery determined by fluorescence microscopy±S.D. of CLL cells from 10 patients is shown after 4 h or 24 h incubation with 100 nM of mAbs, compared to untreated cells. All cell samples showed strong DR expression (mean fluorescence intensity 123-865 by FACS analysis using FITC-L243). In 6c, data from activated vs. resting cells are compared.

FIG. 7 Concentration dependent cell viability for certain anti-HLA-DR antibody fragments of the invention. Vertical lines indicate the EC₅₀ value estimated by logistic non-linear regression on replica data obtained for each of the antibody fragments. a) Killing curves of cross-linked bivalent anti-HLA-DR antibody F(ab) fragment dimers MS-GPC-10 (circles and solid line), MS-GPC-8 (triangles and dashed line) and MS-GPC-1 (crosses and dotted line). b) Killing curves of cross-linked bivalent anti-HLA-DR antibody (Fab) fragment dimers MS-GPC-8-17 (circles and solid line), and murine IgGs 8D1 (triangles and dashed line) and L243 (crosses and dotted line). c) Killing curves of cross-linked bivalent anti-HLA-DR antibody (Fab) fragment dimers GPC-8-6-2 (crostriangles and dashed line), and murine IgGs 8D1 (circles and solid line) and L243 (crosses and dotted line). d) Killing curves of IgG forms of human anti-HLA-DR antibody fragments MS-GPC-8-10-57 (crosses and dotted line), MS-GPC-8-27-41 (exes and dash-dot line), and murine IgGs 8D1 (circles and solid line) and L243 (triangles and dashed line). All concentrations are given in nM of the bivalent agent (IgG or cross-linked (Fab) dimer).

FIG. 8 Mechanism and selectivity of anti-DR induced cell death. a) Comparison of death induced in PRIESS cells by the Fab fragment of human anti-DR mAb B8 crosslinked with anti-FLAG, and anti-CD95 mAb, respectively. Incubation of PRIESS cells with the anti-HLA-DR antibody fragment MS-GPC-8, cross-linked using the anti-FLAG M2 mAb, shows more rapid killing than a culture of PRIESS cells induced into apoptosis using anti-CD95 mAb. An Annexin V/PI staining technique identifies necrotic cells by Annexin V positive and PI positive staining. b) Comparison of apoptosis induced in PRIESS cells after anti-DR and anti-CD95 mAb treatment. Incubation of PRIESS cells with the anti-HLA-DR antibody fragment MS-GPC-8, cross-linked using the anti-FLAG M2 mAb, shows little evidence of an apoptotic mechanism compared to an apoptotic culture of PRIESS cells induced using anti-CD95 mAb. An Annexin V/PI staining technique identifies apoptotic cells by Annexin V positive and PI negative staining. c) Activated but not resting normal human B cells are killed by anti-DR mAb treatment. B cells isolated from PBL by magnetic sorting (B Cell Isolation Kit, Miltenyi Biotec, Bergisch-Gladbach, Germany) were treated with 50 nM of different mAbs (unactivated), or stimulated with pokeweed mitogen (Gibco BRL) for 3 days (activated) and treated with mAbs subsequently.

FIG. 9 a) Immunosuppressive properties of the IgG forms of the anti-HLA-DR antibody fragments MS-GPC-8-10-57, MS-GPC-8-27-41 & MS-GPC-8-6-13 using an assay to determine inhibition of IL-2 secretion from T-hybridoma cells. b) Immunosuppressive properties of the monovalent Fab forms of the anti-HLA-DR antibody fragments MS-GPC-8-27-41 & MS-GPC-8-6-19 using an assay to determine inhibition of IL-2 secretion from T-hybridoma cells. c) Secretion of IL-2 by T-cell hybridoma Hyb1 is inhibited by human and mouse HLA-DR mAb's. d) T-cell proliferation is inhibited by mouse and human HLA-DR mAb's. e) T-cell proliferation stimulated by specific antigen hen egg lysozyme (HEL) is inhibited by mouse and human HLA-DR mAb's ex vivo. f) T-cell proliferation stimulated by specific antigen ovalbumin (OVA) is inhibited by mouse and human HLA-DR mAb's ex vivo. g) In vivo efficacy of human HLA-DR mAb's using the mouse model of delayed-type-hypersensitivity (DTH) induced by oxazolone (OXA) as measured by ear-thickness. h) Time course of in vivo efficacy of human HLA-DR mAb 1D09C3 in treating the mouse model of delayed-type-hypersensitivity (DTH) induced by dinitroflurobenzene (DNFB) as measured by ear-thickness. i) Dose response of in vivo efficacy of human HLA-DR mAb 1D09C3 in treating the mouse model of delayed-type-hypersensitivity (DTH) induced by dinitroflurobenzene (DNFB) as measured by ear-thickness.

FIG. 10 Immunosuppressive properties of the IgG forms of the anti-HLA-DR antibody fragments MS-GPC-8-10-57 and MS-GPC-8-27-41 in an assay to determine inhibition of T cell proliferation.

FIG. 11 Vector map and sequence (SEQ ID NO: 33) of scFv phage display vector pMORPH13_scFv. The vector pMORPH13_scFv is a phagemid vector comprising a gene encoding a fusion between the C-terminal domain of the gene III protein of filamentous phage and a HuCAL scFv. In FIG. 11, a vector comprising a model scFv gene (combination of VH1A and Vλ3 (Knappik et al., 2000) is shown. The original HuCAL master genes (Knappik et al. (2000): see FIG. 3 therein) have been constructed with their authentic N-termini: VH1A, VH1B, VH2, VH4 and VH6 with Q (=CAG) as the first amino acid. VH3 and VH5 with E (=GAA) as the first amino acid. Vector pMORPH13_scFv comprises the short FLAG peptide sequence (DYKD) (SEQ ID NO: 33) fused to the VH chain, and thus all HuCAL VH chains in, and directly derived from, this vector have E (=GAA) at the first position (e.g. in pMx7_FS vector, see FIG. 12).

FIG. 12 Vector map and sequence (SEQ ID NO: 34) of scFv expression vector pMx7_FS 5D2. The expression vector pMx7_FS_{—}5D2 leads to the expression of HuCAL scFv fragments (in FIG. 12, the vector comprises a gene encoding a “dummy” antibody fragment called “5D2”) when VH-CH1 is fused to a combination of a FLAG tag (Hopp et al., 1988; Knappik and Plückthun, 1994) and a STREP tag II (WSHPQFEK SEQ ID NO: 34) (IBA GmbH, Göttingen, Germany; see: Schmidt and Skerra, 1993; Schmidt and Skerra, 1994; Schmidt et al., 1996; Voss and Skerra, 1997).

FIG. 13 Vector map and sequence (SEQ ID NO: 35) of Fab expression vector pMx9_Fab_GPC8. The expression vector pMx9_Fab_GPC8 leads to the expression of HuCAL Fab fragments (in FIG. 13, the vector comprises the Fab fragment MS-GPC8) when VH-CH1 is fused to a combination of a FLAG tag (Hopp et al., 1988; Knappik and Plückthun, 1994) and a STREP tag II (WSHPQFEK, SEQ ID No. 8) (IBA GmbH, Göttingen, Germany; see: Schmidt and Skerra, 1993; Schmidt and Skerra, 1994; Schmidt et al., 1996; Voss and Skerra, 1997). In pMx9-Fab vectors, the HuCAL Fab fragments cloned from the scFv fragments (see figure caption of FIG. 11) do not have the short FLAG peptide sequence (DYKD, SEQ ID No. 9) fused to the VH chain, and all HuCAL VH chains in, and directly derived from, that vector have Q (=CAG) at the first position

FIG. 14 Vector map and sequence (SEQ ID NO: 36) of Fab phage display vector pMORPH18_Fab_GPC8. The derivatives of vector pMORPH18 are phagemid vectors comprising a gene encoding a fusion between the C-terminal domain of the gene III protein of filamentous phage and the VH-CH1 chain of a HuCAL antibody. Additionally, the vector comprises the separately encoded VL-CL chain. In FIG. 14, a vector comprising the Fab fragment MS-GPC-8 is shown. In pMORPH18 Fab vectors, the HuCAL Fab fragments cloned from the scFv fragments (see figure caption of FIG. 11) do not have the short FLAG peptide sequence (DYKD, SEQ ID No. 9) fused to the VH chain, and all HuCAL VH chains in, and directly derived from, that vector have Q (=CAG) at the first position.

FIG. 15 Amino acid sequences of VH and VL domains of MS-GPC-1 (SEQ ID NOS 37 and 38, respectively), MS-GPC-6 (SEQ ID NOS 39 and 40, respectively), MS-GPC-8 (SEQ ID NOS 41 and 42, respectively), MS-GPC-10 (SEQ ID NOS 43 and 44, respectively), MS-GPC-8-6 (SEQ ID NOS 41 and 46, respectively), MS-GPC-8-10 (SEQ ID NOS 41 and 48, respectively), MS-GPC-8-17 (SEQ ID NOS 41 and 50, respectively), MS-GPC-8-27 (SEQ ID NOS 41 and 52, respectively), MS-GPC-8-6-13 (SEQ ID NOS 41 and 54, respectively), MS-GPC-8-10-57 (SEQ ID NOS 41 and 56, respectively), MS-GPC-8-27-41 (SEQ ID NOS 41 and 58, respectively), MS-GPC-8-1 (SEQ ID NOS 41 and 28, respectively), MS-GPC-8-9 (SEQ ID NOS 41 and 31, respectively), MS-GPC-8-18 (SEQ ID NOS 41 and 32, respectively), MS-GPC-8-6-2 (SEQ ID NOS 41 and 45, respectively), MS-GPC-8-6-19 (SEQ ID NOS 41 and 47, respectively), MS-GPC-8-6-27 (SEQ ID NOS 41 and 49, respectively), MS-GPC-8-645 (SEQ ID NOS 41 and 51, respectively), MS-GPC-8-647 (SEQ ID NOS 41 and 53, respectively), MS-GPC-8-27-7 (SEQ ID NOS 41 and 55, respectively), and MS-GPC-8-27-10 (SEQ ID NOS 41 and 57, respectively). The sequences in FIG. 15 show amino acid 1 of VH as constructed in the original HuCAL master genes (Knappik et al. (2000): see FIG. 3 therein). In scFv constructs, as described in this application, amino acid 1 of VH is always E (see figure caption of FIG. 11), in Fab constructs as described in this application, amino acid 1 of VH is always Q (see figure caption of FIG. 13).

FIG. 16. In vivo effect of the human anti-DR mAb 1D09C3 in lymphoma xenograft models. a) survival of SCID mice injected s.c. with the non-Hodgkin lymphoma line GRANTA-519. MAb dose was 3×1 mg/mouse given on days 5, 7, and 9. Seven mice in the control and five in each mAb treated group. b) Effect of mAb on subcutaneous tumor growth. Same experiment as in a. c) Effect of mAb on disease incidence in SCID mice injected i.v. with GRANTA-519. MAb was administered i.v, 3× as above. Six mice were in each group.

FIG. 17. The mAb 1D09C3 in a dose response experiments in a Non-Hodgkin's Lymphoma Model (Granta-519). The mAb 1D09C3 exhibits comparable efficacy within a does range of 1 mg to 2.5 μg/mouse (50 mg to 125 μg/kg). Efficacy titrates between 2.5 μg (full efficacy) and 25 ng/mouse (no detectable efficacy).

FIG. 18. Combination of 1D09C3 and Rituxan in Non-Hodgkin's Lymphoma (NHL) Model (Granta-519). The anti-HLA-DR mAb 1D09C3 shows a clear synergism with the anti-CD20 mAb Rituxan in an NHL model. Single therapies with each antibody show comparable efficacies.

FIG. 19. Efficacy in different xenotransplant models. The 1D09C3 mAb is effective in xenotransplant models of Hodgkin's lymphoma, non-Hodgkin's lymphoma, multiple myeloma and hairy cell leukemia.

FIG. 20. Killing of Melanoma cell lines. The 1D09C3 mAb exhibits comparable efficacy within a dose range of 1 mg to 2.5 μg/mouse (50 mg to 125 μg/kg) In addition to malignant lymphoid cells, 1D09C3 can induce cell death also in non-lympoid solid tumors, as evidenced by killing of HLA-DR+melanoma cells in vitro.

FIG. 21. Late treatment of disseminated Lymphoma with the 1D09C3 mAb. In a model of terminal stage disease (˜7 days before moribund, histologically characterized as disseminated lymphoma in multiple organs), 1D09C3 could still rescue 33% of treated animals.

FIG. 22. Schematic representation of known signaling events and pathological changes occurring after treatment of activated/tumor transformed cells with an apoptotic anti-MHC-II antibody. Applicants present the schematic representation here for illustration purpose only, and without wish to be bound by the representation.

### DETAILED DESCRIPTION OF THE INVENTION

The following examples illustrate the invention.

### EXAMPLES

All buffers, solutions or procedures without explicit reference can be found in standard textbooks, for example Current Protocols of Immunology (1997 and 1999) or Sambrook et al., 1989. Where not given otherwise, all materials were purchased from Sigma, Deisenhofen, Del., or Merck, Darmstadt, Del., or sources are given in the literature cited. Hybridoma cell lines LB3.1 and L243 were obtained from LGC Reference Materials, Middlesex, UK; data on antibody 8D1 were generously supplied by Dr. Matyas Sandor, University of Michigan, Madison, Wis., USA.

1. Preparation of a Human Antigen

To demonstrate that we could identify cytotoxic human antigen-binding domains, we first prepared a purified form of a human antigen, the human MHC class II DR protein (DRA*0111/DRB1*0401) from the DR-homozygous B-lymphoblastoid line PRIESS cells (Gorga et al., 1984; Gorga et al., 1986; Gorga et al., 1987; Stern et al., 1992) and the human-mouse chimeric molecule DR-I_{E} from the transfectant M12.C3.25 (Ito et al., J. Exp. Med. 183:2635-2644, 1996) by using standard methods of affinity purification (Gorga et al., 1984) as follows.

First, PRIESS cells (ECACC, Salisbury UK) were cultured in RPMI and 10% fetal calf serum (FCS) using standard conditions, and 10¹⁰ cells were lysed in 200 ml phosphate buffered saline (PBS) (pH 7.5) containing 1% NP-40 (BDH, Poole, UK), 25 mM iodoacetamide, 1 mM phenylmethylsulfonylfluoride (PMSF) and 10 mg/L each of the protease inhibitors chymostatin, antipain, pepstatin A, soybean trypsin inhibitor and leupeptin. The lysate was centrifuged at 10,000×g (30 minutes, 4° C.) and the resulting supernatant was supplemented with 40 ml of an aqueous solution containing 5% sodium deoxycholate, 5 mM iodoacetamide and 10 mg/L each of the above protease inhibitors and centrifuged at 100,000×g for two hours (4° C.). To remove material that bound non-specifically and endogenous antibodies, the resulting supernatant was made 0.2 mM with PMSF and passed overnight (4° C.) through a rabbit serum affigel-10 column (5 ml; for preparation, rabbit serum (Charles River, Wilmington, Mass., USA) was incubated with Affigel 10 (BioRad, Munich, Del.) at a volume ratio of 3:1 and washed following manufacturer's directions) followed by a Protein G Sepharose Fast Flow column (2 ml; Pharmacia) using a flow rate of 0.2 ml/min.

Second, the pre-treated lysate was batch incubated with 5 ml Protein G Sepharose Fast Flow beads coupled to the murine anti-LILA-DR antibody LB3.1 (obtained by Protein G-Sepharose FF (Pharmacia) affinity chromatography of a supernatant of hybridoma cell line LB3.1) (Stern et al., 1993) overnight at 4° C. using gentle mixing, and then transferred into a small column which was then washed extensively with three solutions: (1) 100 ml of a solution consisting of 50 mM Tris/HCl (pH 8.0), 150 mM NaCl, 0.5% NP-40, 0.5% sodium deoxycholate, 10% glycerol and 0.03% sodium azide at a flow rate of 0.6 ml/min). (2) 25 ml of a solution consisting of 50 mM Tris/HCl (pH 9.0), 0.5 M NaCl, 0.5% NP-40, 0.5% sodium deoxycholate, 10% glycerol and 0.03% sodium azide at a flow rate of 0.9 ml/min; (3) 25 ml of a solution consisting of 2 mM Tris/HCl (pH 8.0), 1% octyl-β-D-glucopyranoside, 10% glycerol and 0.03% sodium azide at a flow rate of 0.9 ml/min.

Third, MHC class II DR protein (DRA*0101/DRB1*0401) was eluted using 15 ml of a solution consisting of 50 mM diethylamine/HCl (pH 11.5), 150 mM NaCl, 1 mM EDTA, 1 mM EGTA, 1% octyl-B-D-glucopyranoside (Alexis Corp., Lausen, C H), 10% glycerol, 10 mM iodoacetamide and 0.03% sodium azide at a flow rate of 0.4 ml/min. 800 μl fractions were immediately neutralised with 100 μl 1M Tris/HCl (pH 6.8), 150 mM NaCl and 1% octyl-B-D-glucopyranoside. The incubation of the lysate with LB3.1-Protein G Sepharose Fast Flow beads was repeated until the lysate was exhausted of MHC protein. Pure eluted fractions of the MHC class I DR protein (as analyzed by SDS-PAGE) were pooled and concentrated to 1.0-1.3 g/L using Vivaspin concentrators (Greiner, Solingen, Del.) with a 30 kDa molecular weight cut-off. Approximately 1 mg of the MHC class U DR preparation was re-buffered with PBS containing 1% octyl-o-D-glucopyranoside using the same Vivaspin concentrator to enable direct coupling of the protein to BIAcore CM5 chips.

2. Screening of HuCAL

2.1. Introduction

Since the important biological activities of anti-DR mAbs, e.g., inhibition of CD4 T cell—antigen presenting cell (APC) interaction and tumoricide activity are associated with specificity for the first, N-terminal domains of DR molecules (Vidovic, D. et al., 1995, Eur. J. Immunol. 25:3349-3355), we used purified DR molecules as well as human-murine chimeric MHC-II molecules (DR first domains grafted onto a murine class II molecule, see Ito, K. et al., 1996, J. Exp. Med. 183:2635-2644) for screening the Human Combinatorial Antibody Library (HuCAL®) by alternating whole cell panning with protein solid-phase-panning.

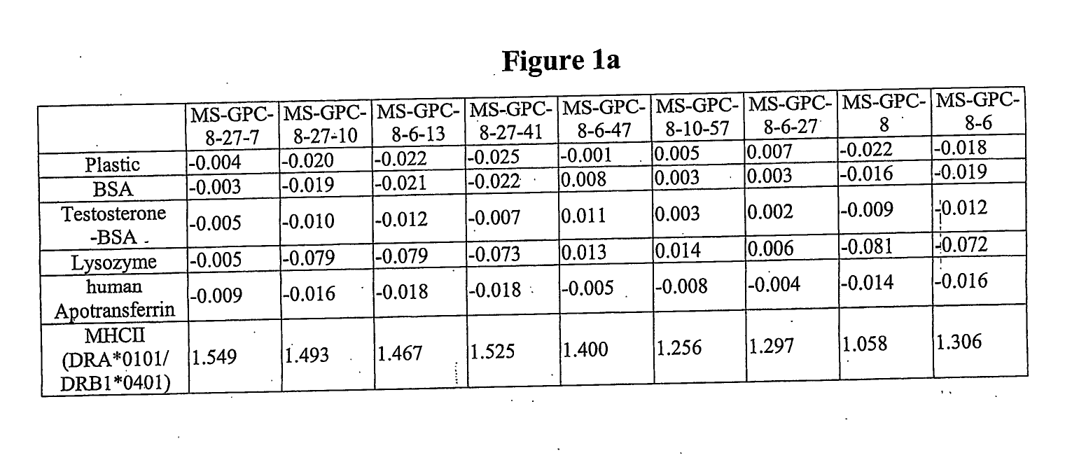

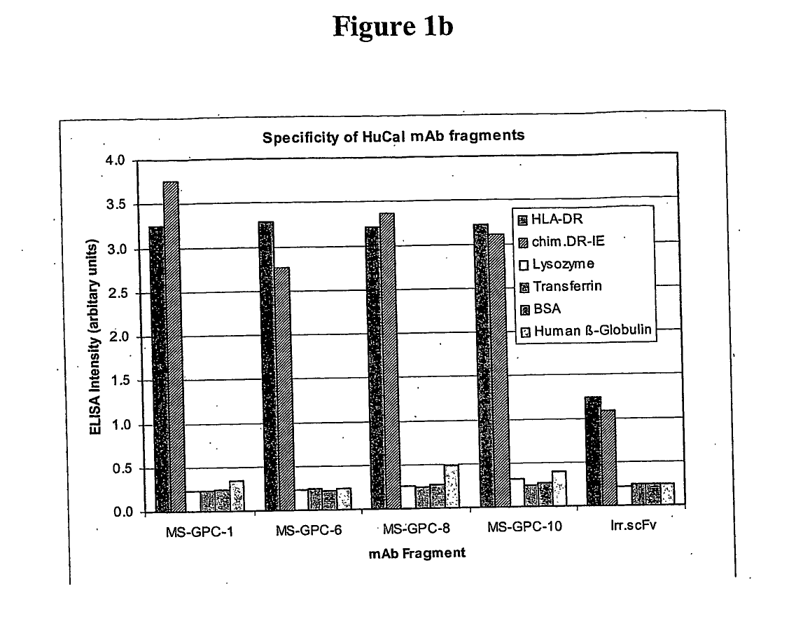

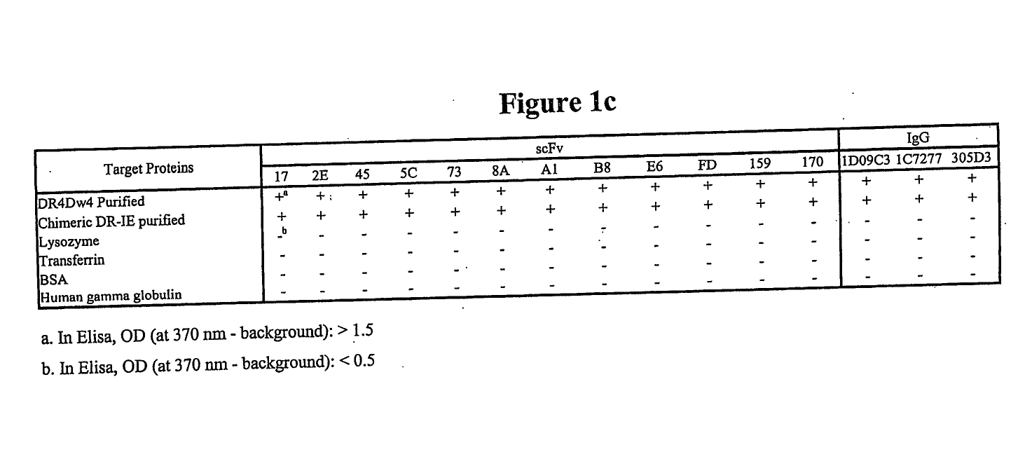

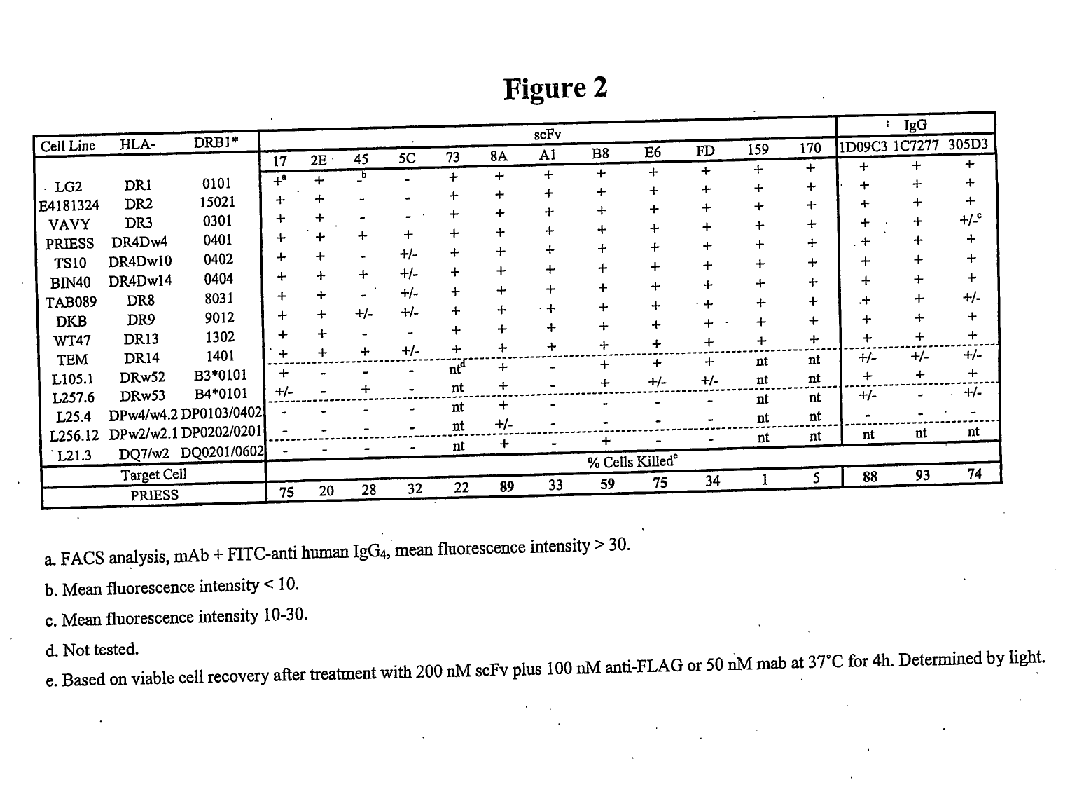

We identified certain human antigen binding antibody fragments (in this case, scFvs) (MS-GPC-1/scFv-17, MS-GP-6/scFv-8A, MS-GPC-8/scFv-B8, MS-GPC-10/scFv-E6, etc., see FIGS. 1 and 2) against the human antigen (DRA*0101/DRB1*0401) from a human antibody library based on a novel concept that has been recently developed (Knappik et al., 2000). A consensus framework resulting in a total of 49 different frameworks here represents each of the VH- and VL-subfamilies frequently used in human immune responses. These master genes were designed to take into account and eliminate unfavorable residues promoting protein aggregation as well as to create unique restriction sites leading to modular composition of the genes. In HuCAL-scFv, both the VH- and VL-CDR3 encoding regions of the 49 master genes were randomized.

2.2. Phagemid Rescue, Phase Amplification and Purification

The HuCAL-scFv (Knappik et al., 2000) library, cloned into a phagemid-based phage display vector pMORPH13_scFv (see FIG. 1), in E. coli TG-1 was amplified in 2×TY medium containing 34 μg/ml chloramphenicol and 1% glucose (2×TY-CG). After helper phage infection (VCSM13) at 37° C. at an OD₆₀₀ of about 0.5, centrifugation and resuspension in 2×TY/34 μg/ml chloramphenicol/50 μg/ml kanamycin/0.1 mM IPTG, cells were grown overnight at 30° C. Phage were PEG-precipitated from the supernatant (Ausubel et al., 1998), resuspended in PBS/20% glycerol and stored at −80° C. Phage amplification between two panning rounds was conducted as follows: mid-log phase TG1-cells were infected with eluted phage and plated onto LB-agar supplemented with 1% of glucose and 34 μg/ml of chloramphenicol. After overnight incubation at 30° C. colonies were scraped off, adjusted to an OD₆₀₀ of 0.5 and helper phage added as described above.

2.3. Manual Solid Phase Panning

Wells of MaxiSorp™ microtiterplates (Nunc, Roskilde, DK) were coated with MHC-class II DRA*0101/DRB1*0401 (prepared as above) dissolved in PBS (2 μg/well). After blocking with 5% non-fat dried milk in PBS, 1-5×10¹² HuCAL-scFv phage purified as above were added for 1 h at 20° C. After several washing steps, bound phages were eluted by pH-elution with 100 mM triethylamine and subsequent neutralization with 1 M Tris-Cl pH 7.0. Three rounds of panning were performed with phage amplification conducted between each round as described above.

2.4. Mixed Solid Phase/Whole Cell Panning

Three rounds of panning and phage amplification were performed as described in 2.3. and 2.2. with the exception that in the second round between 1×10⁷ and 5×10⁷ PRIESS cells in 1 ml PBS/10% FCS were used in 10 ml Falcon tubes for whole cell panning. After incubation for 1 h at 20° C. with the phage preparation, the cell suspension was centrifuged (2,000 rpm for 3 min) to remove non-binding phage, the cells were washed three times with 10 ml PBS, each time followed by centrifugation as described. Phage that specifically bound to the cells were eluted off by pH-elution using 100 mM HCl. Alternatively, binding phage could be amplified by directly adding E. coli to the suspension after triethlyamine treatment (100 mM) and subsequent neutralization.

2.5 Identification of HLA-DR Binding scFv Fragments

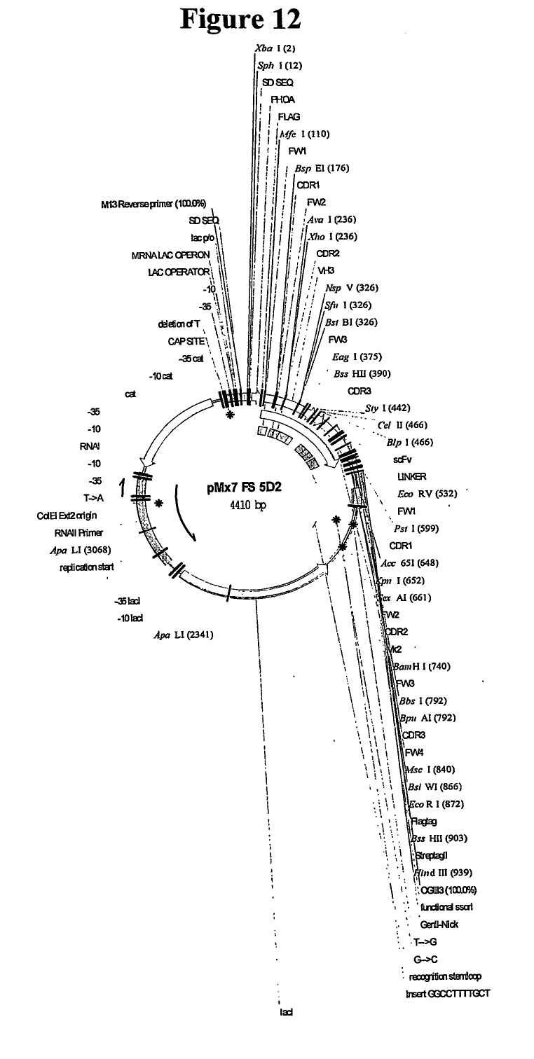

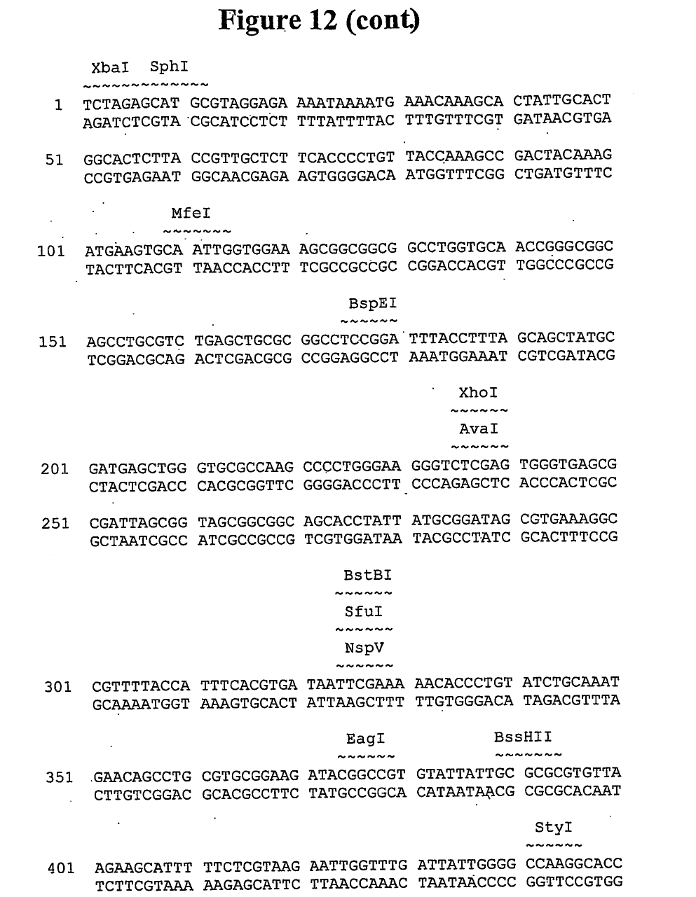

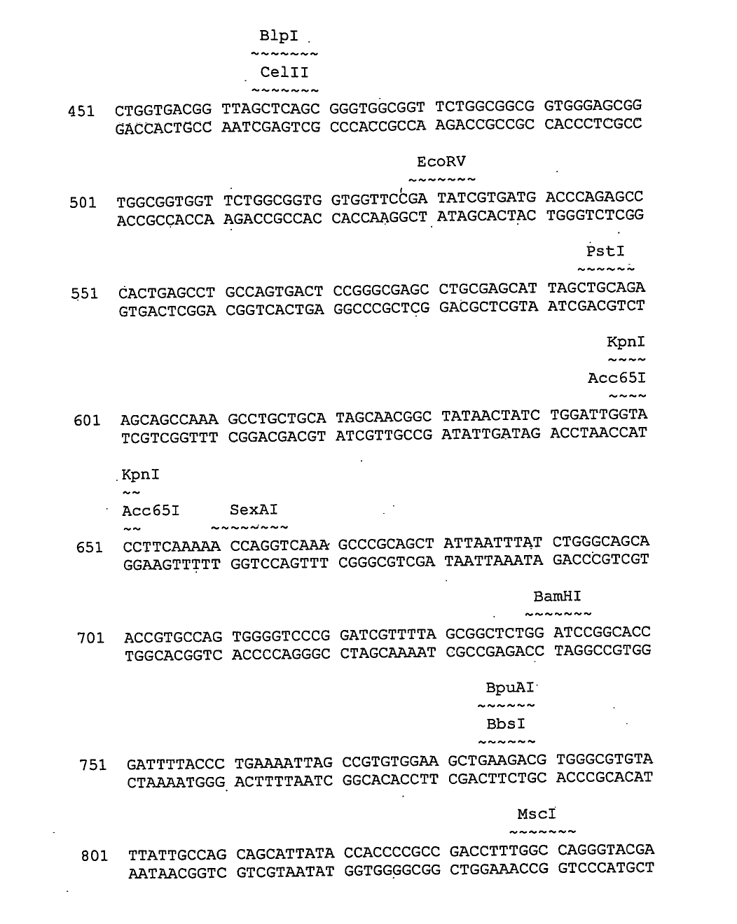

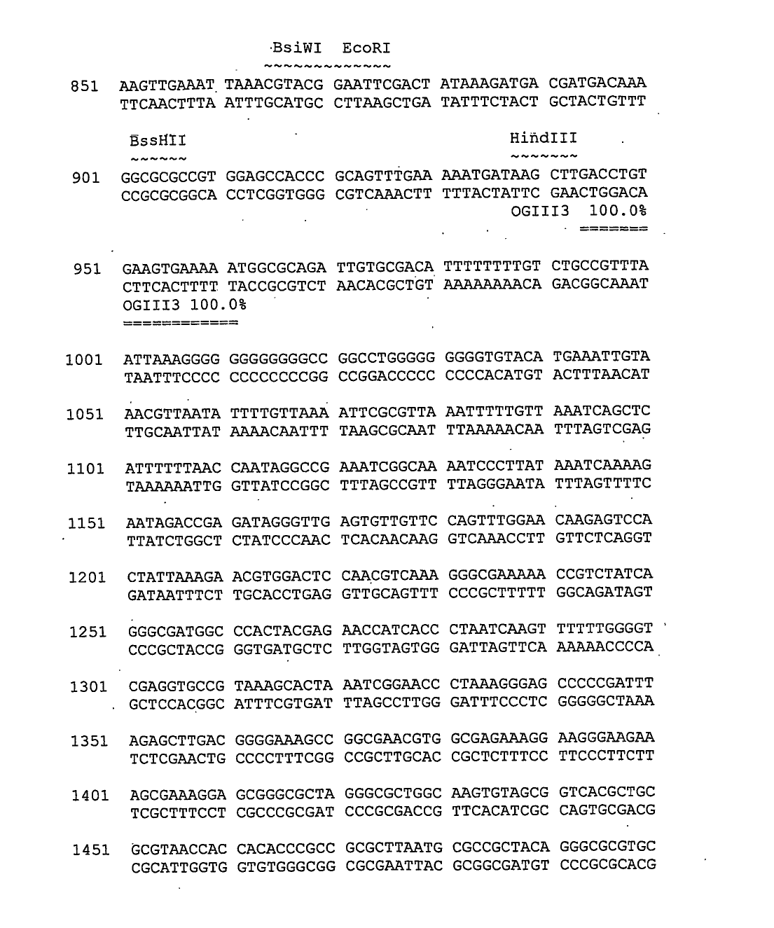

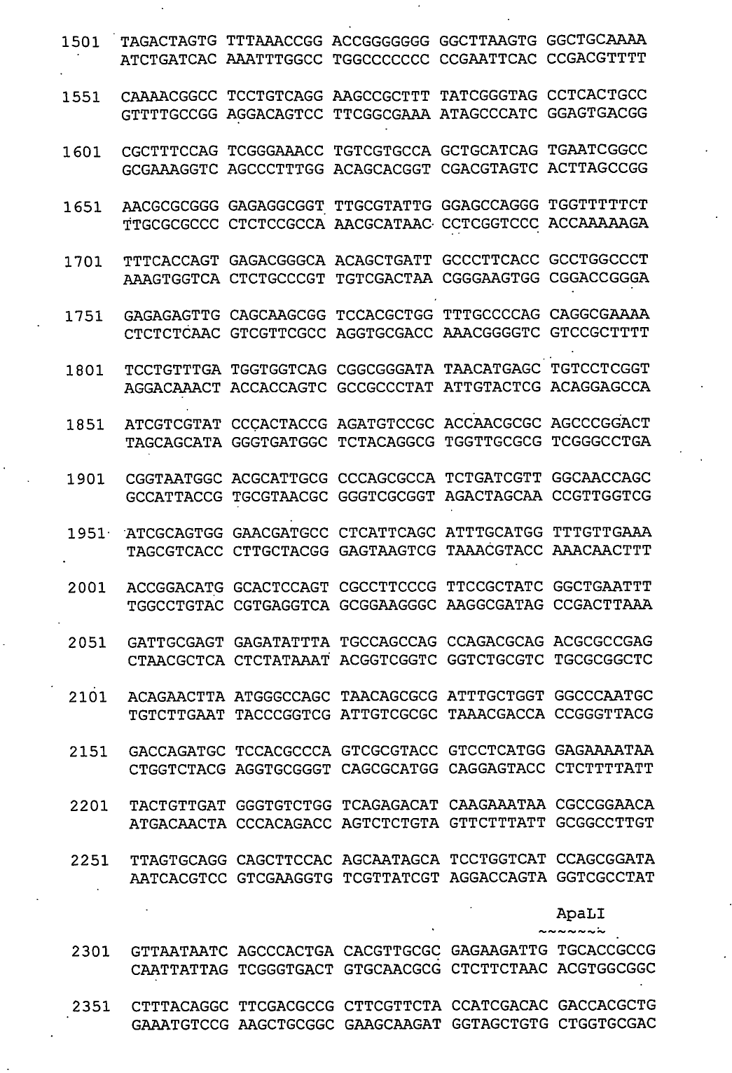

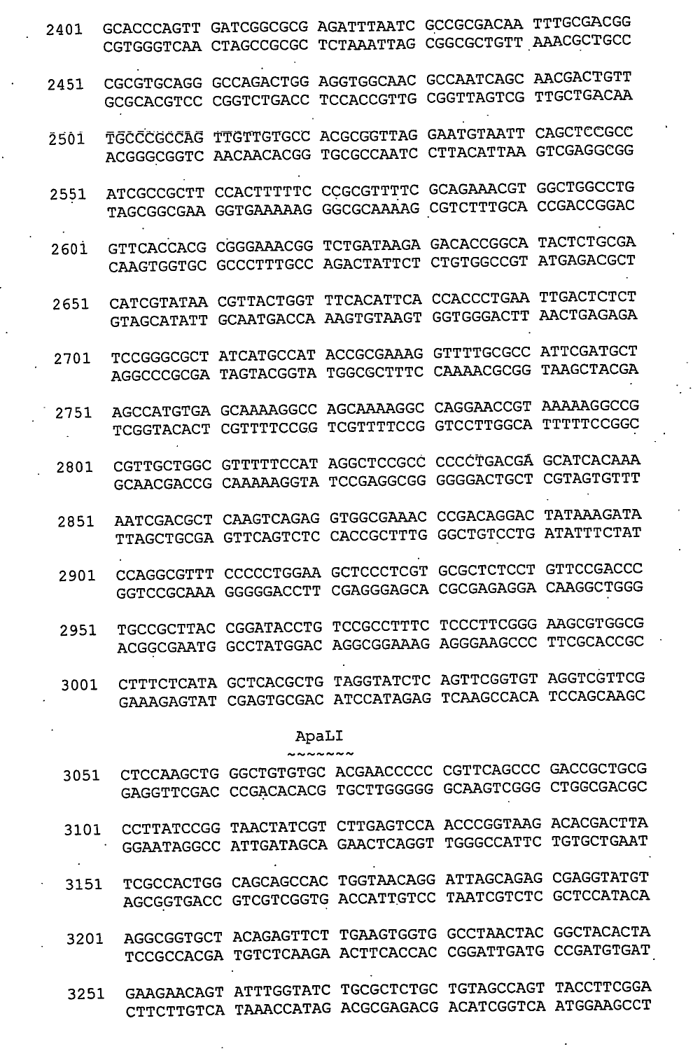

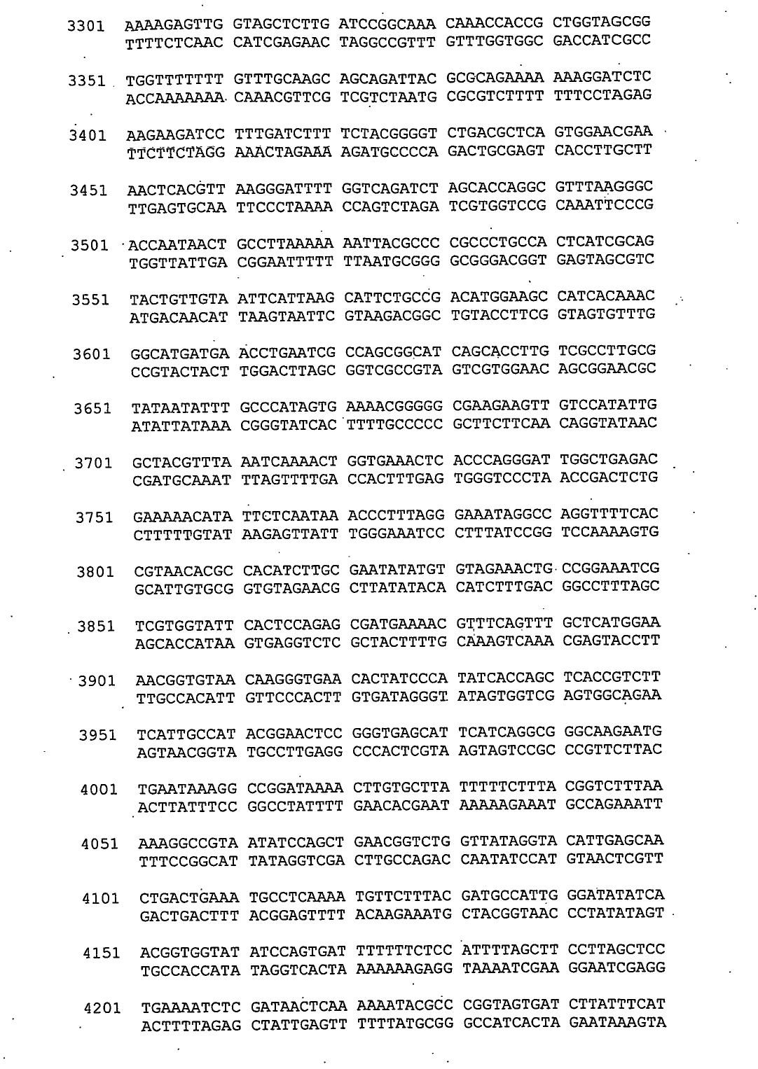

Clones obtained after three rounds of solid phase panning (2.3) or mixed solid phase/whole cell panning (2.4) were screened by FACS analysis on PRIESS cells for binding to HLA-DR on the cell surface. For expression, the scFv fragments were cloned via XbaI/EcoRI into pMx7_FS as expression vector (see FIG. 12). Expression conditions are shown below in example 3.2.

Aliquots of 10⁶ PRIESS cells were transferred at 4° C. into wells of a 96-well microtiterplate. ScFv in blocking buffer (PBS/5% FCS) were added for 60 min and detected using an anti-FLAG M2 antibody (Kodak) (1:5000 dilution) followed by a polyclonal goat anti-mouse IgG antibody-R-Phycoerythrin-conjugate (Jackson ImmunoResearch, West Grove, Pa., USA, Cat. No. 115-116-146, F(ab′)₂ fragment) (1:200 dilution). Cells were fixed in 4% paraformaldehyde for storage at 4° C. 104 events were collected for each assay on the FACS-Calibur (BD Immunocytometry Systems, San Jose, Calif., USA).

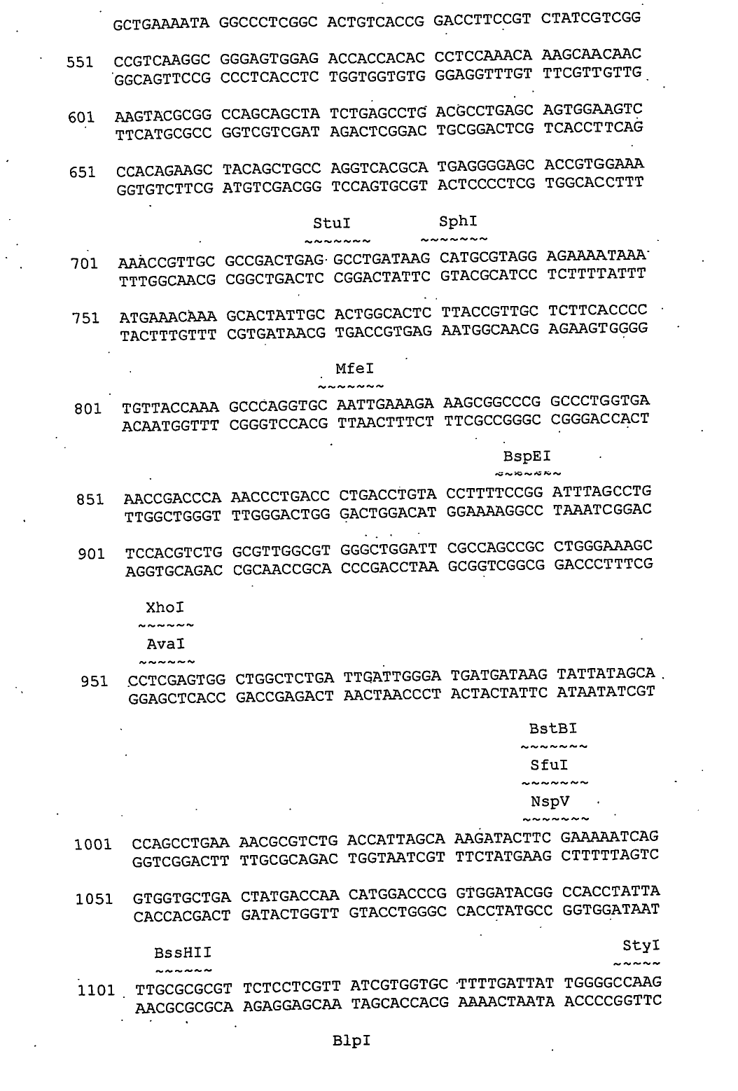

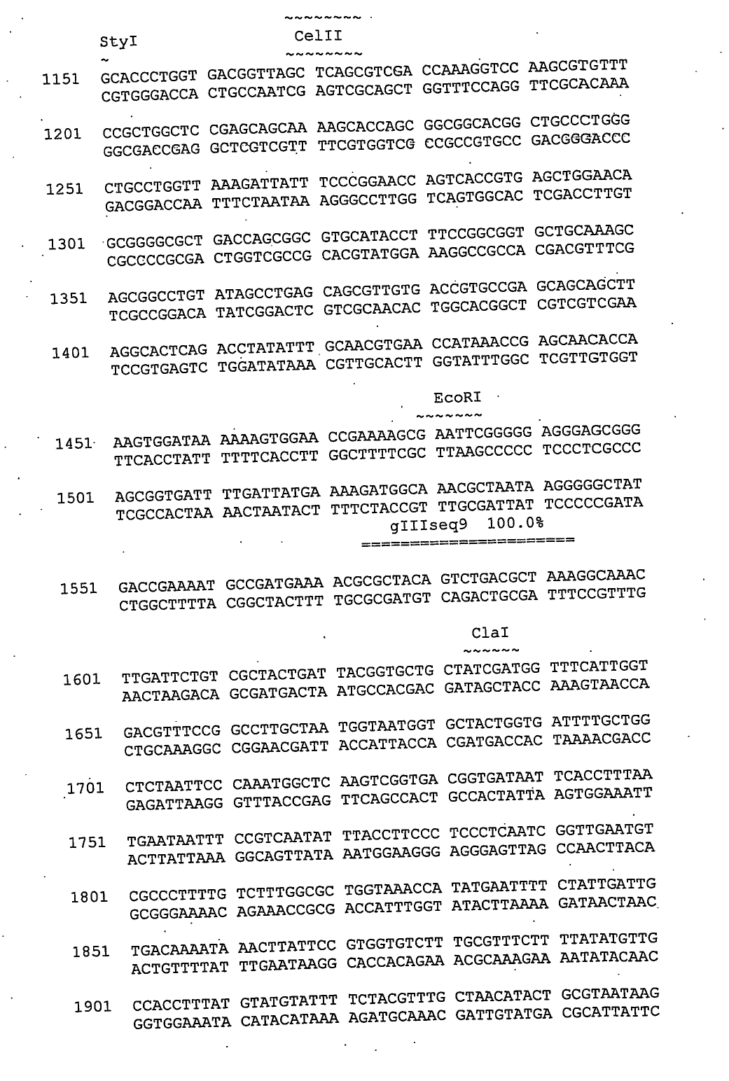

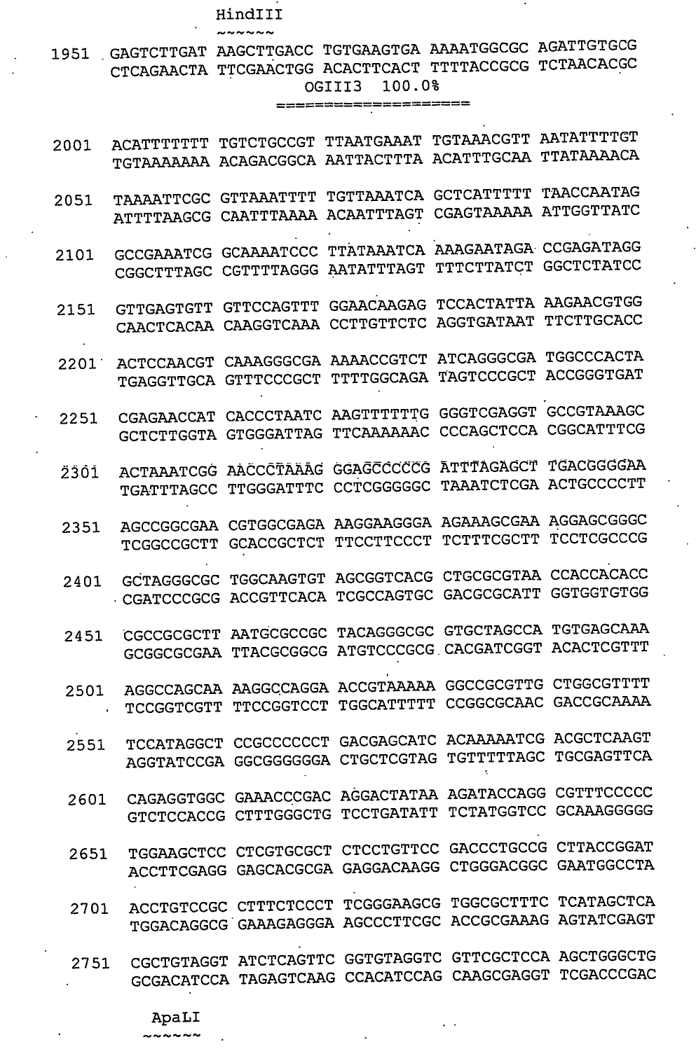

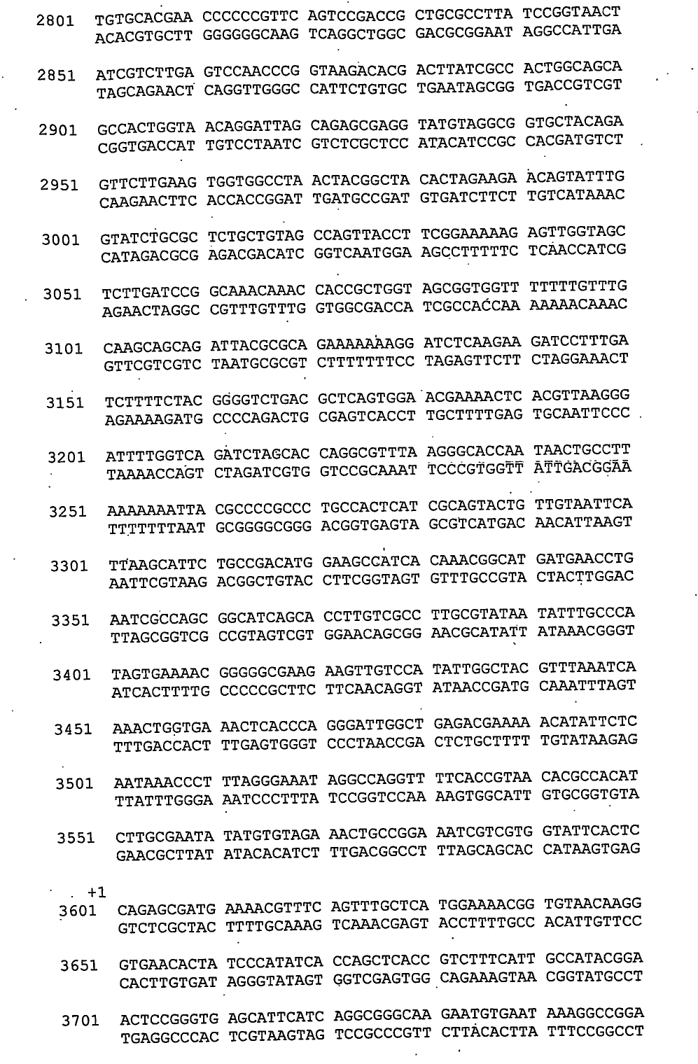

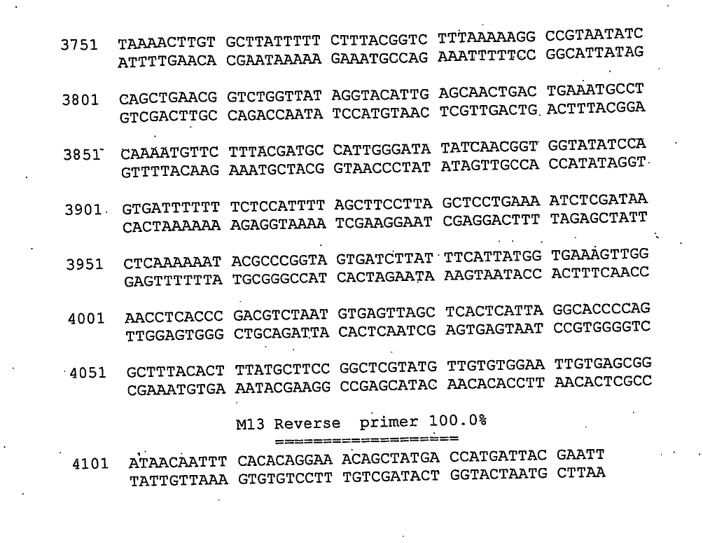

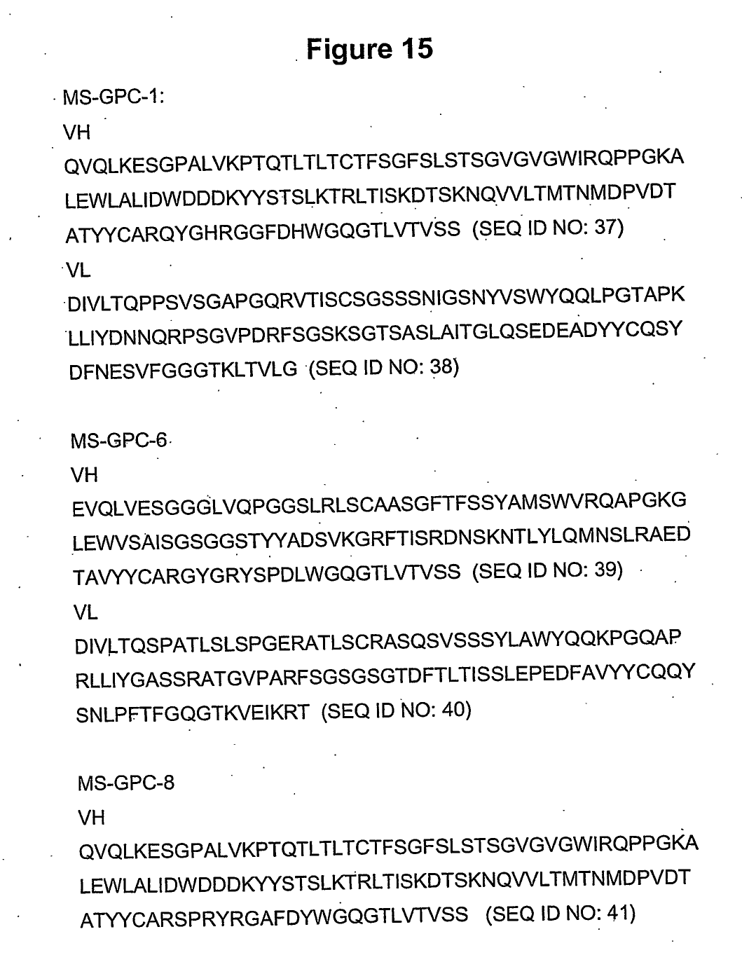

Only fifteen out of over 500 putative binders were identified which specifically bound to PRIESS cells. Twelve scFv-s also bound to the chimeric MHC-II molecule, but showed no reactivity to either I-E^{d} (the murine part of chimeric MHC-II27), or unrelated proteins, such as lysozyme, transferrin, bovine serum albumine and human gamma globuline (FIG. 1), indicating that they were specific for the first domains of DR molecules. Some of these clones were further analysed for their immunomodulatory ability and for their killing activity as described below. Table 1 contains the sequence characteristics of clones MS-GPC-1 (scFv-17), MS-GPC-6 (scFv-8A), MS-GPC-8 (scFv-B8) and MS-GPC-10 (scFv-E6) identified thereby. The VH and VL families and the CDR3s listed refer to the HuCAL consensus-based antibody genes as described (Knappik et al., 2000); the sequences of the VH and VL CDRs are shown in Table 1, and the full sequences of the VH and VL domains are shorn in FIG. 15;

The fine specificity of scFv-s was tested on a panel of DR-homozygous typing cells, and MHC-II transfectants. Ten of 12 scFv-s reacted with all major allelic forms of DR represented in the cell panel (DR1 through 14), and 4 of 12 recognized additional MHC-II molecules (DRw52 and w53, DP and DQ molecules; FIG. 2). Thus, these antibodies potentially could be used-widely as therapeutic agents across human populations virtually irrespective of polymorphic differences in MHC-II molecules. Most importantly, four of the 12 hits exhibited strong tumor killing activity, when cross-linked with anti-tag antibody (see FIG. 2, in bold). The monovalent fragments were not tumoricidal, corresponding to previous observations (Vidovic', D. et al., 1995, Eur. J. Immunol. 25:3349-3355).

3. Generation of Fab-Fragments

3.1. Conversion of scFv to Fab

Since the tumoricidal hits had modest affinities (K_{d}-s ranging from 346 nM to 81 μM in single chain Fv (scFv) format), they were subjected to “in vitro affinity maturation”. The parental scFv-s were first converted into Fab format that is less prone to aggregation and hence should give more reliable K_{off} values.

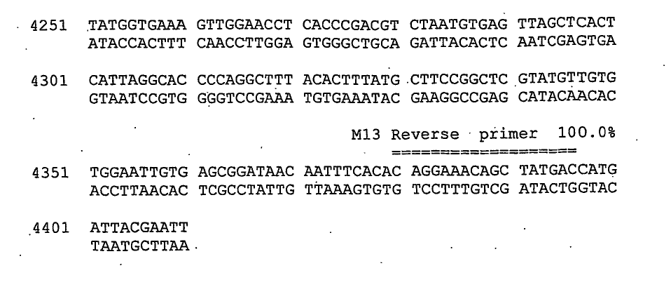

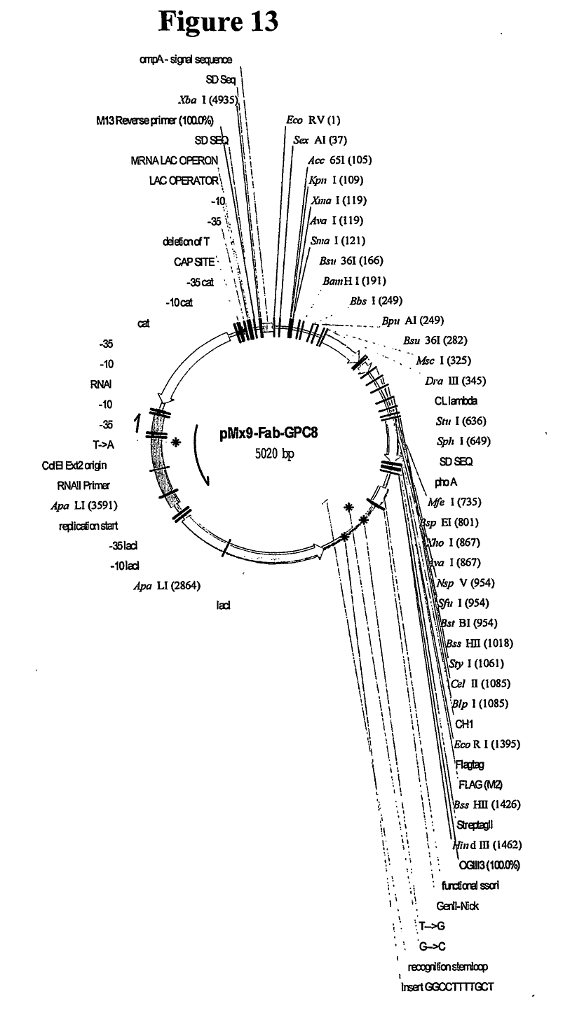

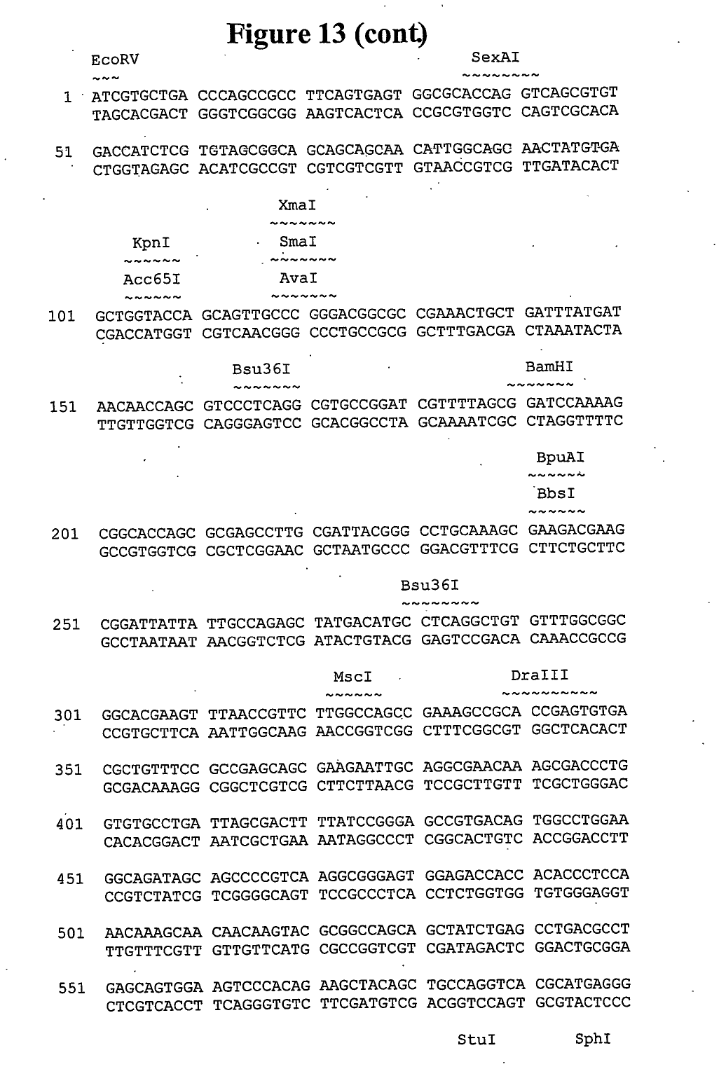

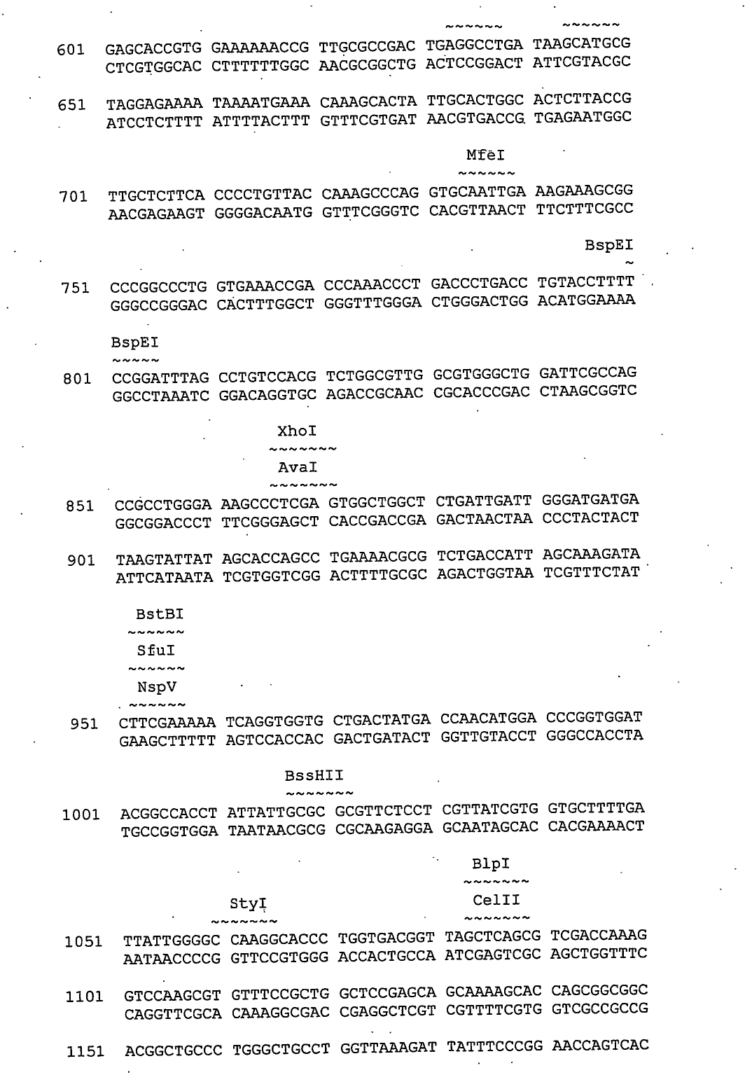

The Fab-fragment antigen binding polypeptides MS-GPC-1-Fab/17-Fab, MS-GP-6-Fab/8A-Fab, MS-GPC-8-Fab/B8-Fab and MS-GPC-10-Fab/E6-Fab were generated from their corresponding scFv fragments as follows. Both heavy and light chain variable domains of scFv fragments were cloned into pMx9 Fab (FIG. 13), the heavy chain variable domains as MfeI/StyI-fragments, the variable domains of the kappa light chains as EcoRV/BsiWI-fragments. The lambda chains were first amplified from the corresponding pMORPH13_scFv vector as template with PCR-primers CRT5 (5′ primer) and CRT6 (3′ primer), wherein CRT6 introduces a unique DraIII restriction endonuclease site.

CRT5 (SEQ ID No. 10):
5′ GTGGTGGTTCCGATATC 3′
CRT6 (SEQ ID No. 11):
5′ AGCGTCACACTCGGTGCGGCTTTCGGCTGGCCAAGAACGGGTTA 3′

The PCR product is cut with EcoRV/DraIII and cloned into pMx9 Fab (see FIG. 13). The Fab light chains could be detected with a polyclonal goat anti-human IgG antibody-R-Phycoerythrin-conjugate (Jackson ImmunoResearch, West Grove, Pa., USA, Cat. No. 109-116-088, F(ab′)₂ fragment) (1:200 dilution).

3.2. Expression and Purification of HuCAL-Antibody Fragments in E. coli

Expression in E. coli cells (JM83) of scFv and Fab fragments from pMx7_FS or pMx9 Fab, respectively, were carried out in one litre of 2×TY-medium supplemented with 34 μg/ml chloramphenicol. After induction with 0.5 mM IPTG (scFv) or 0.1 mM IPTG (Fab), cells were grown at 22° C. for 12 hours. Cell pellets were lysed in a French Press (Thermo Spectronic, Rochester, N.Y., USA) in 20 mM sodium phosphate, 0.5 M NaCl, and 10 mM imidazole (pH 7.4). Cell debris was removed by centrifugation and the clear supernatant filtered through 0.2 μm pores before subjecting it to STREP tag purification using a Streptactin matrix and purification conditions according to the supplier (IBA GmbH, Göttingen, Germany). Purification by size exclusion chromatography (SEC) was performed as described by Rheinnecker et al. (1996). The apparent molecular weights were determined by SEC with calibration standards and confirmed in some instances by coupled liquid chromatography-mass spectrometry (TopLab GmbH, Martinsried, Germany).

4. Optimization of Antibody Fragments

In order to optimize certain biological characteristics of the HLA-DR binding antibody fragments, one of the Fab fragments, MS-GPC-8-Fab/B8-Fab, was used to construct a library of Fab antibody fragments by replacing the parental VL λ1 chain by the pool of all lambda chains λ1-3 randomized in CDR3 from the HuCAL library (Knappik et al., 2000).

In the first round of optimization, both H-CDR2- and L-CDR3-sequences of clones MS-GPC-1/scFv-17, MS-GPC-6/scFv-8A, MS-GPC-8/scFv-B8 and MS-GPC-10/scFv-E6 were randomized by substituting the parental sequence with randomized TRIM®-based oligonucleotide-cassettes (Virnekäs et al., 1994) leading to four different libraries with 7.6×10⁶ to 1.0×10⁷ primary transformants.

For generation of H-CDR2 and L-CDR1-libraries: Trinucleotide-containing oligonucleotides starting from O-methyl trinucleotide phosphoramidites (Virnekäs 1994) were synthesized as described (Knappik et al., 2000). The VH2-CDR2-design comprised an olionucleotide encoding for 16 amino acids which was randomized with up to 19 different amino acids (all except for cystein) at the following positions (from N- to C-terminus; amino acid-diversity and ratios in % are given in parentheses): position-1 (19), -2 (40% V/20% D, F, N), -3 (40% V/20% D, V, N), -4 (19), -5 (19), -6 (D), -7 (19), -8 (K), -9 (19), -10 (Y), -11 (70% S/30% G), -12 (50% P/50% T), -13 (S), -14 (L), -15 (K), -16 (S). For the L-CDR1 of the lambda-1-framework two different oligonucleotides (termed as a and b) were designed to encode: a) position-1 (S), -2 (G), -3 (S), -4 (19); -5 (S), -6 (80% N/10% D, K), -7 (I), -8 (G), -9 (19), -10 (19), -11 (19), -12 (V), -13 (19); b) position 1 (50% S, T), -2 (G), -3 (S); -4 (80% S/20% N), -5 (S), 6 (N), -7 (I), -8 (G), -9 (19), -10 (19), -11 (19), -12 (19), -13 (V), -14 (19). The oligonucleotide for the CDR1 of lambda-2 framework was designed to encode: position-1 (19), -2 (G), -3 (S), 4 (89% S/20% T), -5 (S), -6 (D), -7 (80% V, 20% I), -8 (G), )-9 (19), -10 (Y), -11 (19), -12 (19), -13 (V), -14 (19). For framwork lambda 3 the following CDR1-design was made: position-1 (33% G, Q, S), -2 (G), -3 (50% D, N), 4 (19), -5 (50% L, I), -6 (33% G, P, R), -7 (19), -8 (19), -9 (19), -10 (50% A, V), -11 (19). All cassettes were introduced into a promoter-less derivative of pMorph4 (Pack et al., in preparation). For all subsequent affinity-maturations the respective H-CDR2 or L-CDR1-cassettes were derived from those plasmids using the respective flanking restriction-nuclease sites as described (Knappik et al., 2000). Prior to cloning of different libraries for affinity Maturation all parental scFv were converted into the Fab-format following the standard conversion protocol (Krebs et al., 2001) for the modular HuCAL-library. Based on each of the 4 parental Fabs 17, 8A, B8 and E6 (all H2 lambda1) a sub-library was constructed exhibiting a repertoire of different L-CDR3- and H-CDR2-cassettes. First cloning step included the substation of the parental XbaI/DraIII-fragment of Fabs 17, B8, and E6 by a mix of corresponding fragments of all 3 V lambda consensus-genes encoding a repertoire of 5.7×10⁶ different L-CDR3 cassettes. Library-sizes for all 3 parental clones were in the range of 5.1-6.0×10⁶ transformants. These libraries were then used to introduce different H-CDR2-library cassettes via substitution of the XhoI/EagI-fragments. Final library sizes resulted in up to 1.2×10⁷ transformants including 78% correct clones based on DNA-sequence analysis. In case of 8A the LCDR3 optimization was performed by exchanging the parental XbaI/BsiWI-fragment for the corresponding HuCAL-scFv kappa3 sublibrary fragments. As before, this library was then used to insert different HCDR2-cassettes via the Xho/BssHII-fragment. Library sizes were in the range of 1.7×10⁶ cfu after L-CDR3- and 1.0×10⁷ cfu after H-CDR2-cassette insertion including at least 65% correct clones according to DNA-sequence analysis. A fifth library has been constructed based on a consensus-sequence within H-CDR3 of binders 17, B8 and E6. For this purpose parental Fab B8 has been chosen to randomize several positions within H-CDR3 by insertion of a synthetic TRIM-oligonucleotide comprising the following H-CDR3-design from N- to C-terminus: position 1 (all=all exept C), -2 (all), -3 (all), -4 (25% of Y/W/F/H), -4 (R), -5 (G), -6 (50% G/A), -7 (50% F/L), -8 (all). Final library size was in the range of 6.8×10⁶ different transformants comprising 63% correct clones after sequence analysis.

L-CDR1-libraries were generated based on a pool of 20 different Fab-clones derived from the combined light-chain- and H-CDR2-based-optimization. Equimolar, amounts of vector DNA from each parental clone was mixed after removal of the EcoRV/BpuAI-insert and religated by insertion of the corresponding fragments encoding a repertoire of different L-CDR1-cassettes. Final library-sizes were in the range of 4.2×10⁸ cfu.

Since clones 17, B8 and E6 exhibited a consensus-motif in H-CDR3, a fifth library was constructed based on the parental clone B8, in which several H-CDR3 positions were randomized while keeping the consensus motif constant. The latter library termed B8M gave rise to 6.8×10⁶ initial transformants. AU libraries were subjected to either two rounds of standard solid-phase panning on purified DR, or a solid phase and a whole cell panning.

Several panning-parameters including decreasing amounts of antigen (500 ng and 250 ng/well purified protein, see Schier et al., 1996a and 1996b), or increasing concentrations of NH₄SCN (50 mM, 250 mM, 500 mM in PBS) (Hall and Heckel 1988; MacDonald 1988; Goldblatt 1993; Ferreira & Katzin 1995), or increasing the numbers of wash-cycles (Chen 1999; Low 1996) were applied in the second panning-round to enhance panning-stringency and hence the probability of selecting high affinity Fabs. Phage-antibodies derived from the first round of a manual solid-phase-panning on 250 and 500 ng/well purified HLA-protein, respectively, were pooled and used for the second panning round on either 12 ng/well purified protein according to a standard protocol (Krebs et al., 2001), or on 250 ng coated antigen in combination with an additional 30 min incubation-step of different amounts of ammonium-isothiocyanate (50 mM, 250 mM, 500 mM and in PBS) in between the standard wash-protocol (5×TBST short and 5×TBST for 5 min at room temperature) and the elution step (100 mM glycine-HCl/500 mM NaCl, pH 2.2). Alternatively, the second panning round was performed on different amounts of PRIESS-cells ranging from 10¹-10⁵ cells/well according to a standard whole-cell-panning-protocol (Krebs et al., 2001). Fab-clones for K_{off} rankings were selected only from those panning wells which prior to and after treatment show a significant drop in phage-titer and thus indicating a maximum in bound phages at the highest panning-stringency.

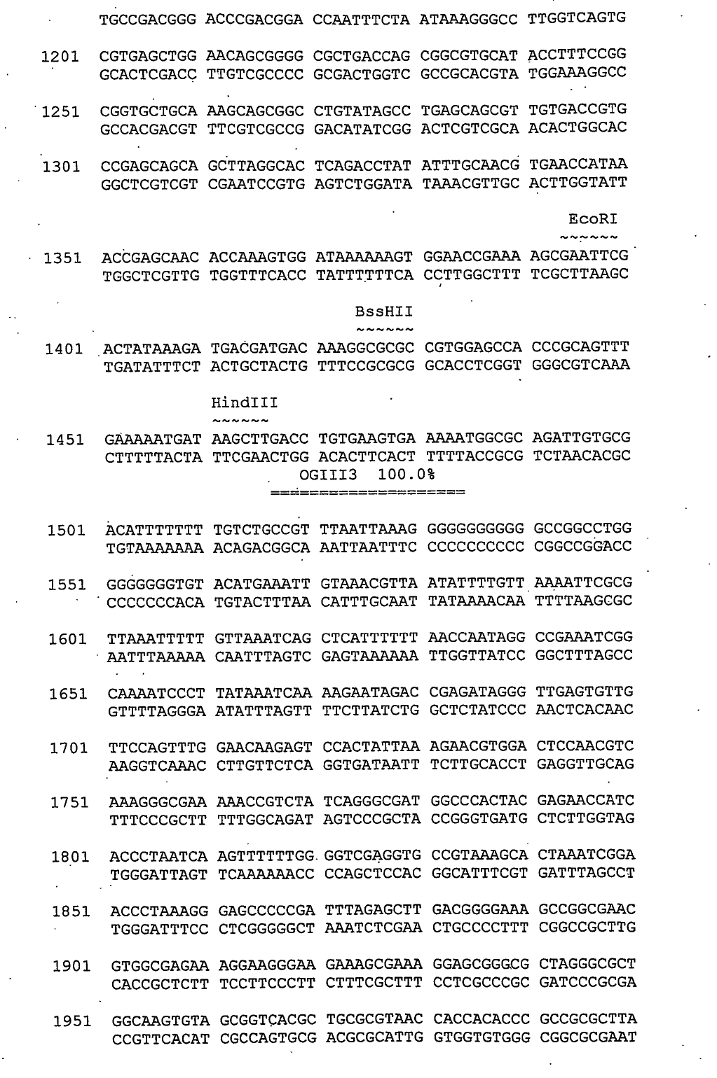

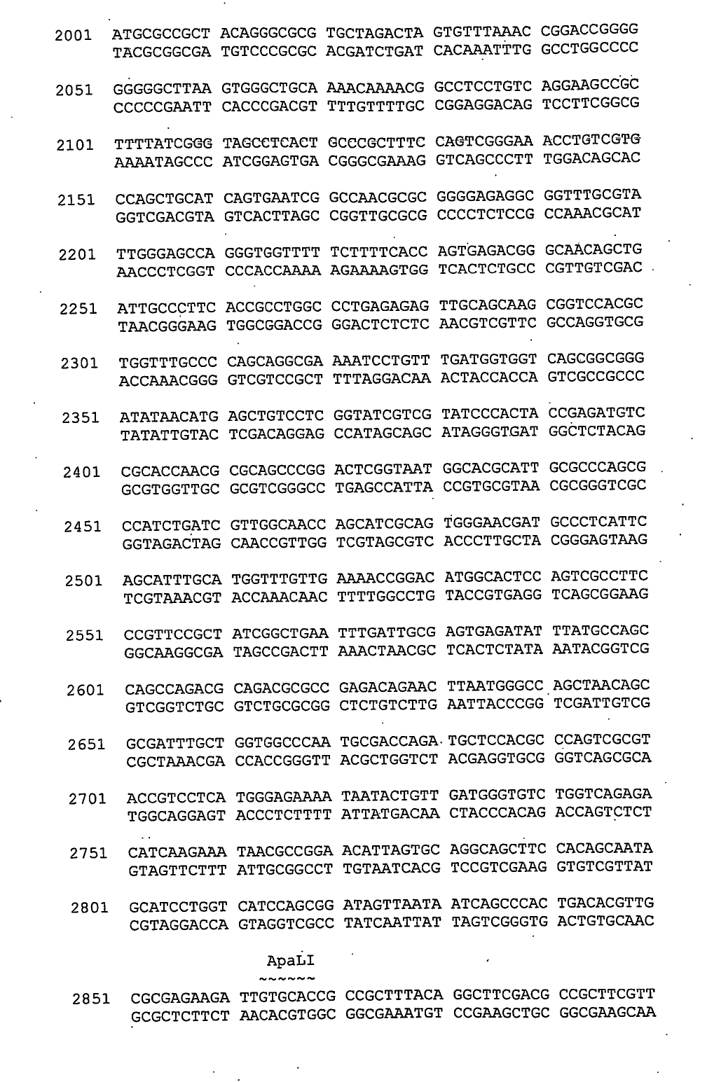

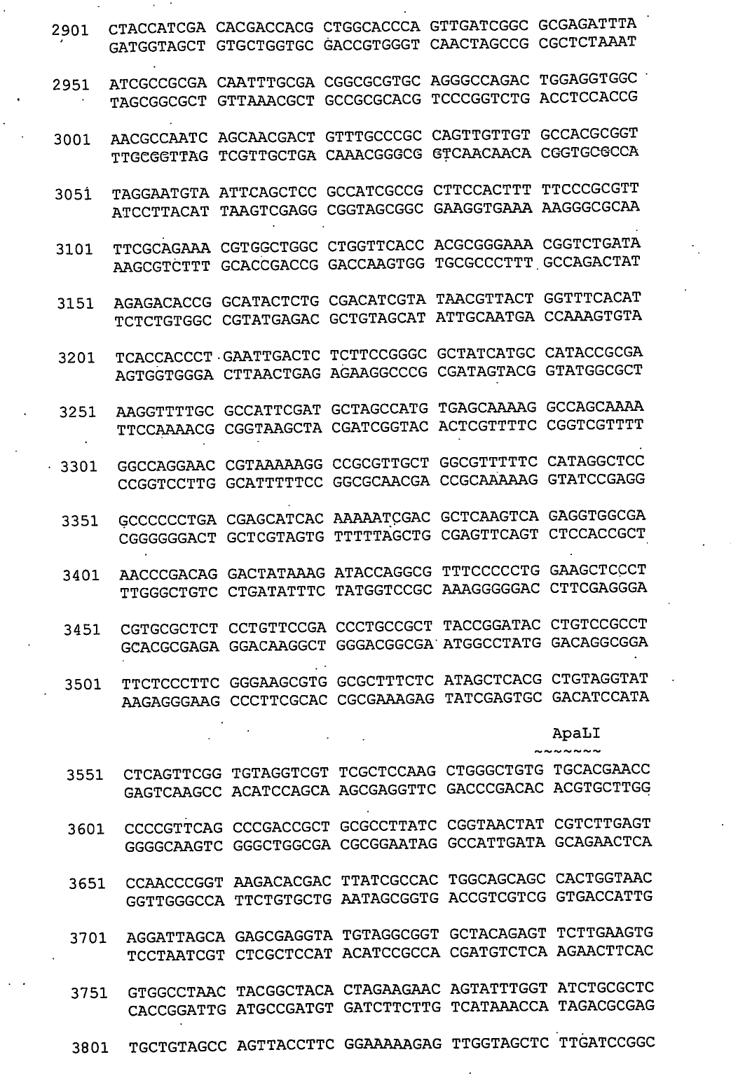

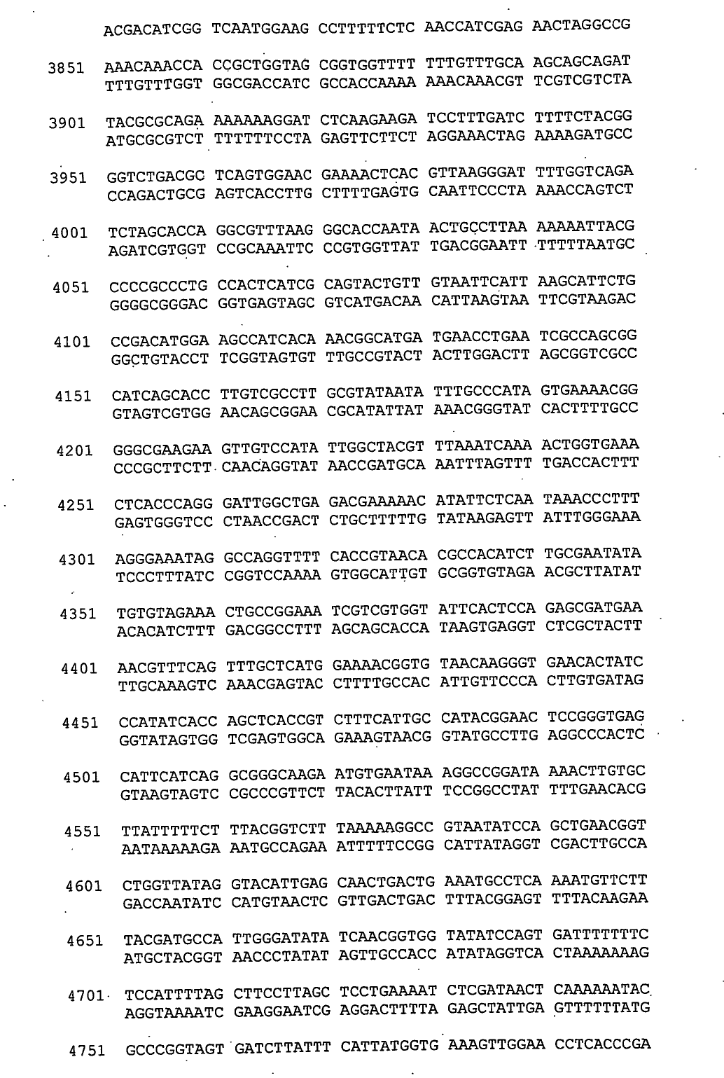

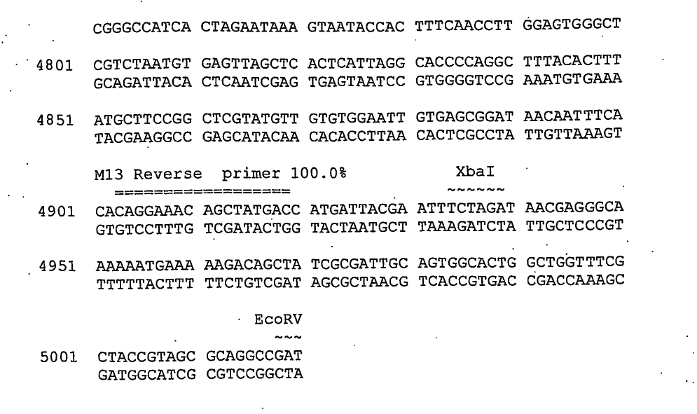

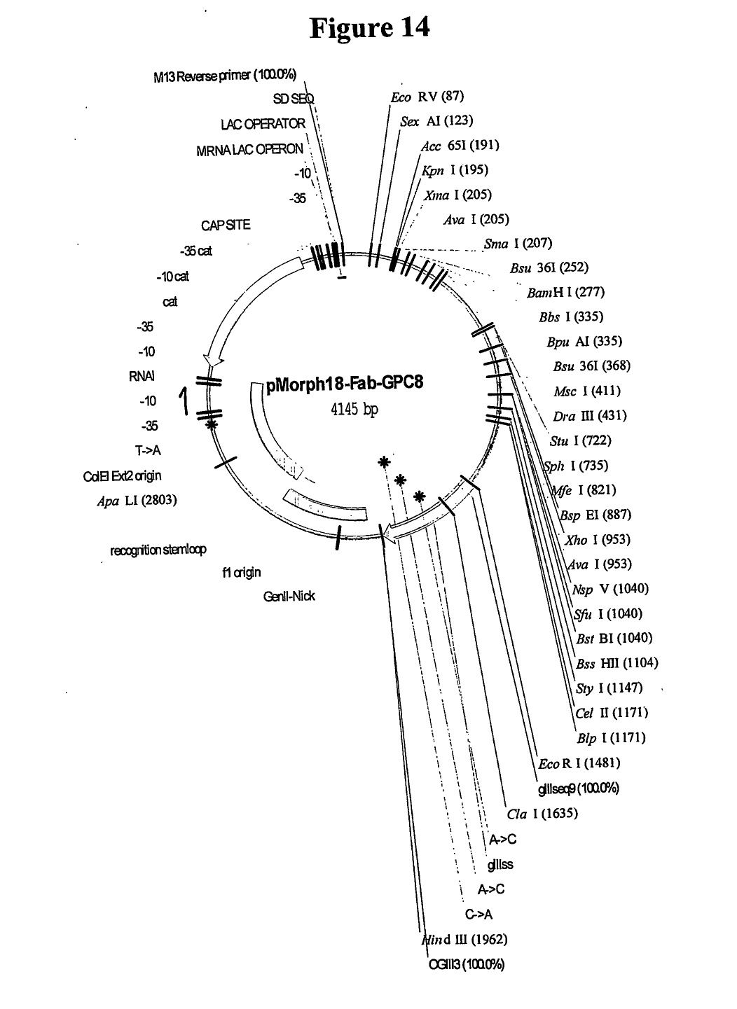

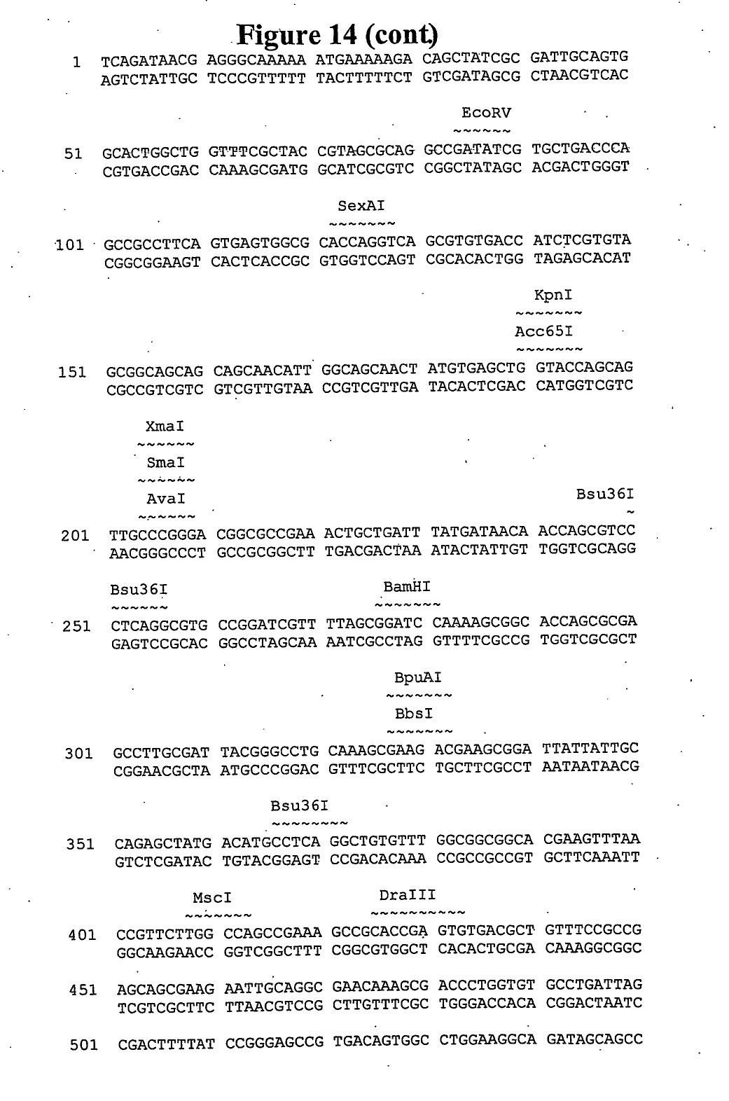

For example, the Fab fragment MS-GPC-8-Fab/B8-Fab (see 3.1) was cloned via XbaI/EcoRI from pMx9-Fab_GPC-8 into pMORPH18_Fab, a phagemid-based vector for phage display of Fab fragments, to generate pMORPH18_Fab_GPC-8 (see FIG. 14). A lambda chain pool comprising a unique DraIII restriction endonuclease site (Knappik et al., 2000) was cloned into pMORPH18_Fab_GPC-8 cut with NsiI and DraIII (see vector map of pMORPH8_Fab_GPC-8 in FIG. 14).

The resulting Fab optimization library was screened by two rounds of panning against MHC-class II DRA*0101/DRB1*0401 (prepared as above) as described in 2.3 with the exception that in the second round the antigen concentration for coating was decreased to 12 ng/wen. FACS identified optimized clones as described above in 2.5.

Finally, 12 Fabs with improved K_{off} values were selected from the B8, B8M and 8A libraries. The best clone identified (MS-GPC-8-17/7BA) had a K_{d} of about 58 nM, corresponding to a 5-fold affinity improvement compared to the best unoptimized clone MS-GPC-8/B8 (Table 3e). Libraries 17, E6 and 8A did not yield many clones with improved K_{off} values. Binders selected from the B8 library showed different L-CDR3-sequences, but all maintained the parental H-CDR2-sequence (Knappik et al., 2000), suggesting that the latter is critical for antibody-antigen interaction. For further affinity-improvement, we focussed on binders from the B8 and B8M library.

Seven of these clones, MS-GPC-8-1, MS-GPC-8-6, MS-GPC-8-9, MS-GPC-8-10, MS-GPC-8-17/7BA, MS-GPC-8-18 and MS-GPC-8-27, were further characterized and showed cell killing activity as found for the starting fragment MS-GPC-8/B8. Table 1 contains the sequence characteristics of MS-GPC-8-1, MS-GPC-8-6, MS-GPC-8-9, MS-GPC-8-10, MS-GPC-8-17/7BA, MS-GPC-8-18 and MS-GPC-8-27. The VH and VL families and the CDR3s listed refer to the HuCAL consensus-based antibody genes as described (Knappik et al., 2000). The full sequences of the VH and VL domains of MS-GPC-8-6, MS-GPC-8-10, MS-GPC-8-17/7BA and MS-GPC-8-27 are shown in FIG. 15.

The optimized Fab forms of the anti-HLA-DR antibody fragments MS-GPC-8-6 and MS-GPC-8-17 showed improved characteristics over the starting MS-GPC-8/B8. For example, the EC₅₀ of the optimized antibodies was 15-20 and 5-20 nM (compared to 20-40 nM for MS-GPC-8/B8, where the concentration is given as the concentration of the bivalent cross-linked Fab dimer), and the maximum capacity to kill MHH-Call 4 cells determined as 76 and 78% for MS-GPC-8-6 and MS-GPC-8-17 (compared to 65% for MS-GPC-8) respectively.

In the second round, L-CDR1-optimization is performed. The L-CDR1 library was generated from a pool of the 20 best Fab clones, of which 16 (including 7BA) derived from the L-CDR3 optimization and 4 from the H-CDR3 optimization. To force off-rate selection, prolonged wash cycles and competing antigen were applied to the pool-library.

Specifically, the VL CDR1 regions of a set of anti-HLA-DR antibody fragments derived from MS-GPC-8/B8 (including MS-GPC-8-10 and MS-GPC-8-27) were optimized by cassette mutagenesis using trinucleotide-directed mutagenesis (Virnekäs et al., 1994). In brief, a Vλ1 CDR1 library cassette was synthesized containing six randomized positions (total variability: 7.43×10⁶), and was cloned into a Vλ1 framework.

The CDR1 library was digested with EcoRV and BbsI, and the fragment comprising the CDR1 library ligated into the lambda light chains of the MS-GPC-8-derived Fab antibody fragments in pMORPH18_Fab (as described above), digested with EcoRV and BbsI. The resulting library was screened as described above.

The pool-library was subjected to two rounds of standard manual solid-phase panning using decreasing amounts of antigen (250 ng and 7.5 ng/well purified protein) or increasing concentrations of NH₄SCN (100 mM, 500 mM and 2500 mM), using either 2-fold serial dilutions of purified HLA-protein between 250 ng and 7.5 ng/well, or alternatively, constant amounts of 250 ng/well of protein in combination with an additional 30 min incubation step of different amounts of ammonium-isothiocyanate (100 mM, 500 mM and 2500 mM) between the standard wash-protocol and the elution step. In order to enforce off-rate-selection an additional manual solid-phase-panning of 0.3 selection rounds was performed with the pool-library using 250 ng/well of coated HLA-protein in combination with longer washes (starting from 6×30 min in the first up to 24×30 min in the 3^{rd} panning-round) and including different amounts of competing antigen (from 20 nM up to 500 nM) in be wash-buffer.

This strategy yielded Fabs with affinities of ˜3 nM (Table 3e). Ten clones were identified as above by binding specifically to HLA-DR (MS-GPC-8-6-2, MS-GPC-8-6-19, MS-GPC-8-6-27, MS-GPC-8-6-45, MS-GPC-8-6-13/305D3, MS-GPC-8-647, MS-GPC-8-10-57/1C7277, MS-GPC-8-27-7, MS-GPC-8-27-10 & MS-GPC-8-27-41/1D09C3) and showed cell killing activity as found for the starting fragments MS-GPC-8, MS-GPC-8-10 and MS-GPC-8-27. Table 1 contains the sequence characteristics of MS-GPC-8-6-2, MS-GPC-8-6-19, MS-GPC-8-6-27, MS-GPC-8-645, MS-GPC-8-6-13, MS-GPC-8-6-47, MS-GPC-8-10-57, MS-GPC-8-27-7, MS-GPC-8-27-10 & MS-GPC-8-27-41. The VH and VL families and the CDR3s listed refer to the HuCAL consensus-based antibody genes as described (Knappik et al., 2000), the full sequences of the VH and VL domains of MS-GPC-8-6-13, MS-GPC-8-10-57 and MS-GPC-8-27-41 are shown in FIG. 15.

From these 10 clones, four Fab fragments were chosen (MS-GPC-8-6-2, MS-GPC-8-6-13/305D3, MS-GPC-8-10-57/1C7277 and MS-GPC-8-27-41/1D09C3) as demonstrating significantly improved EC₅₀ of cell killing as described in example 10. Table 1 shows the sequences of clones optimised at the CDR1 region.

Optimisation procedures not only increased the biological efficacy of anti-HLA-DR antibody fragments generated by the optimisation process, but a physical characteristic—affinity of the antibody fragment to HLA-DR protein—was also substantially improved. For example, the affinity of Fab forms of MS-GPC-8/B8 and its optimised descendents was measured using a surface plasmon resonance instrument (Biacore, Upsala Sweden) according to example 7. The affinity of the MS-GPC-8/B8 parental Fab was improved over 100 fold from 346 nM to ˜60 nM after VL CDR3 optimisation and further improved to single digit nanomolar affinity (range 3-9 nM) after VL CDR3+1 optimisation (Table 2).

5. Generation of IgG

5.1 Construction of HuCAL-Immunoglobulin Expression Vectors

Three Fabs (305D3, 1D09C3, and 1C7277) obtained above were converted into IgG4 format, expressed and purified for affinity determination (see below). All 3 IgG₄ mAbs exhibited sub-nanomolar affinities (0.3-0.6 nM; Table 3e), and retained their specificity (FIG. 2).

Heavy chains were cloned as follows. The multiple cloning site of pcDNA3.1+(Invitrogen) was removed (NheI/ApaI), and a stuffer compatible with the restriction sites used for HuCAL-design was inserted for the ligation of the leader sequences (NheI/EcoRI), VH-domains (EcoRI/BlpI, with EcoRI being compatible with the restriction site MfeI present at the 5′-end of the VH domains) and the immunoglobulin constant regions (BlpI/ApaI). The leader sequence (EMBL M83133) was equipped with a Kozak sequence (Kozak, 1987). The constant regions of human IgG₁ (PIR J00228), IgG₄ (EMBL K01316) and serum IgA₁ (EMBL J00220) were dissected into overlapping oligonucleotides with lengths of about 70 bases. Silent mutations were introduced to remove restriction sites non-compatible with the HuCAL-design. The oligonucleotides were spliced by overlap extension-PCR. By cloning the VH domain polynucleotide sequences digested with MfeI into the pcDNA3.1+-derived vector digested with EcoRI, the first three codons of the VH domain polynucleotide sequences are changed to “CAG GTG GAA”, thus changing the first three amino acid residues to “QVE”.

Light chains were cloned as follows. The multiple cloning site of pcDNA3.1/Zeo+(Invitrogen) was replaced by two different stuffers. The κ-stuffer provided restriction sites for insertion of a κ-leader (NheI/EcoRV), HuCAL-scFv Vκ-domains (EcoRV/BsiWI) and the κ-chain constant region (BsiWI/ApaI). The corresponding restriction sites in the λ-stuffer were NheI/EcoRV (λ-leader), EcoRV/HpaI (Vλ-domains) and HpaI/ApaI (λ-chain constant region). The κ-leader (EMBL Z00022) as well as the λ-leader (EMBL L27692) were both equipped with Kozak sequences. The constant regions of the human κ-(EMBL J00241) and λ-chain (EMBL M18645) were assembled by overlap extension-PCR as described above.

5.2 Generation of IgG-Expressing CHO-Cells

All cells were maintained at 37° C. in a humidified atmosphere with 5% CO₂ in media recommended by the supplier. CHO-K1 (CRL-9618) were from ATCC and were co-transfected with an equimolar mixture of IgG heavy and light chain expression vectors. Double-resistant transfectants were selected with 600 μg/ml G₄₁₈ and 300 μg/ml Zeocin (Invitrogen) followed by limiting dilution. The supernatant of single clones was assessed for IgG expression by capture-ELISA. Positive clones were expanded in RPMI-1640 medium supplemented with 10% ultra-low IgG-FCS (Life Technologies). After adjusting the pH of the supernatant to 8.0 and sterile filtration, the solution was subjected to standard protein A column chromatography (Poros 20A, PE Biosystems).

The IgG forms of anti-HLA-DR antigen binding domains show improved characteristics over the antibody fragments. These improved characteristics include affinity (Example 7) and killing efficiency (Examples 9, 10 and 14).

6. HLA-DR Specificity Assay and Epitope Mapping

To demonstrate that antigen-binding domains selected from the HuCAL library bound specifically to a binding site on the N-terminal domain of human MHCII receptor largely conserved between alleles and hitherto unknown in the context of cell killing by receptor cross linking, we undertook an assessment of their binding specificity, and it was attempted to characterise the binding epitope.

The Fab antibody fragments MS-GPC-8-27-7, MS-GPC-8-27-10, MS-GPC-8-6-13, MS-GPC-8-27-41/1D09C3, MS-GPC-8-647, MS-GPC-8-10-57/1C7277, MS-GPC-8-6-27, MS-GPC-8/B8 and MS-GPC-8-6 showed specificity of binding to HLA-DR protein but not to non-HLA-DR proteins. Fab fragments selected from the HuCAL library were tested for reactivity with the following antigens: HLA-DR protein (DRA*0101/DRB1*0401; prepared as example 1, and a set of unrelated non-HLA-DR proteins consisting of BSA, testosterone-BSA, lysozyme and human apotransferrin. An empty well (Plastic) was used as negative control. Coating of the antigen MHCII was performed over night at 1 μg/well in PBS (Nunc-MaxiSorp™) whereas for the other antigens (BSA, Testosterone-BSA, Lysozyme, Apotransferrin) 10 μg/well was used. Next day wells were blocked in 5% non-fat milk for 1 hr followed by incubation of the respective antibodies (anti-MHCII-Fabs and an unrelated Fab (Mac1-8A)) at 100 ng/well for 1 hour. After washing in PBS the anti-human IgG F(ab′)₂-peroxidase-conjugate at a 1:10,000 dilution in TBS (supplemented with 5% w/v non-fat dry-milk/0.05% v/v Tween 20) was added to each well for 1 h. Final washes were carried out in PBS followed the addition the substrate POD (Roche); Color-development was read at 370 nM in an ELISA-Reader.

All anti-HLA-DR antibody fragments MS-GPC-8-27-7, MS-GPC-8-27-10, MS-GPC-8-6-13, MS-GPC-8-27-41, MS-GPC-8-647, MS-GPC-8-10-57, MS-GPC-8-6-27, MS-GPC-8 and MS-GPC-8-6 demonstrated high specificity for HLA-DR, as evidenced by the much higher mean fluorescence intensity resulting from incubation of these antibody fragments with HLA-DR derived antigens compared to controls (FIG. 1a). In a similar experiment, the Fab fragments MS-GPC-1, MS-GPC-6, MS-GPC-8 and MS-GPC-10 were found to bind to both the DRA*0101/DRB1*0401 (prepared as above) as well as to a chimeric DR-IE consisting of the N-terminal domains of DRA*0101 and DRB1*0401 with the remaining molecule derived from a murine class II homologue IEd (Ito et al., 1996) (FIG. 1b).

To demonstrate the broad-DR reactivity of anti-HLA-DR antibody fragments and IgGs of the invention, the scFv forms of MS-GPC-1, 6, 8 and 10, and IgG forms of MS-GPC-8, MS-GPC-8-10-57, MS-GPC-8-27-51 & MS-GPC-8-6-13 were tested for reactivity against a panel of Epstein-Barr virus transformed B cell lines obtained from ECACC (Salisbury UK), each homozygous for one of the most frequent DR alleles in human populations (list of cell lines and alleles shown in FIG. 2). The antibody fragments were also tested for reactivity against a series of L cells transfected to express human class II isotypes other than DRB1: L105.1, L257.6, L25.4, L256.12 & L21.3 that express the molecules DRB3*0101, DRB4*0101, DP0103/0402, DP 0202/0201, and DQ0201/0602 respectively (Klohe et al., 1988).

Reactivity of an antigen-binding fragment to the panel of cell-lines expressing various MHC-class II molecules was demonstrated using an immunofluorescence procedure as for example, described by Otten et al (1997). Staining was performed on 2×10⁵ cells using an anti-FLAG M2 antibody as the second reagent against the M2 tag carried by each anti-HLA-DR antibody fragment and a fluorescein labelled goat anti-mouse Ig (BD Pharmingen, Torrey Pine, Calif., USA) as a staining reagent. Cells were incubated at 4° C. for 60 min with a concentration of 200 nM of the anti-HLA-DR antibody fragment, followed by the second and third-antibody at concentrations determined by the manufacturers. For the IgG form, the second antibody was omitted and the IgG detected using a FITC-labeled mouse anti-human IgG₄ (Serotec, Oxford, UK). Cells were washed between incubation steps. Finally the cells were washed and subjected to analysis using a FACS Calibur (BD Immunocytometry Systems, San Jose, Calif., USA).

FIG. 2 shows that the scFv-fragments MS-GPC-1, 6, 8 and 10, and IgG forms of MS-GPC-8, MS-GPC-8-10-57, MS-GPC-8-27-51 & MS-GPC-8-6-13 react with all DRB1 allotypes tested. This observation taken together with the observation that all anti-HLA-DR antibody fragments react with chimeric DR-IE, suggests that all selected anti-HLA-DR antibody fragments recognize the extracellular first domain of the monomorphic DRα chain or a monomorphic epitope on extracellular first domain of the DRβ chain.

We then attempted to localize the binding domains of MS-GPC-8-10-57 and MS-GPC-8-27-41 further by examining competitive binding with murine antibodies for which the binding domains on HLA-DR are known. The murine antibodies L243 and LB3.1 are known to bind to the β1 domain, 1-1C4 and 8D1 to the β1 domain and 10F12 to the β2 domain (Vidovic et al. 1995b). To this end, an assay was developed wherein a DR-expressing cell line (LG-2) was at first incubated with the IgG₄ forms of MS-GPC-8-10-57 or MS-GPC-8-27-41, the Fab form of MS-GPC-8-10-57 or the Fab form of GPC 8, and an unrelated control antibody. Subsequently murine antibodies were added and the murine antibodies were detected. If the binding site of MS-GPC-8-10-57 or MS-GPC-8-27-41 overlaps with the binding of a murine antibody, then a reduced detection of the murine antibody is expected.

Binding of the IgG₄ forms of GPC-8-27-41 and MS-GPC-8-10-57 and the Fab form of MS-GPC-8-10-57 substantially inhibited (mean fluorescence intensity reduced by >90%) the binding of 1-1C4 and LD1, whereas L243, LB3.1 and 10F12 and a control were only marginally affected. The Fab form of MS-GPC-8 reduced binding of 1-1C4 by ˜50% (mean fluorescence dropped from 244 to 118), abolished 8D1 binding and only marginally affected binding of L243, LB3.1 and 10F12 or the control. An unrelated control antibody had no effect on either binding. Thus, MS-GPC-8-10-57 and MS-GPC-8-27-41 seem to recognise a β1 domain epitope that is highly conserved among allelic HLA-DR molecules.

The whole staining procedure was performed on ice. 1×10⁷ cells of the human B-lymphoblastoid cell line LG-2 was preblocked for 20 rain. in PBS containing 2% FCS and 35 μg/ml Guinea Pig IgG (“FACS-Buffer”). These cells were divided into 3 equal parts A, B, and C of approximately 3.3×10⁶ cells each, and it was added to A) 35 μg MS-GPC-8-10-57 or MS-GPC-8-27-41 IgG₄, to B) 35 μg MS-GPC-8-10-57 Fab or MS-GPC-8 Fab, and to C) 35 μg of an unrelated IgG₄ antibody as negative control, respectively, and incubated for 90 min. Subsequently A, B, C were divided in 6 equal parts each containing 5.5×10⁵ cells, and 2 μg of the following murine antibodies were added each to one vial and incubated for 30 min: 1) purified mIgG; 2) L243; 3) LB3.1; 4) 1-1 C4; 5) 8D1; 6) 10F12. Subsequently, 4 ml of PBS were added to each vial, the vials were centrifuged at 300×g for 8 min, and the cell pellet resuspended in 50 μl FACS buffer containing a 1 to 25 dilution of a goat-anti-murine Ig-FITC conjugate at 20 μg/ml final concentration (BD Pharmingen, Torrey Pines, Calif., USA). Cells were incubated light-protected for 30 min. Afterwards, cells were washed with 4 ml PBS, centrifuged as above and resuspended in 500 μl PBS for analysis in the flow cytometer (FACS Calibur, BD Immunocytometry Systems, San Jose, Calif., USA).

The PepSpot technique (U.S. Pat. No. 6,040,423; Heiskanen et al., 1999) is used to further identify the binding epitope for MS-GPC 8-10-57. Briefly, an array of 73 overlapping 15-mer peptides is synthesised on a cellulose membrane by a solid phase peptide synthesis spotting method (WO 00/12575). These peptide sequences are derived from the sequence of the α1 and β1 domains of HLA-DR4Dw14, HLA-DRA1*0101 (residues 1-81) and HLA-DRB1*0401 (residues 2-92), respectively, and overlap by two amino acids. Second, such an array is soaked in 0.1% Tween-20/PBS (PBS-T), blocked with 5% BSA in PBS-T for 3 hours at room temperature and subsequently washed three times with PBS-T. Third, the prepared array is incubated for 90 minutes at room temperature with 50 ml of a 5 mg/l solution of the IgG form of GPC-8-10-57 in 1% BSA/PBS-T. Fourth, after binding, the membrane is washed three times with PBS-T and subsequently incubated for 1 hour at room temperature with a goat anti-human light chain antibody conjugated to horseradish peroxidase diluted 1/5,000 in 1% BSA/PBS-T. Finally, the membrane is washed three times with PBS-T and any binding determined using chemiluminescence detection on X-ray film. As a control for unspecific binding of the goat anti-human light chain antibody, the peptide array is stripped by the following separate washings each at room temperature for 30 min: PBS-T (2 times), water, DMF, water, an aequeous solution containing 8 M urea, 1% SDS, 0.5% DTT, a solution of 50% ethanol, 10% acetic acid in water (3 times each) and, finally, methanol (2 times). The membrane is again blocked, washed, incubated with goat anti-human 1 light chain antibody conjugated to horseradish peroxidase and developed as described above.

7. Affinity of Anti-HLA-DR Antibody and Antibody Fragments

In order to demonstrate the superior binding properties of anti-HLA antibody fragments of the invention, we measured their binding affinities to the human MHC class II DR protein (DRA*0L01/DRB1*0401) using standard equipment employing plasmon resonance principles. Surprisingly, we achieved affinities in the sub-nanomolar range for IgG forms of certain anti-HLA-DR antibody fragments of the invention. For example, the affinity of the IgG forms of MS-GPC-8-27-41, MS-GPC-8-6-13 & MS-GPC-8-10-57 was measured as 0.3, 0.5 and 0.6 nM respectively (Table 3a). Also, we observed high affinities in the range of 2-8 nM for Fab fragments affinity matured at the CDR1 and CDR3 light chain regions (Table 3b). Fab fragments affinity matured at only the CDR3 light chain region showed affinities in the range of 40 to 100 nM (Table 3c), and even Fab fragments of non-optimised HuCAL antigen binding domains showed affinities in the sub μM range (Table 3d). Only a moderate increase in Kₒₙ (2-fold) was observed following CDR3 optimisation (Kₒₙ remained approximately constant throughout the antibody optimization process in the order of 1×10⁵ M⁻¹s⁻¹), whilst a significant decrease in K_{off} was a surprising feature of the optimisation process—sub 100 s⁻¹, sub 10 s⁻¹, sub 1 s⁻¹ and sub 0.1 s⁻¹ for the unoptimised Fabs, CDR3 optimised Fabs, CDR3/CDR1 optimised Fabs and IgG forms of anti-HLA-DR antibody fragments of the invention.

The affinities for anti-HLA antibody fragments of the invention were measured as follows. All measurements were conducted in HBS buffer (20 mM HEPES, 150 mM NaCl, pH 7.4) at a flow rate of 20 μp/min at 25° C. on a BIAcore3000 instrument (Biacore AB, Sweden). MHC class II DR protein (prepared as example 1) was diluted in 100 mM sodium acetate pH 4.5 to a concentration of 50-100 mg/ml, and coupled to a CM5 chip (Biacore AB) using standard EDC-NHS coupling chemistry with subsequent ethanolamine treatment as manufacturers directions. The coating density of MHCII was adjusted to between 500 and 4000 RU. Affinities were measured by injection of 5 different concentrations of the different antibodies and using the standard software of the Biacore instrument. Regeneration of the coupled surface was achieved using 10 mM glycine pH 2.3 and 7.5 mM NaOH.

8. Multivalent Killing Activity of Anti HLA-DR Antibodies and Antibody Fragments

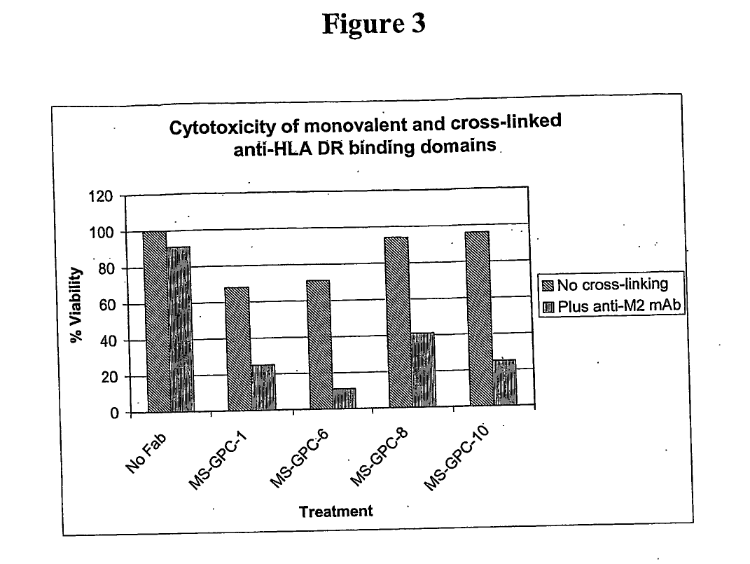

To demonstrate the effect of valency on cell killing, a cell killing assay was performed using monovalent, bivalent and multivalent compositions of anti-HLA-DR antibody fragments of the invention against GRANTA-519 cells. Anti-HLA-DR antibody fragments from the HuCAL library showed much higher cytotoxic activity when cross-linked to form a bivalent composition (60-90% killing at antibody fragment concentration of 200 nM) by co-incubation with anti-FLAG M2 mAb (FIG. 3) compared to the monovalent form (5-30% killing at antibody fragment concentration of 206 nM). Incubation of cell lines alone or only in the presence of anti-FLAG M2 mAb without co-incubation of anti-HLA-DR antibody fragments did not lead to cytotoxicity as measured by cell viability. Treatment of cells as above but using 50 n-M of the IgG₄ forms (naturally bivalent) of the antibody fragments MS-GPC-8, MS-GPC-8-6-13, MS-GPC-8-10-57 and MS-GPC-8-27-41 without addition of anti-FLAG M2 mAb showed a killing efficiency after 4 hour incubation of 76%, 78%, 78% and 73% respectively.

Furthermore, we observed that higher order valences of the anti-HLA-DR antibody fragments further decrease cell viability significantly. On addition of Protein G to the incubation mix containing the IgG form of the anti-HLA-DR antibody fragments, the multivalent complexes thus formed further decrease cell viability compared to the bivalent composition formed from incubation of the anti-HLA-DR antibody fragments with only the bivalent IgG form.

The killing efficiency of anti-HLA-DR antibody fragments selected from the HuCAL library was tested on the HLA-DR positive tumor cell line GRANTA-519 (DSMZ, Germany). 2×10⁵ cells were incubated for 4 h at 37° C. under 6% CO₂ with 200 nM anti-HLA-DR antibody fragments in RPMI 1640 (PAA, Germany) supplemented with 2.5% heat inactivated FBS (Biowhittaker Europe, BE), 2 mM L-glutamine, 1% non-essential amino acids, 1 mM sodium pyruvate and 0.1 mg/ml kanamycin. Each anti-HLA-DR antibody fragment was tested for its ability to kill activated tumor cells as a monovalent anti-HLA-DR antibody fragment or as a bivalent composition by the addition of 100 nM of a bivalent cross-linking anti-FLAG M2 mAb. After 4 h incubation at 37° C. under 6% CO₂, cell viability was determined by trypan blue staining and subsequent counting of remaining viable cells (Current Protocols in Immunology, 1997).

The above experiment was repeated using KARPAS-422 cells against a multivalent form of IgG forms of MS-GPC-8-10-57 and MS-GPC-8-27-41 prepared by a pre-incubation with a dilution series of the bacterial protein Protein G. Protein G has a high affinity and two binding sites for IgG antibodies, effectively cross-linking them to yield a total binding valency of 4. In a control using IgG alone without preincubation with Protein G, approximately 55% of cells were killed, while cell killing using IgG pre-incubated with Protein G gave a maximum of approximately 75% at a molar ratio of IgG antibody/protein G of ˜6 (based on a molecular weight of Protein G of 28.5 kD). Higher or lower molar ratios of IgG antibody/Protein G approached the cell killing efficiency of the pure IgG antibodies.

9. Killing Efficiency of Anti-HLA-DR Antibody Fragments

Experiments to determine the killing efficiency of the anti-HLA-DR cross-linked antibody fragments against other tumor cell lines that express HLA-DR molecules were conducted analogous to example 8. Tumor cell lines that show greater than 50% cell killing with the cross linked Fab form of MS-GPC-8 after 4 h incubation include MHH-CALL4, MN 60, BJAB, BONNA-12 which represent the diseases B cell acute lymphoid leukemia, B cell acute lymphoid leukemia, Burkitt lymphoma and hairy cell leukemia respectively. Use of the cross-linked Fab form of the anti-HLA-DR antibody fragments MS-GPC-1, 6 and 10 also shows similar cytotoxic activity to the above tumor cell lines when formed as a bivalent agent using the cross-linking anti-FLAG M2 mAb.

The method described in example 8 was used to determine the maximum killing capacity for each of the cross-linked bivalent anti-HLA-DR antibody fragments against PRIESS cells. The maximum killing capacity observed for MS-GPC-1, MS-GPC-6, MS-GPC-8 & MS-GPC-10 was measured as 83%, 88%, 84% and 88% respectively. Antibody fragments generated according to example 4, when cross linked using anti-FLAG M2 mAb as above, also showed improved killing ability against GRANTA and PRIESS cells (Table 4).

10. Killing Efficiency of Human Anti-HLA-DR IgG Antibodies

The optimized IgG₄ mAbs were tested for induction of tumor cell death on a panel of 24 DR+ and 4 DR: cell lines, representing a variety of lymphoma/leukemia types (Table 5). Compared to corresponding murine antibodies (Vidovic et al, 1995b; Nagy & Vidovic, 1996; Vidovic & Toral; 1998), we were surprised to observe significantly improved killing efficiency of IgG forms of certain anti-HLA-DR antibody fragments of the invention (Table 5). The killing is dependent on HLA-DR expression, but is HLS-DR subtype independent.

For the cell killing assay, cells at 2×10⁶/ml concentration were incubated in RPMI 1640 supplemented with 2.5% fetal calf serum (Biowhittaker Europe, Belgium) and different concentrations (50 nM in most experiments) of human anti-DR mAb at 37° C. for 4 hrs (and 24 h in some experiments). Control cultures were without mAb or with a murine anti-DR mAb 10F12 that fails to induce cell death. Cell cultures were set up in duplicate in flat bottom 96 well plates. Since dead cells disintegrate very fast (within 30 min),% killing was determined based on viable cell recovery as follows: (viable untreated—viable treated/viable untreated)×100. Viable and dead cells were distinguished by trypan blue staining for light microscopy, fluorescein diacetate (FDA; 100 μg/ml final concentration; live cells) and propidium iodide (PI, 40 μg/ml final concentration; dead cells) for fluorescent microscopy, and PI for FACS analysis. To obtain absolute cell counts by FACS analysis, each culture was supplemented with equal amounts of FACS “Truecount” calibrating beads. Cell counts were determined by the formula: viable cells×total beads/counted beads. The three different methods of cell counting (light and fluorescent microscopy and FACS) yielded comparable results.

Following the method described in examples 8 and 9 and above but at 50 nM, repeated measurements (3 to 5 replica experiments where cell number was counted in duplicate for each experiment) were made of the killing efficiency of the IgG forms of certain antibody fragments of the invention.

The mAbs induced death in a wide range (23 of the 25) DR+lymphoid tumor lines. When applied at a final concentration of only 50 nM, IgGs of the antibody fragments MS-GPC-8/B8, MS-GPC-8-6-13/305D3, MS-GPC-8-10-57/1C7277 & MS-GPC-8-27-41/1D09C3 killed more than 50% of cells from 17, 20, 19 and 22 respectively of a panel of 25 human tumor cell lines that express HLA-DR antigen at a level greater than 10 fluorescent units as determined by example 11. For comparison, two murine anti-DR mAbs, L243 (Vidovic et al, 1995b) and 8D1 (Vidovic & Toral; 1998) known to induce cell death^{7,10} were tested on the same panel at 4 fold higher concentration (200 nM) than the human mAbs. The murine mAbs usually killed less cells than human mAbs, or failed to induce death in some DR⁺ lines. Over all, they reduced cell viability to a level below 50% viable cells in only 13 and 12 of the 25 HLA-DR expressing cells lines, respectively.

In direct comparisons, the human mAbs achieved 50% killing efficiency at 20 to 30 fold lower concentrations than the murine mAbs (see below). Statistical analysis of the data in Table 5 revealed a non-linear correlation between killing efficiency and the level of DR expression, with a significantly greater killing efficiency and better correlation for the human mAbs because of the failure of the murine mAbs to kill a number of DR⁺ lines.

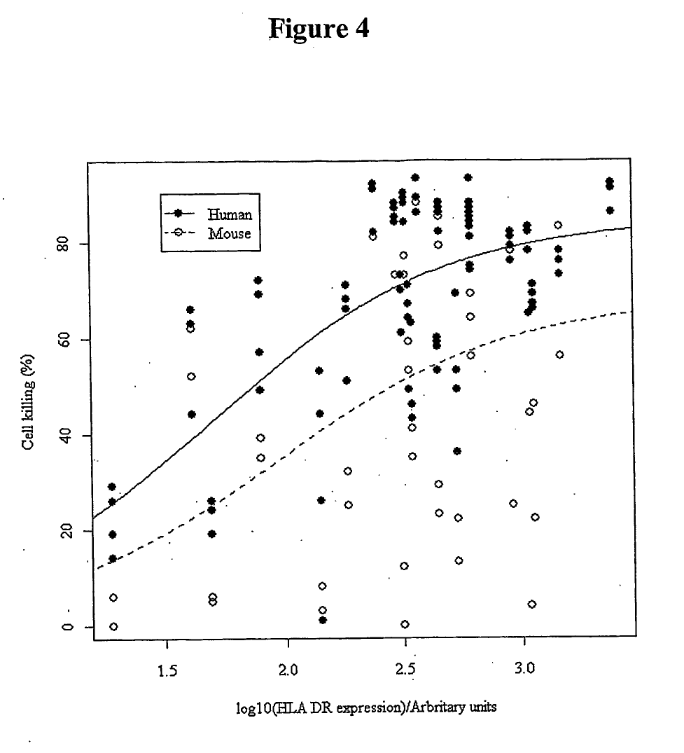

Indeed, even at the significantly increased concentration, the two murine antibodies treated at 200 nM showed significantly less efficient killing compared to the IgG forms of anti-HLA-DR antibody fragments of the invention. Not only do IgG forms of the human anti-HLA-DR antibody fragments of the invention show an overall increase in cell killing at lower concentrations compared to the murine antibodies, but they show less variance in killing efficiency across different cell lines. The coefficient of variance in killing for the human antibodies in this example is 32% (mean % killing=68+/−22% (SD)), compared to over 62% (mean % killing=49+/−31% (SD)) for the mouse antibodies. Statistically controlling for the effect on killing efficiency due to HLA expression by fitting logistic regression models to mean percentage killing against log(mean HLA-DR expression) supports this observation (FIG. 4). Not only is the fitted curve for the murine antibodies constantly lower than that for the human, but a larger variance in, residuals from the murine antibody data (SD=28%) is seen compared to the variance in residuals from the human antibody data (16%). The superior performance of human mAbs could be explained, at least in part, by their higher affinity (K_{d}-s 0.3-0.6 nM, see Table 3e, compared to L243 10 nM, and 8D1>30 nM (Z. A. Nagy, unpublished)).

The cell line MHH-PREB-1 was singled out and not accounted as part of the panel of 25 cell lines despite its expression of HLA-DR antigen at a level greater than 10 fluorescent units due to the inability of any of the above antibodies to induce any significant reduction of cell viability. This is further explained in example 12.

The viability of DR7 cell lines was not significantly affected.

11. Killing Selectivity of Antigen-Binding Domains Against a Human Antigen for Activated Versus Non-Activated Cells

Since MHC-II molecules are constitutively expressed on B lymphocytes, the most obvious potential side effect of anti-DR mAb treatment would be the killing of normal B cells. Human peripheral B cells were therefore used to demonstrate that human anti-HLA-DR mAb-mediated cell killing is dependent on cell-activation. 50 ml of heparinised venous blood was taken from an HLA-DR typed healthy donor and fresh peripheral blood mononuclear cells (PBMC) were isolated by Ficoll-Hypaque Gradient Centrifugation (Histopaque-1077; Sigma) as described in Current Protocols in Immunology (John Wiley & Sons, Inc.; 1999). Purified B cells (˜5% of peripheral blood leukocytes) were obtained from around 5×10⁷ PBMC using the B-cell isolation kit and MACS LS⁺/VS⁺ columns (Miltenyi Biotec, Germany) according to manufacturers guidelines. Successful depletion of non-B cells was verified by FACS analysis of an aliquot of isolated B cells (HLA-DR positive and CD19 positive). Double staining and analysis is done with commercially available antibodies (BD Immunocytometry Systems, San Jose, Calif., USA) using standard procedures as for example described in Current Protocols in Immunology (John Wiley & Sons, Inc.; 1999). An aliquot of the isolated B cells was tested for the ability of the cells to be activated by stimulation with Pokeweed mitogen (PWM) (Gibco BRL, Cat. No. 15360-019) diluted 1:25 in RPMI 1640 (PAA, Germany) supplemented with 10% FCS (Biowhittaker Europe, BE), 2 mM L-glutamine, 1% non-essential amino acids, 1 mM sodium pyruvate and 0.1 mg/ml kanamycin by incubation at 37° C. under 6% CO₂ for three days. Successful activation was verified by FACS analysis of HLA-DR expression on the cell surface (Current Protocols in Immunology, John Wiley & Sons, Inc.; 1999).

The selectivity for killing of activated cells versus non-activated cells was demonstrated by incubating 1×10⁶/ml B cells activated as above compared to non-activated cells, respectively with 50 nM of the IgG forms of MS-GPC-8-10-57, MS-GPC-8-27-41 or the murine IgG 10F12 (Vidovic et al., 1995b) in the medium described above but supplemented with 2.5% heat inactivated FCS instead of 10%, or with medium alone. After incubation at 37° C. under 6% CO₂ for 1 or 4 h, cell viability was determined by fluorescein diacetate staining (FDA) of viable and propidium iodide staining (PI) of dead cells and subsequent counting of the green (FDA) and red (PI) fluorescent cells using a fluorescence microscope (Leica, Germany) using standard procedures (Current Protocols in Immunology, 1997).

B cell activation was shown to be necessary for cell killing. In non-activated cells after 1 hr of incubation with the anti-HLA-DR antibodies, the number of viable cells in the media corresponded to 81%, 117% 126% and 96% of the pre-incubation cell density for MS-GPC-8-10-57 (IgG), MS-GPC-8-27-41 (IgG), 10F12 and medium alone, respectively. In contrast, the number of viable activated B cells after 1 h incubation corresponded to 23%, 42% 83% and 66% of the pre-incubation cell density for MS-GPC-8-10-57 (IgG), MS-GPC-8-27-41 (IgG), 10F12 and medium alone, respectively. After 4 hr of incubation, 78%, 83% 95% and 97% of the pre-incubation cell density for MS-GPC-8-10-57 (IgG), MS-GPC-8-27-41 (IgG), 10F12 and medium alone were found viable in non-activated cells, whereas the cell density had dropped to 23%, 24% 53% and 67% of the pre-incubation cell density for MS-GPC-8-10-57 (IgG), MS-GPC-8-27-41 (IgG), 10F12 and medium alone, respectively, in activated cells.

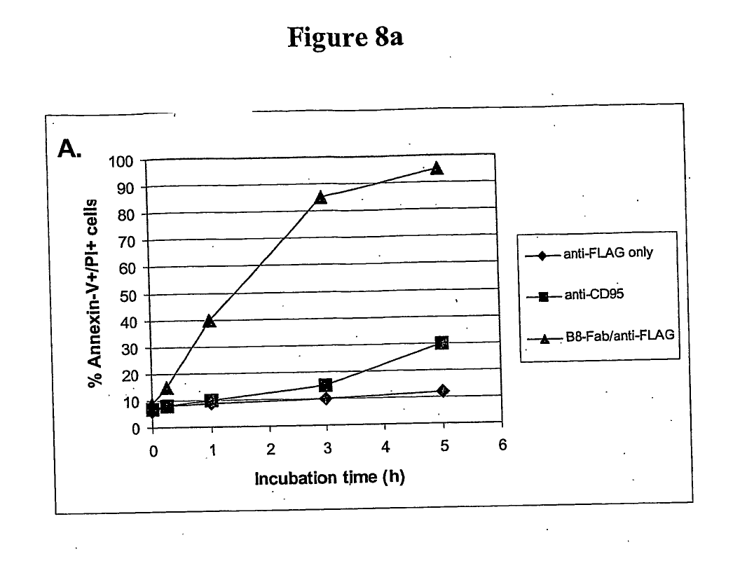

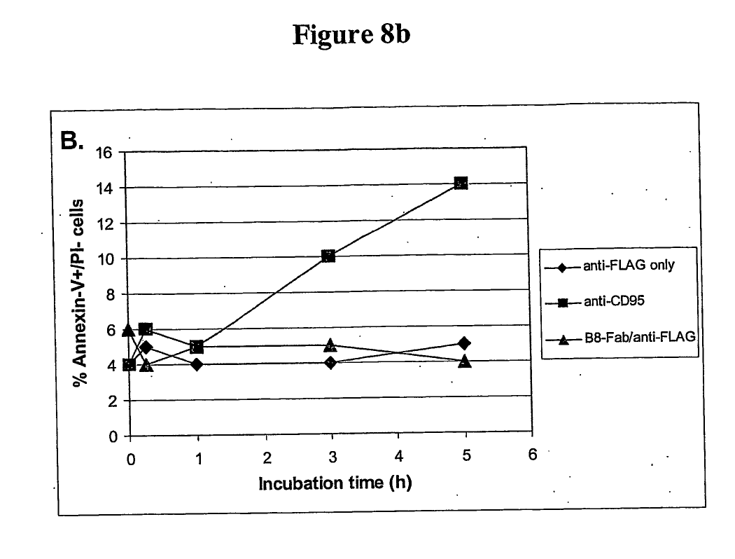

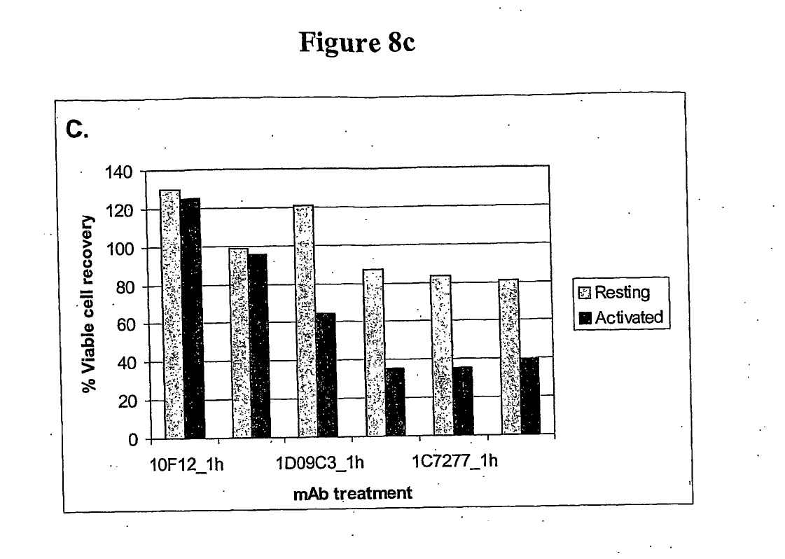

In conclusion, as shown in FIG. 8c, the viability of purified resting B cells was not significantly altered by human anti-DR mAbs. In contrast, pokeweed mitogen-activated B cells from the same donor were killed by these mAbs. No death of either unactivated or activated B cells was induced by the control antibody 10F12. Similar results were obtained with resting and lipopolysaccharide-stimulated spleenic B cells from DR-transgenic mice (Ito, K. et al. J. Exp. Med. 183:2635-2644, 1996) (data not shown). Thus, it appears that the mAbs can kill activated but not resting MHC-II positive normal cells in addition to tumor cells, suggesting a dual requirement of both MHC-II expression and cell activation for mAb-induced death. Since the majority (up to 99%) of peripheral B cells is resting, the potential side effect due to killing of normally activated B cells in a leukaemia patient is negligible.

12. Killing Activity of Anti-MLA Antibody Fragments Against the Cell Line MHH PreB 1

As evidenced in Table 5, we observed that our cross-linked anti-HLA-DR antibody fragments or IgGs did not readily kill a particular tumor cell line expressing HLA-DR at significant levels (MHH-PREB-1). We hypothesized that although established as a stable cell line, cells in this culture were not sufficiently activated. We therefore stimulated these cells with interferon-gamma, and lipopolysaccharide. Activation was evidenced by an increase in the cell surface expression of CD40 and HLA-DR.

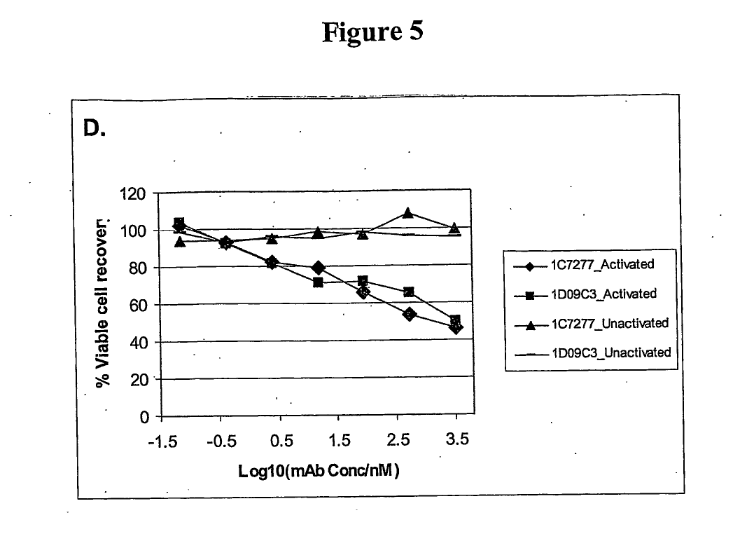

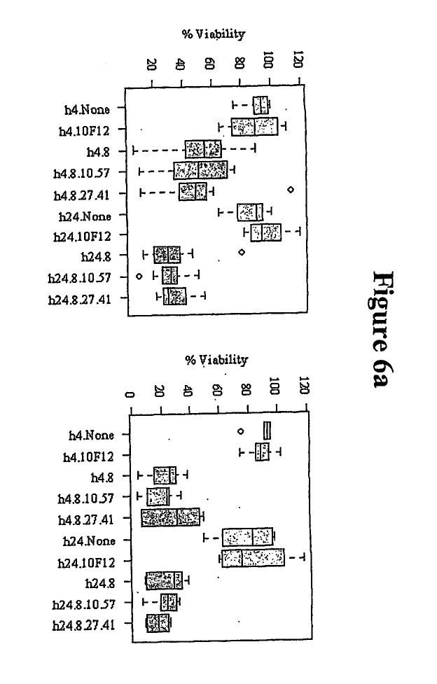

Non-adherently growing MHH preB1 cells were cultivated in RPMI medium containing the following additives (all from Gibco BRL and Bio Whittaker): 10% FCS, 2 mM L-glutamine, 1% non-essential amino acids, 1 mM sodium pyruvate and 1× Kanamycin. Aliquots were activated to increase expression of HLA-DR molecule by incubation for one day with Lipopolysaccharide (LPS, 10 μg/ml), Interferon-gamma (IFN-γ, Roche, 40 ng/ml) and phyto-hemagglutinin (PHA, 5 μg/ml). The cell surface expression of HLA-DR molecules was monitored by flow cytometry with the FITC-conjugated mAb L243 (BD Immunocytometry Systems, San Jose, Calif., USA). Incubation of MHH preB1 for one day in the presence of LPS, IFN-γ and PHA resulted in a 2-fold increase in HLA-DR surface density (mean fluorescence shift from 190 to 390). Cell killing was performed for 4 hrs in the above medium but containing a reduced FCS concentration (2.5%). A concentration series of the IgG forms of MS-GPC-8-27-41/1D09C3 & MS-GPC-8-10-57 /1C7277 was employed, consisting of final antibody concentrations of 3300, 550, 92, 15, 2.5, 0.42 and 0.07 nM, on each of an aliquot of non-activated and activated cells. Viable cells were identified microscopically by exclusion of Trypan blue. Whereas un-activated cell viability remains unaffected by the antibody up to the highest antibody concentration used, cell viability is dramatically reduced with increasing antibody concentration in activated MHH PreB1 cells (FIG. 5).

In addition, we found that cell proliferation was apparently not needed, since tumor cells in mitosis-arrest remained susceptible to mAb-mediated killing (data not shown).

In contrasts to the mAbs we describe here, two additional anti-HLA-DR mAbs with therapeutic potential, Lym-1 (Epstein et al., Cancer Res. 47:830-840, 1987; DeNardo et al., Int. J. Cancer 96 (suppl. 3):96, 1988) and 1D10 (Gingrich et al., Blood 75:2375-2387, 1990), achieve selectivity in a different way. These two mAbs recognize what appear to be posttranslational modifications on DR molecules that occur preferentially in B-cell derived tumors, although some expression was noted also on normal B cells and monocytes (Epstein et al., 1987; DeNardo et al., 1988). Neither of these mAbs has inherent tumoricidal activity, and thus, Lym-1 is developed in a ¹³¹I-labelled form (Oncolym®), whereas the efficacy of 1D10 relies on intact immunological effector mechanisms of the patient, similarly to other mAbs (Vose et al., J. Clin. Oncol. 19:389-397, 2001; Dyer et al., Blood 73:1431-1439, 1989) already available for the clinic. Furthermore, Lym-1 is a murine mAb with substantial immunogenicity for humans, and 1D10 is a humanized murine mAb. Our fully human mAbs with strong inherent tumoricidal activity and selectivity for activated/tumor transformed cells demonstrate a substantially different profile and mechanism of action from these two mAbs, and thus promise a novel therapeutic approach to lymphoma/leukemia.

13. Killing Efficiency of Human Anti-HLA-DR IgG Antibodies Against Ex-Vivo Chronic Lymphoid Leukemia Cells

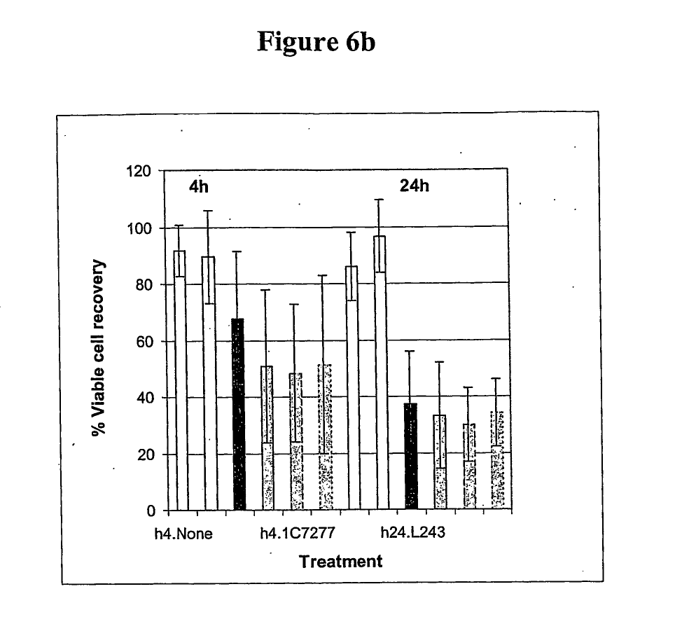

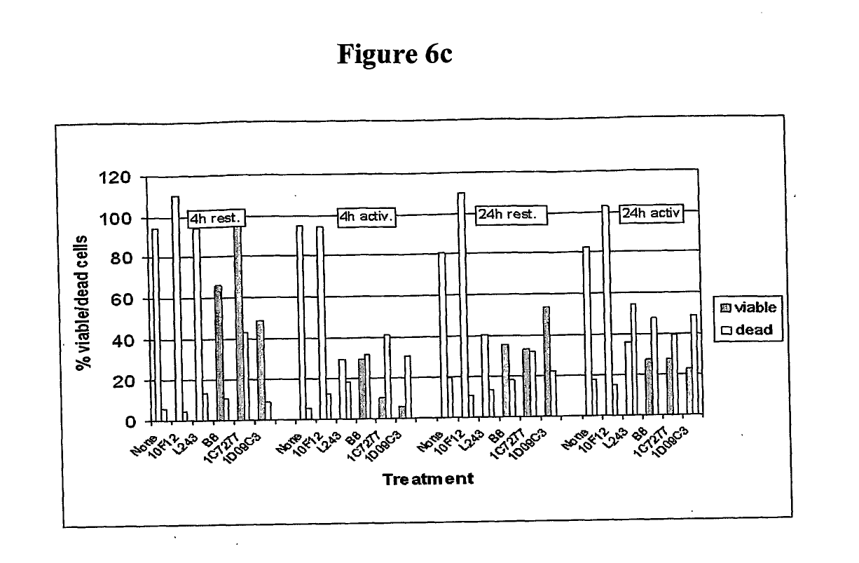

We investigated whether the human anti-DR mAbs Would also be active on freshly isolated leukemic cells, in addition to established cell lines. Using purified malignant B cells obtained from the peripheral blood of 10 un-typed chronic lymphoid leukemia (CLL) patients (Buhmann et al., Blood 93:1992-2002, 1999), we demonstrated that IgG forms of anti-HLA-DR antibody fragments of the invention showed efficacy in killing of clinically relevant cells using an ex-vivo assay (FIG. 6). Although the killing kinetics are slightly slower than those of in vitro experiments using established cell lines, significant killing is achieved over 24 hours of Ab incubation, despite the low rate of CLL cell proliferation.

B-cells were isolated and purified from 10 unrelated patients suffering from CLL (samples kindly provided by Prof Hallek, Ludwig Maximillian University, Munich) according to standard procedures (Buhmann et al., (1999)). 2×10⁵ cells were treated with 100 nM of IgG forms of the anti-HLA-DR antibody fragments MS-GPC-8, MS-GPC-8-10-57 or MS-GPC-8-27-41 and incubated for 4 or 24 hours analogous to examples 8 and 9. A replica set of cell cultures was established and activated by incubation with HeLa-cells expressing CD40 ligand on their surface for three days before treatment with antibody (Buhmann et al., 1999). As controls, the murine IgG 10F12 (Vidovic et al., 1995b) or no antibody was used. Cell viability for each experiment was determined as described in example 12.

Surprisingly, IgG forms of the anti-HLA-DR antibody fragments of the invention showed highly efficient and uniform killing—even across this diverse set of patient material. After only 4 hours of treatment, all three human IgGs gave a significant reduction in cell viability compared to the controls, and after 24 hours only 33% of cells remained viability (FIG. 6). We found that on stimulating the ex-vivo cells further according to Buhmann et al. (1999), the rate of killing was increased such that after only 4 hours culture with the human antibodies, only 24% of cells remained viable on average for all patient samples and antibody fragments of the invention. The control murine anti-DR mAb 10F12, which has no inherent tumoricidal activity (Vidovic, D. et al., Eur. J. Immunol. 25:3349-3355, 1995), had no effect on CLL cells (FIG. 6c).

14. Determination of EC₅₀ for Anti-HLA-DR Antibody Fragments

We demonstrated superior Effective Concentration at 50% effect (EC₅₀) values in a cell-killing assay for certain forms of anti-HLA-DR antibody fragments selected from the HuCAL library compared to cytotoxic murine anti-HLA-DR antibodies (Table 6).

The EC₅₀ for anti-HLA-DR antibody fragments selected from the HuCAL library were estimated using the HLA-DR positive cell line PRIESS or LG2 (ECACC, Salisbury UK). 2×10⁵ cells were incubated for 4 h at 37° C. under 6% CO₂ in RPMI 1640 (PAA, Germany) supplemented with 2.5% heat inactivated FBS (Biowhittaker Europe, BE), 2 mM L-glutamine, 1% non-essential amino acids, 1 mM sodium pyruvate and 0.1 mg/ml kanamycin, together with dilution series of bivalent anti-HLA-DR antibody fragments. For the dilution series of Fab antibody fragments, an appropriate concentration of Fab fragment and anti-FLAG M2 antibody were premixed to generate bivalent compositions of the anti-HLA-DR antibody fragments. The concentrations stated refer to the concentration of bivalent composition such that the IgG and Fab EC₅₀ values can be compared.

After 4 h incubation with bivalent antibody fragments at 37° C. under 6% CO₂, cell viability was determined by fluorescein diacetate staining and subsequent counting of remaining viable cells (Current Protocols in Immunology, 1997). Using standard statistical software, non-linear logistic regression curves were fitted to replica data points and the EC₅₀ estimated for each antibody fragment.

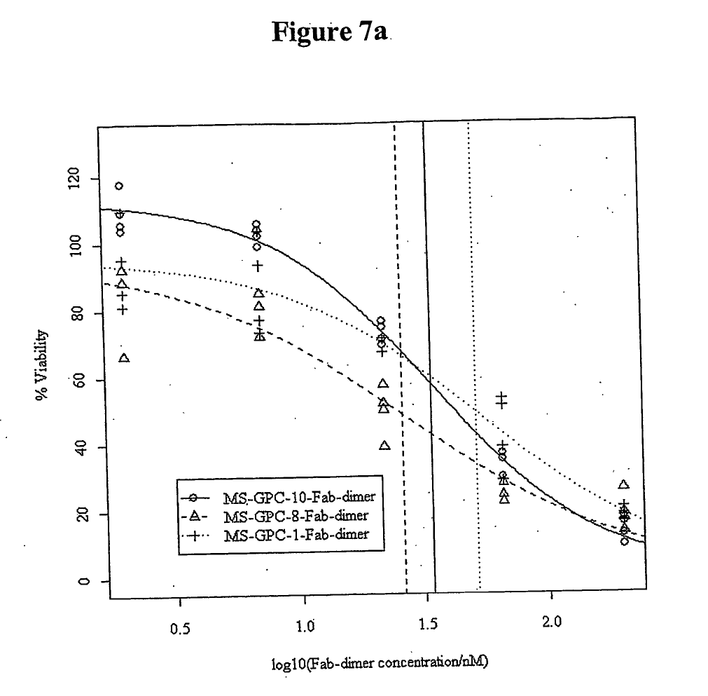

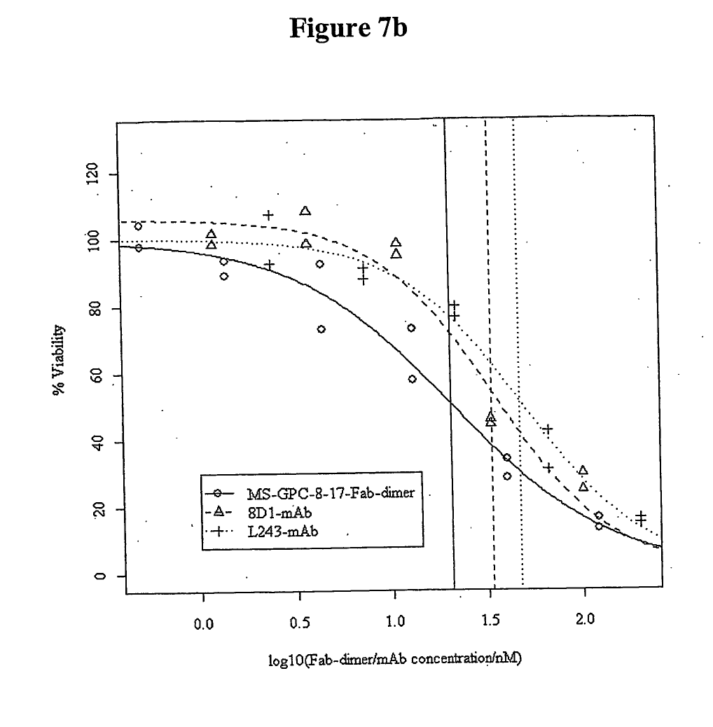

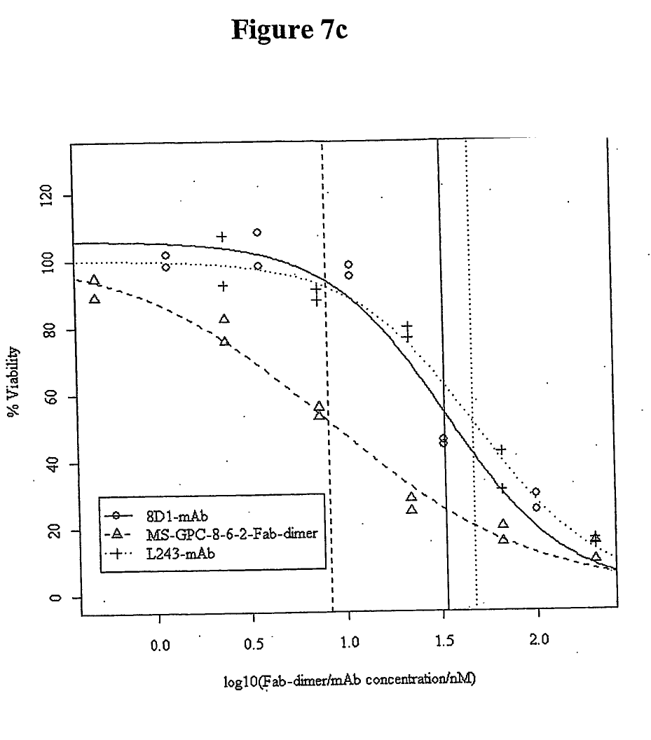

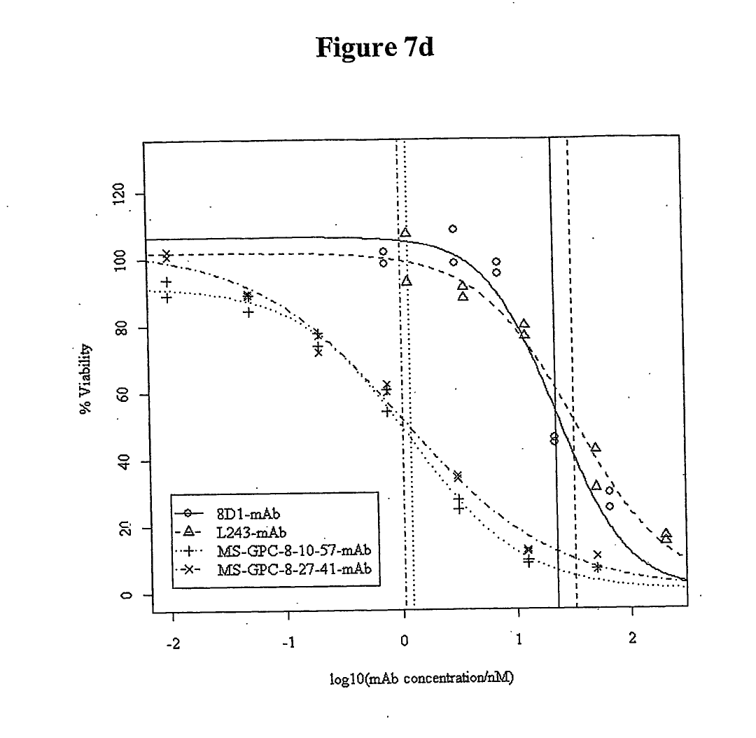

When cross-linked using the anti-FLAG M2 antibody, the Fab fragments MS-GPC-1, MS-GPC-8 & MS-GPC-10 selected from the HuCAL library (Example 4) showed an EC₅₀ of less than 120 nM as expressed in terms of the concentration of the monovalent fragments, which corresponds to a 60 nM EC₅₀ for the bivalent cross-linked (Fab)dimer-anti-Flag M2 conjugate. (FIG. 7a). When cross-linked using the anti-FLAG M2 antibody, anti-HLA-DR antibody fragments optimised for affinity within the CDR3 region (example 4) showed a further improved EC₅₀ of less than 50 nM, or 25 nM in terms of the bivalent cross-linked fragment (FIG. 7b), and those additionally optimised for affinity within the CDR1 region showed an EC₅₀ of less than 30 nM (15 nM for bivalent fragment). In comparison, the EC₅₀ of the cytotoxic murine anti-HLA-DR antibodies 8D1 (Vidovic & Toral; 1998) and L243 (Vidovic et al; 1995b) showed an EC₅₀ of over 30 and 40 nM, respectively, within the same assay (FIG. 7c).

Surprisingly, the IgG form of certain antibody fragments of the invention showed approximately 1.5 orders of magnitude improvement in EC₅₀ compared to the murine antibodies (FIG. 7d). For example, the IgG forms of MS-GPC-8-10-57 & MS-GPC-8-27-41 showed an EC₅₀ of 1.2 and 1.2 nM respectively. Furthermore, despite being un-optimised for affinity, the IgG form of MS-GPC-8 showed an EC₅₀ of less than, 10 nM.

As has been shown in examples 11 and 12, the efficiency of killing of un-activated cells (normal peripheral B and MHH PreB cells respectively) is very low. After treatment with 50 nM of the IgG forms of MS-GPC-8-10-57 & MS-GPC-8-27-41, 78% and 83% of normal peripheral B cells, respectively, remain viable after 4 hours. Furthermore, at only 50 nM concentration or either IgG, virtually 100% viability is seen for MHH PreB1 cells. Indeed, a decrease in the level of viability to below 50% cannot be achieved with these un-activated cells using reasonable concentration ranges (0.1 to 300 mM) of IgG or bivalent, cross-linked Fab forms of the anti-HLA-DR antibody fragments of the invention. Therefore, the EC₅₀ for these un-activated cell types can be estimated to be at least 5 times higher than that shown for the non-optimised Fab forms (EC₅₀ 60 nM with respect to cross-linked bivalent fragment), and at least 10 times and 100 times higher than EC₅₀s shown for the VHCDR3 optimised Fabs (−25 nM with respect to cross-linked bivalent fragment) and IgG forms of MS-GPC-8-10-57 (−1.2 nM) & MS-GPC-8-27-41 (−1.2 nM) respectively.

15. Mechanism of Cell-Killing

The examples described above show that cell death occurs—needing only certain multivalent anti-HLA-DR antibody fragments to cause killing of activated cells. No further cytotoxic entities or immunological mechanisms were needed to cause cell death, therefore demonstrating that cell death is mediated through an innate pre-programmed mechanism of the activated cell. The mechanism of apoptosis is a widely understood process of pre-programmed cell death. We were surprised by certain characteristics of the cell killing we observed that suggested the mechanism of killing for activated cells when exposed to our human anti-HLA-DR antibody fragments was not what is commonly understood in the art as “apoptosis”. For example, the observed rate of cell killing appeared to be significantly greater than the rate reported for apoptosis of immune cells (about 10-15 hrs; Truman et al., 1994). Two experiments were conducted to demonstrate that the mechanism of cell killing proceeded by a non-apoptotic mechanism.

First, we used Annexin-V-FITC and propidium iodide (PI) staining techniques to distinguish between apoptotic and non-apoptotic cell death—cells undergoing apoptosis, “apoptotic cells”, (Annexin-V positive/PI negative) can be distinguished from necrotic (“Dead”) (Annexin-V positive/PI positive) and fully functional cells (Annexin-V negative/PI negative). Using the procedures recommended by the manufacturers of the AnnexinV and PI assays, 1×10⁶/ml PRIESS cells were incubated at 37° C. under 6% CO₂ with or without 200 nM anti-HLA-DR antibody fragment MS-GPC-8 together with 100 nM of the cross-linking anti-FLAG M2 mAb in RPMI 1640 (PAA, DE) supplemented with 2.5% heat inactivated FCS (Biowhittaker Europe, BE), 2 mM L-glutamine, 1% non-essential amino acids, 1 mM sodium pyruvate and 0.1 mg/ml kanamycin. To provide an apoptotic cell culture as control, 1×10⁶/ml PRIESS cells were induced to enter apoptosis by incubation in the above medium at 37° C. under 6% CO₂ with 50 μg/ml of the apoptosis-inducing anti-CD95 mAb DX2 (BD Pharmingen, Torrey Pine, Calif., USA) cross-linked with 10 μg/ml Protein-G. At various incubation times (1, 15 and 60 min., 3 and 5 hrs) 200 μl samples were taken, washed twice and stained with Annexin-V-FITC (BD Pharmingen, Torrey Pine, Calif., USA) and PI using Annexin-V binding buffer following the manufacturer's protocol. The amount of staining with Annexin-V-FITC and PI for each group of cells is analysed with a FACS Calibur (BD Immunocytometry Systems, San Jose, Calif., USA).

Cell death induced through the cross-linked anti-HLA-DR antibody fragments shows a significantly different pattern of cell death than that of the anti-CD95 apoptosis inducing antibody or the cell culture incubated with anti-FLAG M2 mAb alone. The percentage of dead cells (as measured by Annexin-V positive/PI positive staining) for the anti-HLA-DR antibody fragment/anti-FLAG M2 mAb treated cells increases far more rapidly than that of the anti-CD95 or the control cells (FIG. 8a). In contrast, the percentage of apoptotic cells (as measured by Annexin-V positive/PI negative staining) increases more rapidly for the anti-CD95 treated cells compared to the cross-linked anti-HLA-DR antibody fragments or the control cells (FIG. 8b).

Second, we inhibited caspase activity using zDEVD-fmk, an irreversible Caspase-3 inhibitor, and zVAD-fink, a broad spectrum Caspase inhibitor (both obtained from BioRad, Munich, Del.). The mechanism of apoptosis is characterized by activity of caspases, and we hypothesized that if caspases were not necessary for anti HLA-DR mediated cell death, we would observe no change in the viability of cells undergoing cell death in the presence of these caspase inhibitors compared to those without. 2×10⁵ PRIESS cells were preincubated for 3 h at 37° C. under 6% CO₂ with serial dilutions of the two caspase inhibitors ranging from 180 μM to 10 mM in RPMI 1640 (PAA, DE) supplemented with 2.5% heat inactivated FCS (Biowhittaker Europe, BE), 2 mM L-glutamine, 1% non-essential amino acids, 1 mM sodium pyruvate and 0.1 mg/ml kanamycin. HLA-DR mediated cell death was induced by adding 200 nM of the human anti-HLA-DR antibody fragment MS-GPC-8 and 100 nM of the cross-linking anti-M2 mAb. An anti-CD95 induced apoptotic cell culture served as a control for the activity of inhibitors (Drenou et al., 1999). After further incubation at 37° C. and 6% CO₂, cell viability after 4 and 24 h was determined by trypan blue staining and subsequent counting of non-stained cells. As we expected, cell viability of the anti-HLA-DR treated cell culture was not significantly modified by the presence of the Caspase inhibitors, while cell death induced through anti-CD95 treatment was significantly decreased for the cell culture pre-incubated with the Caspase inhibitors. We therefore concluded that the cell death induced by the human anti-DR mAbs does not occur via the classical apoptotic pathway that can be inhibited by zDEVD-fm or zVAD-fink.

16. In Vivo Therapy for Cancer Using an MA-DR Specific Antibody

To test the in vivo efficacy, we inoculated immunocompromised (such as scid, nude or Rag-1 knockout) SCID (severe combined immunodeficient) mice subcutaneously (s.c.) or intravenously (i.v.) with the non-Hodgkin B cell lymphoma line GRANTA-519 (see in Table 5), and monitored tumor development in mice treated with mAb, in comparison to solvent-treated animals.

In general, mice are treated i.v. or s.c with the IgG form of the anti-HLA-DR antibody fragments MS-GPC-8, MS-GPC-8-10-57, MS-GPC-8-27-41 or others of the invention prepared as described above, using doses of 1 to 25 mg/kg over 5 days. Survival of anti-HLA-DR treated and control untreated mice is monitored for up to 8 weeks after cessation of treatment. Tumor progression in the mice inoculated s.c. is additionally quantified by measuring tumor surface area.

For example, eight weeks old female C.B.-17 scid mice were injected with anti-asialoGM1 antibody (Wako Chemicals, Neuss, Germany; 25 μl diluted 4 fold in PBS, i v.) to suppress natural killer (NK) cell activity, on days 0, 1, and 2. On day 1, 5×10⁶ GRANTA-519 cells were injected s.c. into the right flank, or i.v. The endpoint in the s.c. model is a tumor surface area of >5 cm², skin ulceration above the tumor, or death, and in the i.v. model hind leg paralysis or death. Mice were treated with 1 mg or 0.2 mg 1D09C3 mAb s.c. or i.v. on days 5, 7 and 9. Control mice received PBS. Mice were monitored, and tumor length and width were measured by a slide-gauge twice a week.

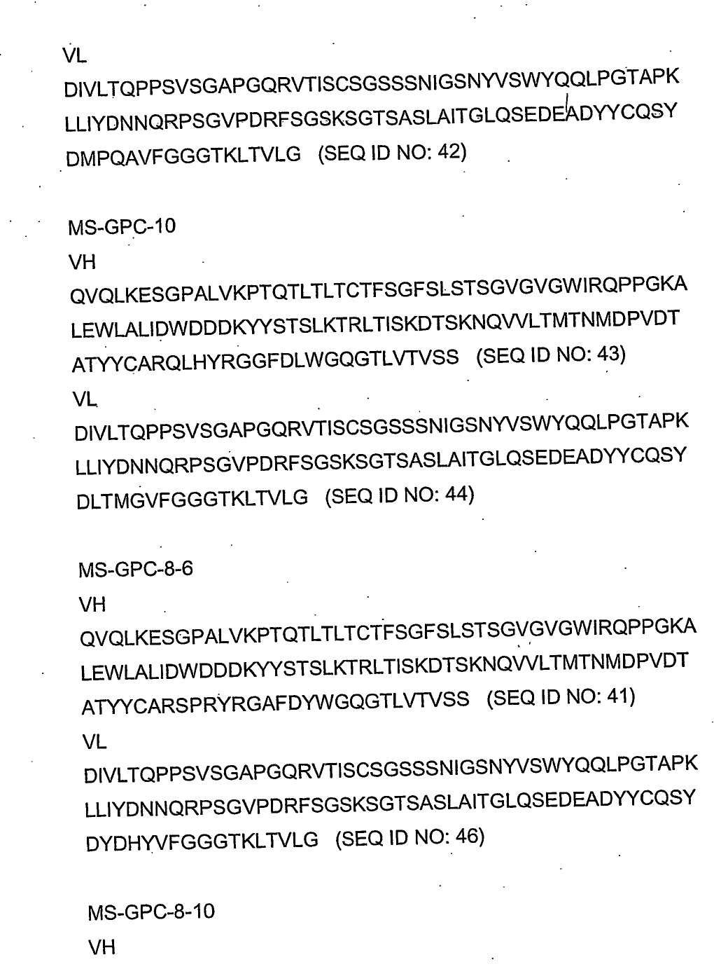

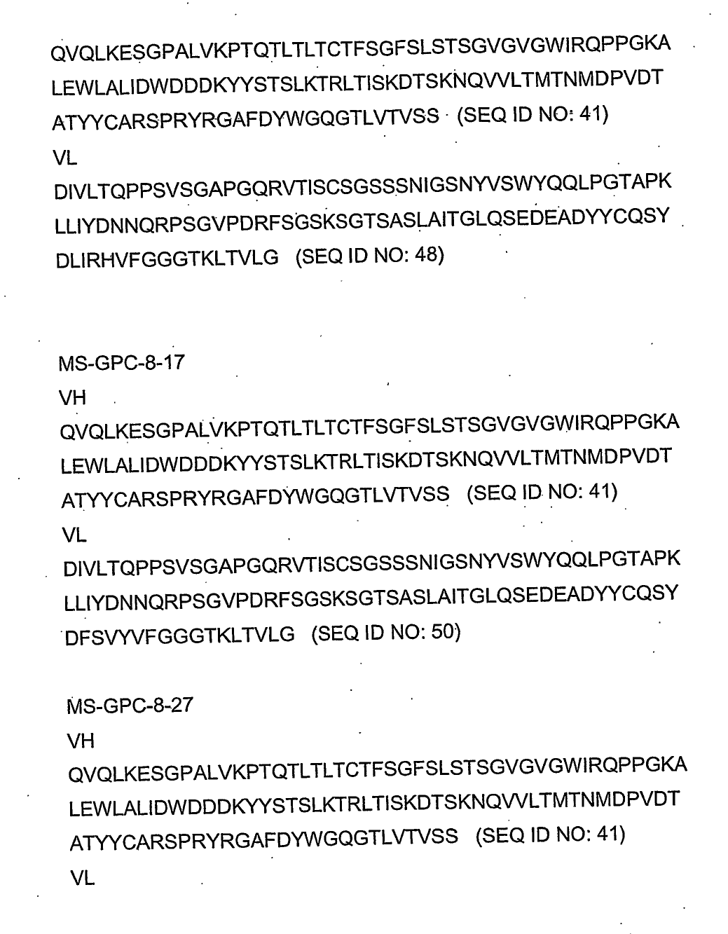

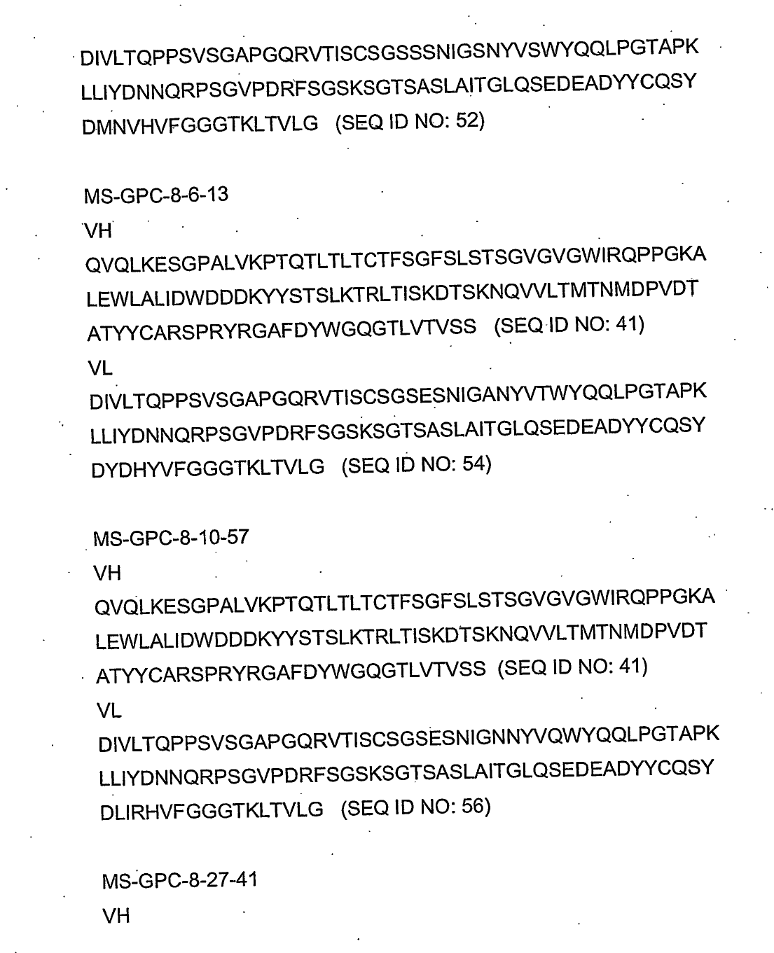

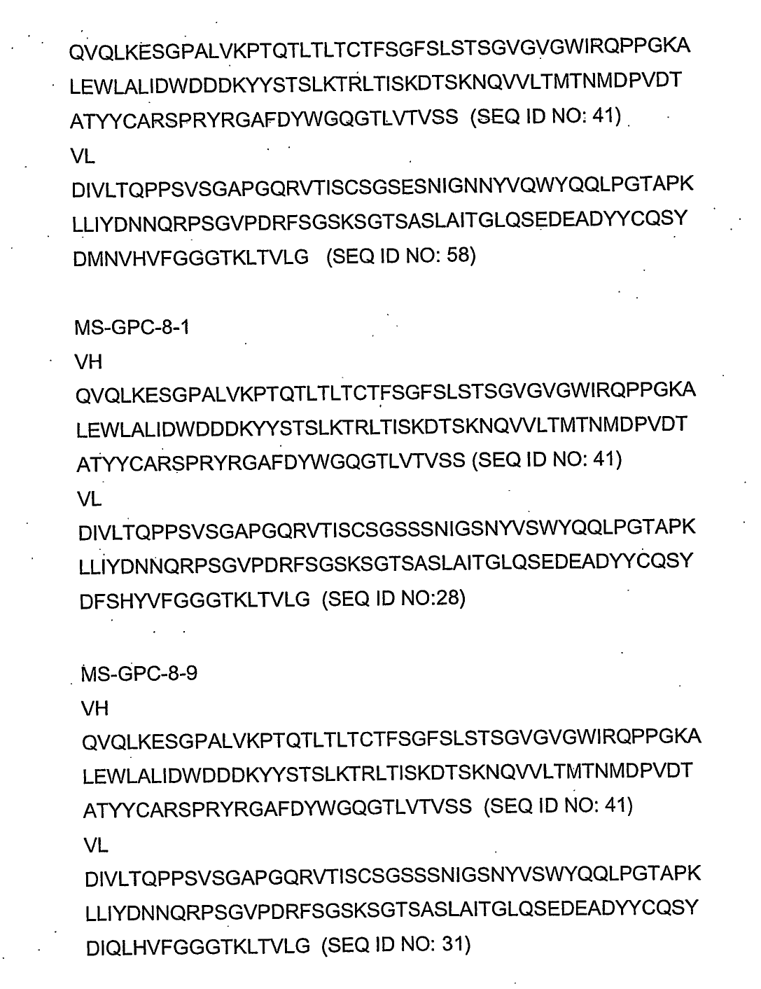

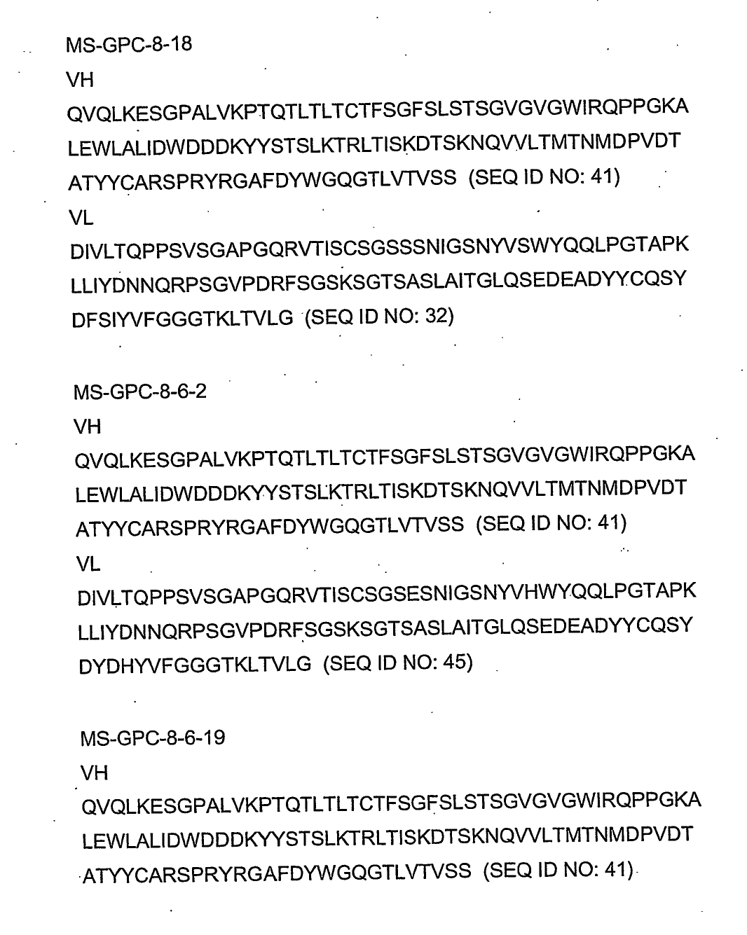

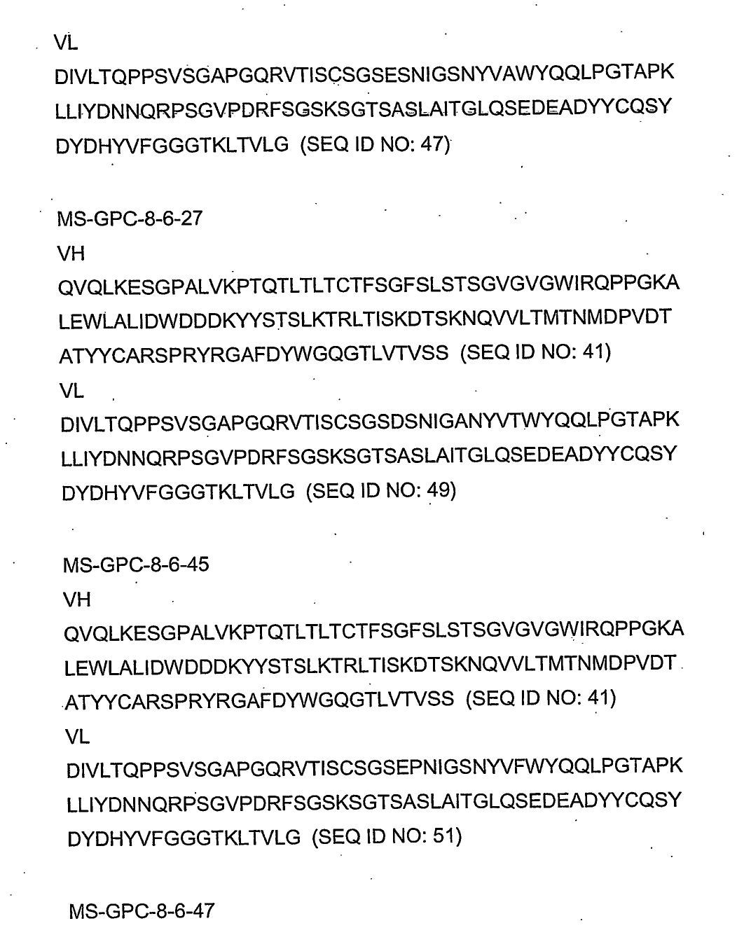

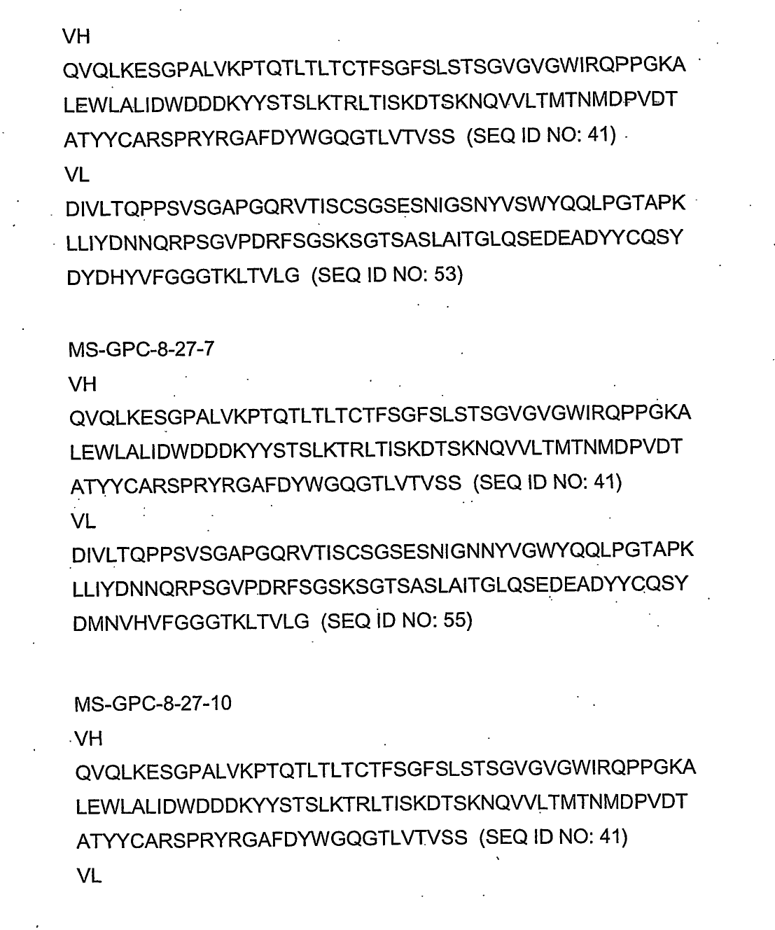

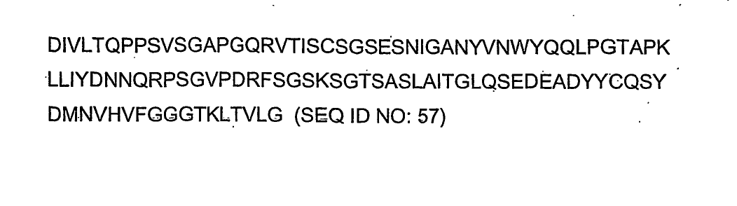

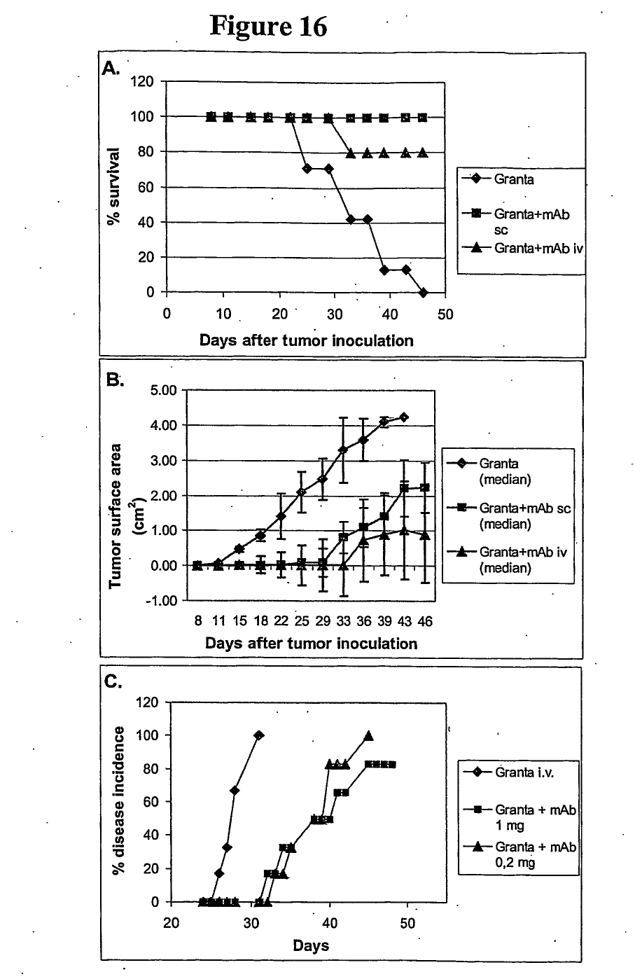

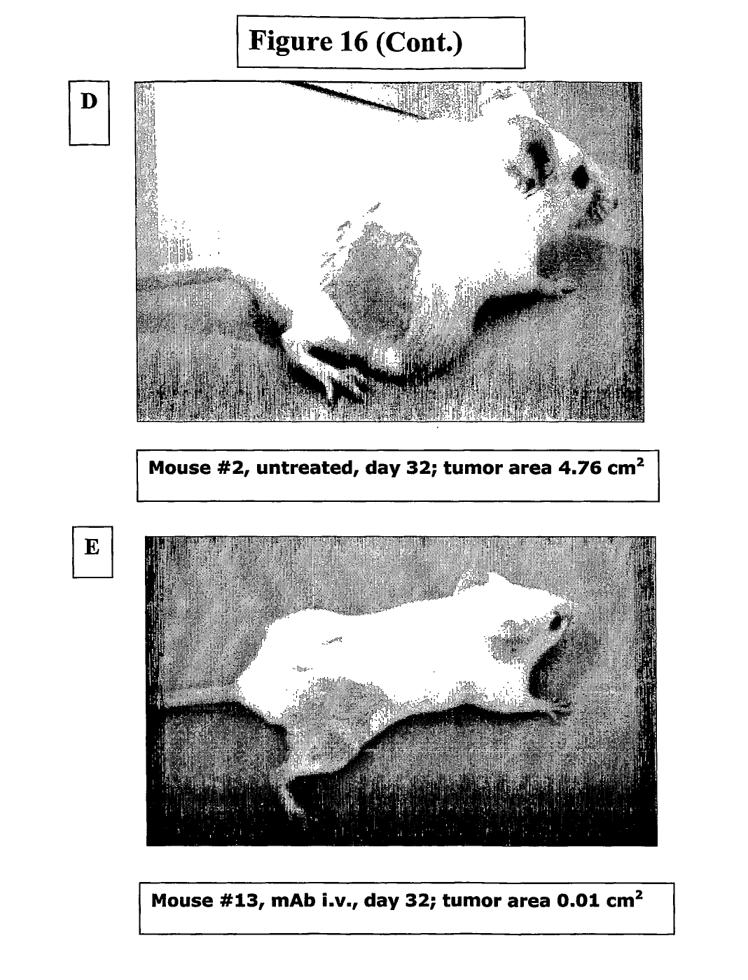

Significant prolongation of survival of up to 80% of anti-HLA-DR treated mice is observed during the experiment, and up to 50% mice survive at the end of the experiment. In the s.c. tumor experiment, at day 48, 100% of s.c. mAb treated mice were alive and 80% of i.v. mAb treated mice were alive (death is not related to mAb treatment or tumor), while all control mice died within the observation period (FIG. 16a). In s.c. inoculated and untreated mice, the tumor reaches a surface area of 2-3 cm², while in anti-HLA-DR treated animals the tumor surface area is significantly less. FIG. 16d shows representative tumor size in mice treated or untreated by mAb of the instant invention. Tumor growth was also significantly retarded in the treated animals (FIG. 16b). In the i.v. tumor experiment, a significant delay (about 30 days) in disease onset was observed in the mAb treated groups (FIG. 16c). The 30 day survival rate for i.v. mAb treated mice is 100%, while the survival rate for control mice is 0%. Even at day 40, the survival rate for i.v. mAb treated mice is 50%/20% (for high/low doses, respectively). Tumor-induced paralysis is also significantly reduced in the iv. mAb treated mice as compared to the control group mice which are all paralysized by day 40.

These experiments demonstrate that human antigen-binding domains n can successfully be used as a therapeutic for the treatment of cancer. The in vitro, ex vivo and in vivo efficacy data presented here are strong evidence that such mAbs offer the potential to become useful and potent therapeutic agents for the treatment of different DR+lymphoma and leukemia

17. Immunosuppression Using Anti-HLA-DR Antibody Fragments Measured by Reduction in IL-2 Secretion

Various diseases are caused by or associated with activated T-cells. For example, delayed-type hyper sensitivity (DTH) is caused by T-cells activated by antigen-presenting cells (APCs) via MHC receptors. Thus, inhibition of interaction between the MHC class II molecule and the T-cell receptor (TCR) can inhibit certain undesirable immune responses.

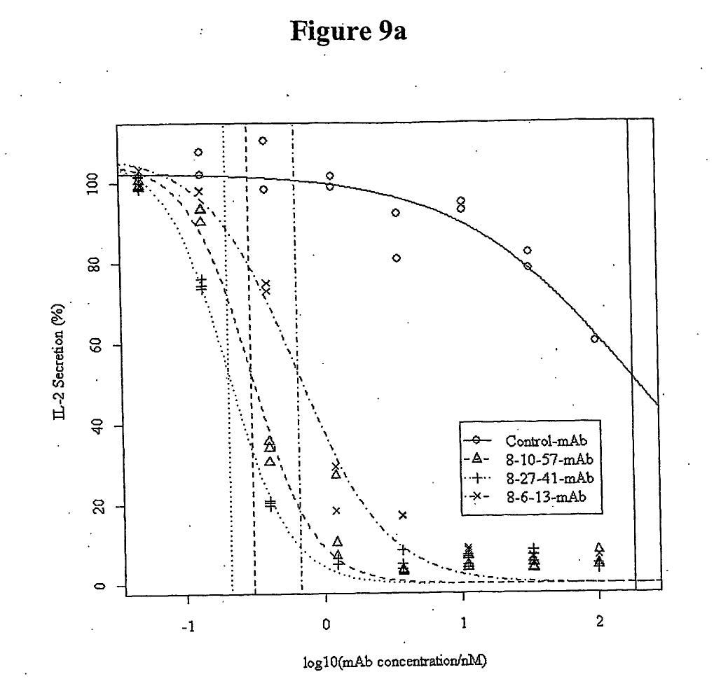

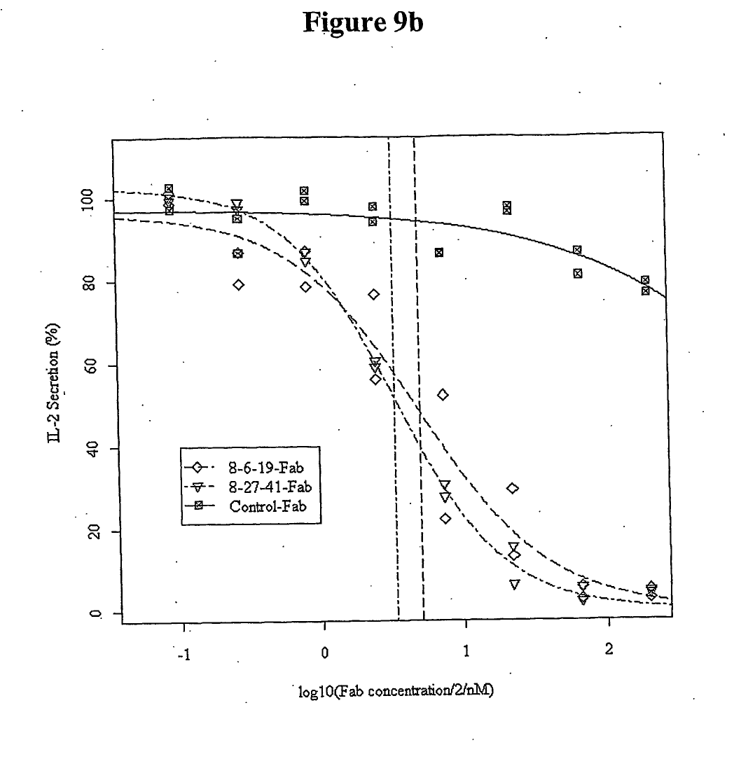

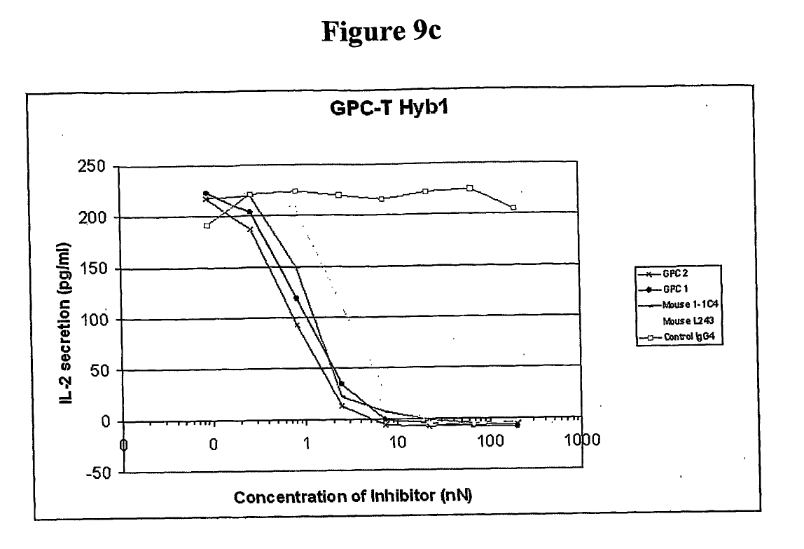

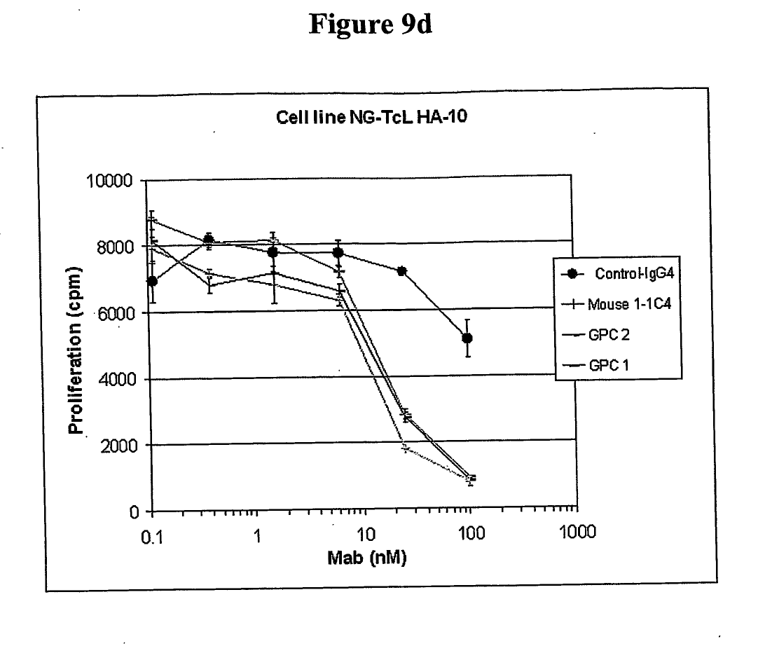

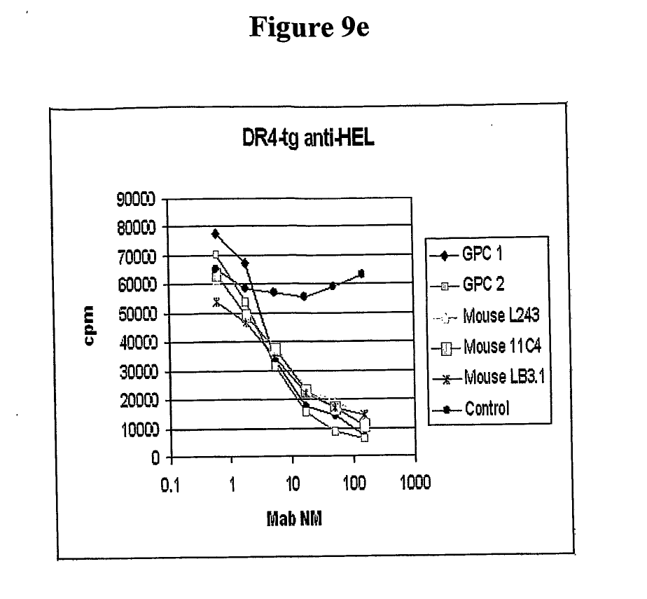

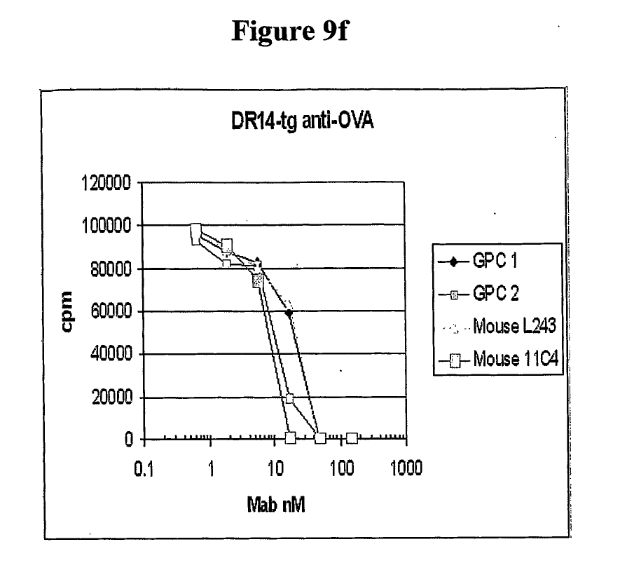

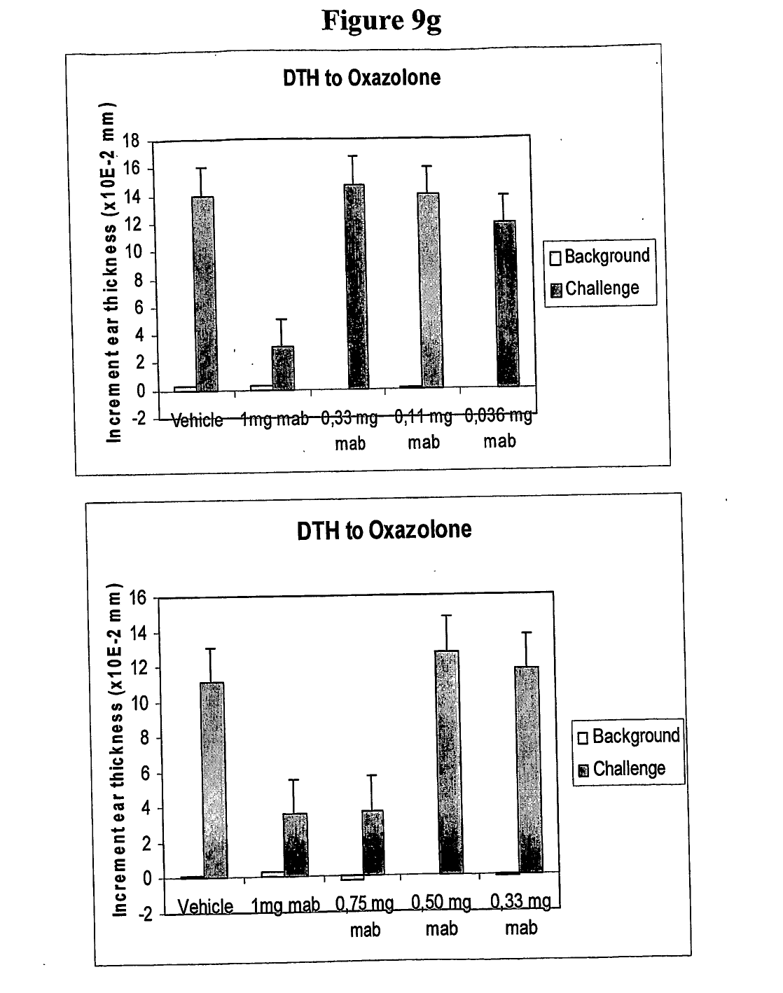

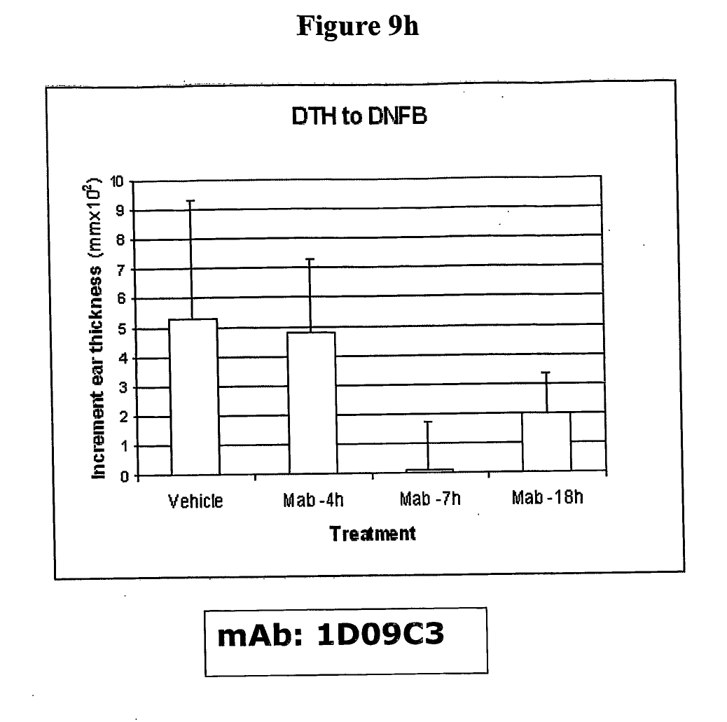

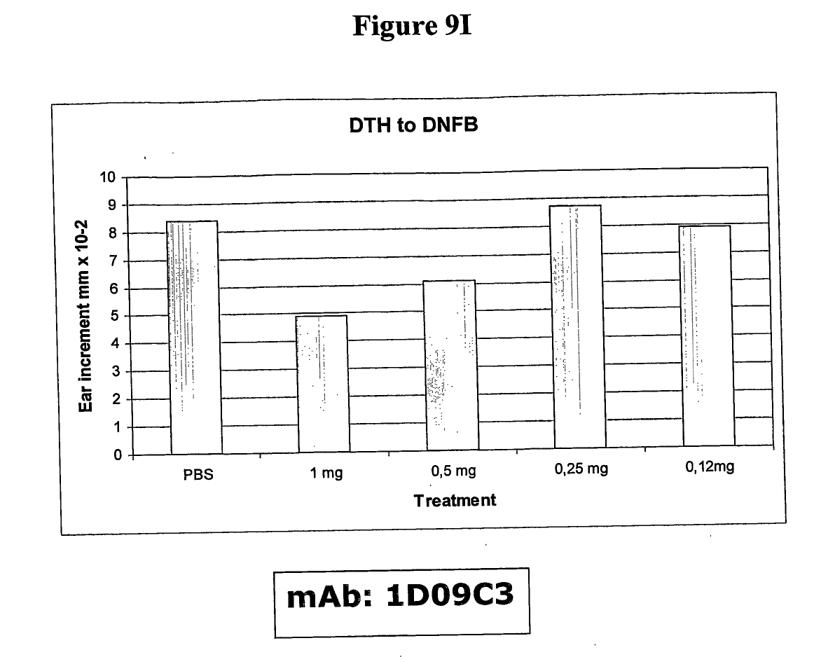

We were surprised to observe that certain anti-HLA-DR antibody fragments of the invention also displayed substantial immunomodulatory properties within an assay measuring IL-2 secretion from immortalized T-cells (T-cell hybridoma). IgG forms of the antibody fragments MS-GPC-8-6-13/305D3, MS-GPC-8-10-57/1C7277 & MS-GPC-8-27-41/1D09C3 showed very strong immunosuppressive properties in this assay with sub-nanomolar IC₅₀ values and virtually 100% maximal inhibition (FIG. 9a). Particularly surprising was our observation that certain monvalent compositions of the antibody fragments of the invention were able to strongly inhibit IL-2 secretion in the same assay. For example, Fab forms of the VH CDR3-selected and VL CDR3/VL CDR1 optimised antibody fragments showed low single-digit nM IC₅₀'s and also almost 100% maximal inhibition (FIG. 9b). Other monvalent anti-HLA-DR antibody fragments of the invention showed significant immunosuppressive properties in the assay compared to control IgG and Fab fragments (Table 7). FIG. 9c also shows immunomodulatory properties of the mouse 1-2 C4 and L243 mAb as well as the GPC1 and 2 Ab's.

The immunomodulatory properties of anti-HLA-DR antibody fragments was investigated by measuring IL-2 secretion from the hybridoma cell line T-Hyb1 stimulated using DR-transgenic antigen presenting cells (APC) under conditions of half-maximal antigen stimulation. IL-2 secretion was detected and measured using a standard ELISA method provided by the OptiEIA mouse IL-2 kit of Pharmingen (Torrey Pine, Calif., USA). APCs were isolated from the spleen of unimmunized chimeric 0401-IE transgenic mice (Ito et al. 1996) according to standard procedures. 1.5×10⁵ APCs were added to 0.2 ml wells of 96-well in RPMI medium containing the following additives (all from Gibco BRL and PAA): 10% FCS, 2 mM L-glutamine, 1% non-essential amino acids, 1 mM sodium pyruvate and 0.1 g/l kanamycin. Hen egg ovalbumin was added to a final concentration of 200 μg/ml in a final volume of 100 ul of the above medium, the cells incubated with this antigen for 30 min at 37° C. under 6% CO₂. Anti-HLA-DR antibody fragments were added to each well at various concentrations (typically in a range from 0.1 to 200 nM), the plate incubated for 1 h at 37° C./6% CO₂ and 2×10⁵ T-Hyb1 cells added to give a final volume of 200 μl in the above medium. After incubation for 24 h, 100 μl of supernatant was transferred to an ELISA plate (Nunc-Immuno Plate MaxiSorp surface, Nunc, Roskilde, DK) previously coated with IL-2 Capture Antibody (BD Pharmingen, Torrey Pine, Calif., USA), the amount of IL-2 was quantified according to the manufacturer's directions using the OptiEIA Mouse IL-2 kit and the plate read using a Victor V reader (Wallac, Finland). Secreted IL-2 in pg/ml was calibrated using the IL-2 standards provided in the kit.

The T-cell hybridoma line T-Hyb1 was established by fusion of a T-cell receptor negative variant of the thymoma line BW 5147 (ATCC) and lymph node cells from chimeric 0401-IE transgenic mice previously immunized with hen egg ovalbumin (Ito et al. 1996). The clone T-Hyb1 was selected for the assay since it responded to antigen specific stimulation with high IL-2 secretion.

18. Immunosuppression Using an HLA-DR Specific Antibody Measured by T Cell Proliferation

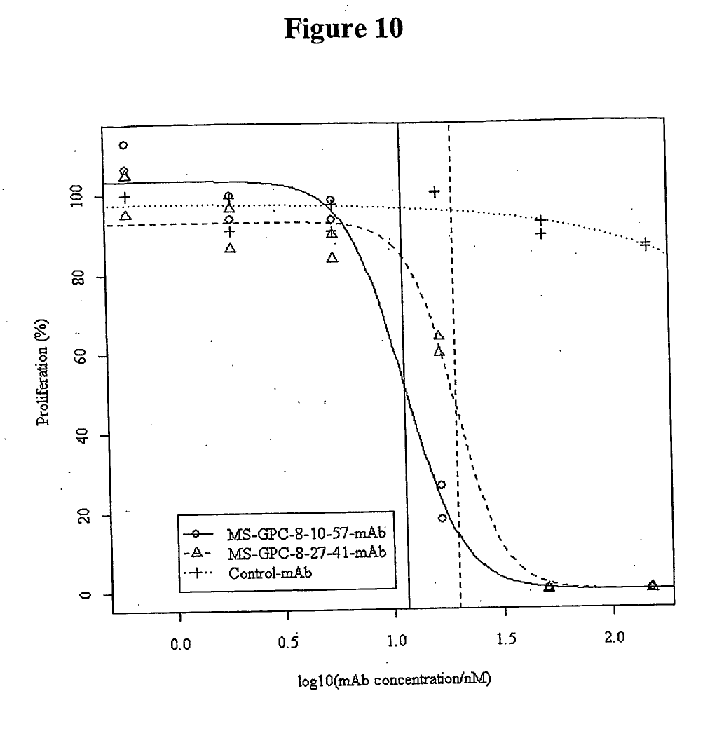

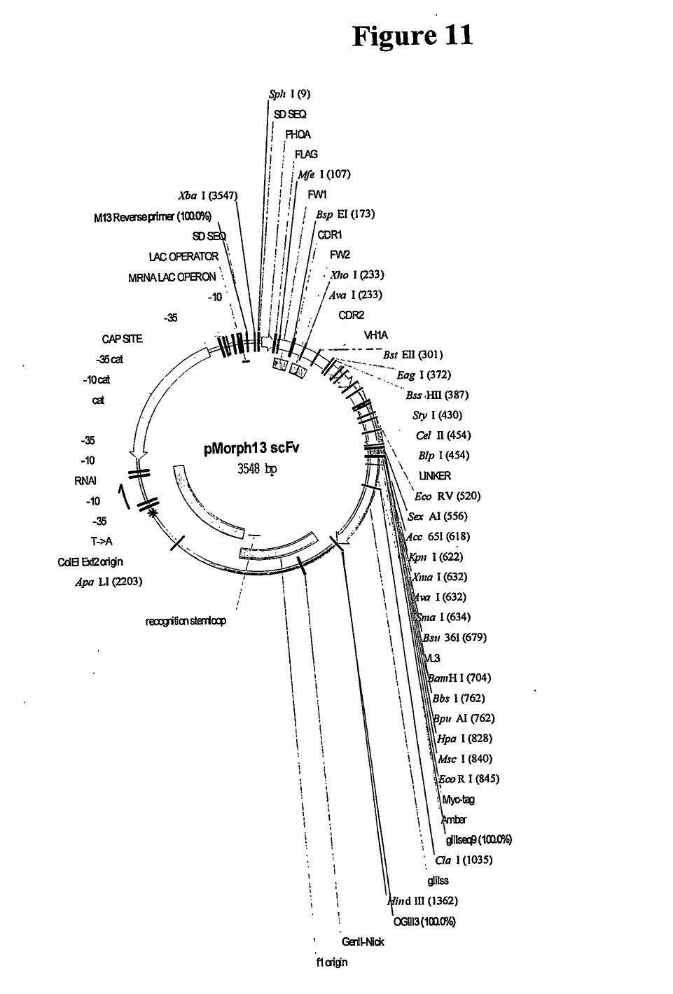

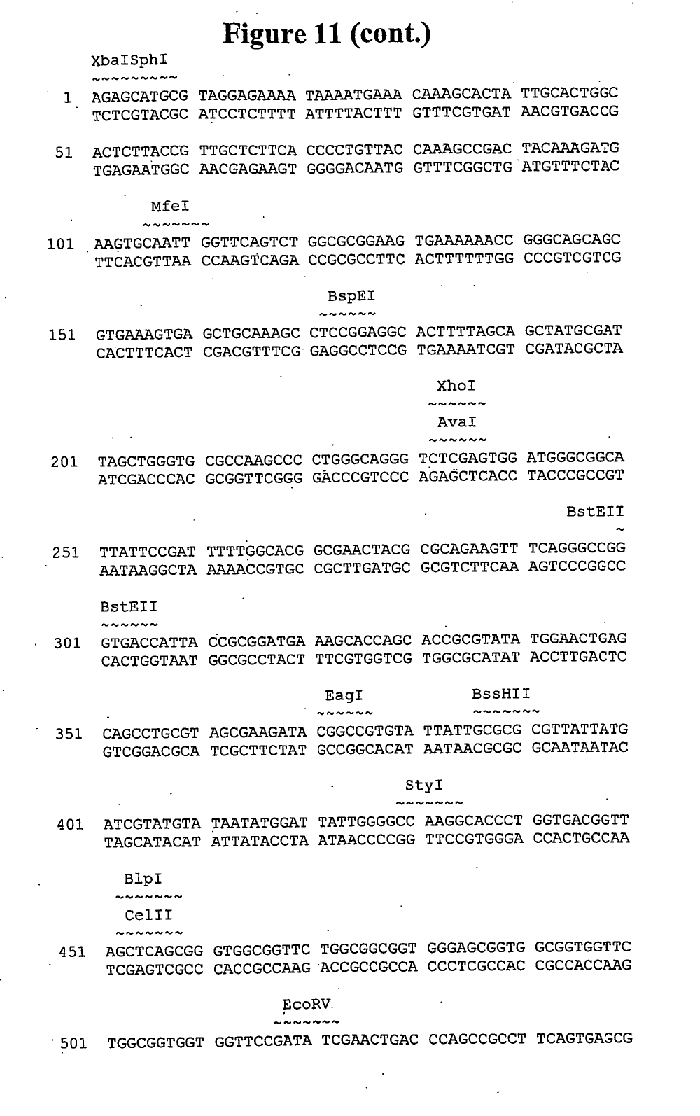

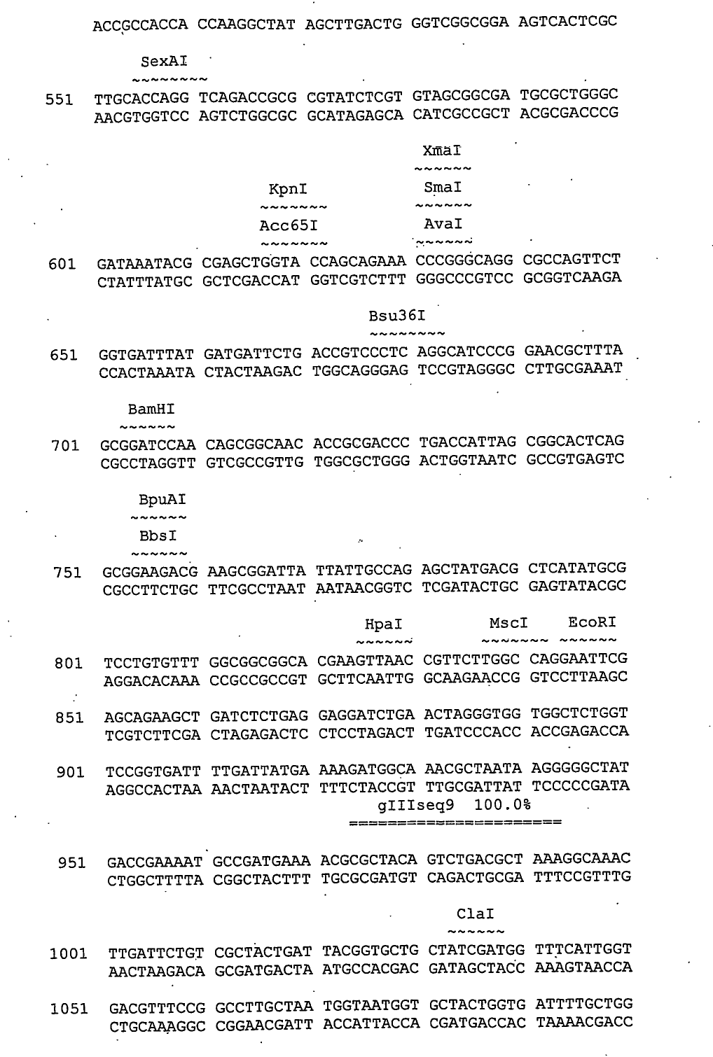

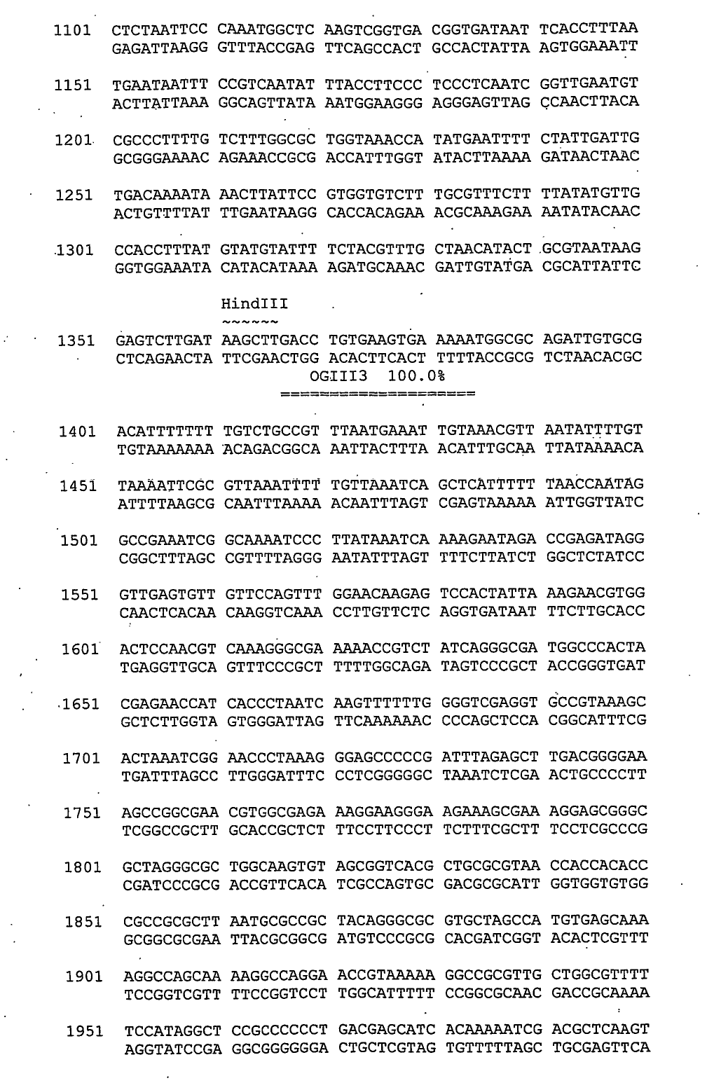

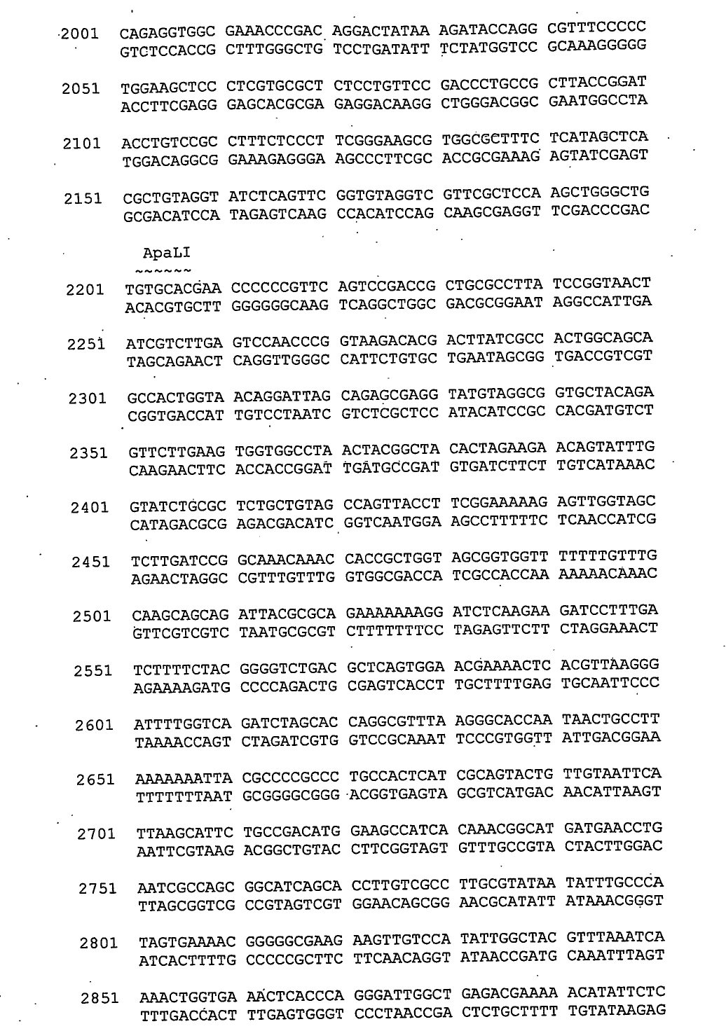

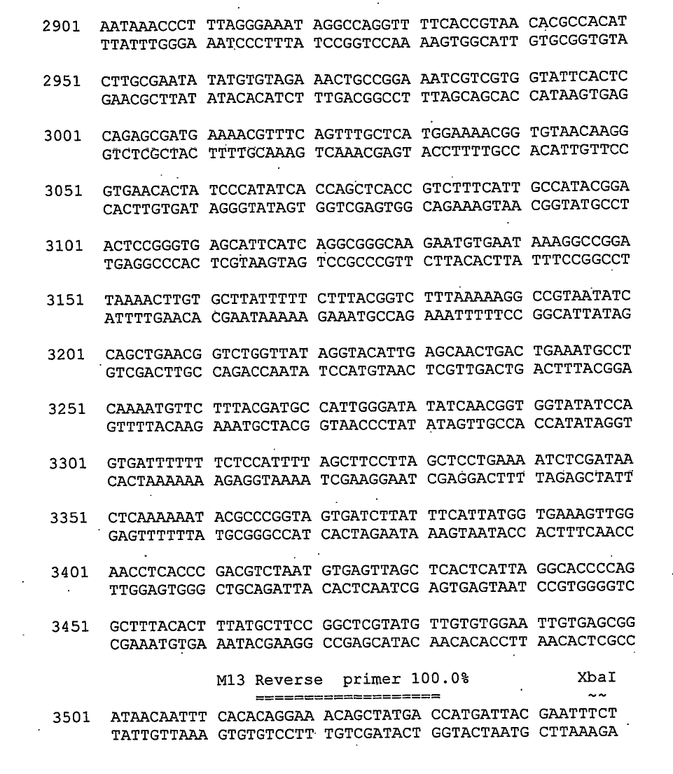

Immunomodulatory properties of the anti-HLA-DR antibody fragments were also seen within an assay that measures T cell proliferation. The IC₅₀ value for inhibition of T cell proliferation of the IgG form of MS-GPC-8-10-57/1C7277 and MS-GPC-8-27-41/1D09C3 were 11 and 20 nM respectively (FIG. 10). The anti-HLA-DR antibody fragments were tested as follows to inhibit the proliferative T cell response of antigen-primed lymph node cells from mice carrying a chimeric mouse-human class II transgene with an RA-associated peptide binding site, and lack murine class II molecules duller et al., 1990; Woods et al., 1994; Current Protocols in Immunology, Vol. 2, 7.21; Ito et al., 1996). Here, the immunization takes place in vivo, but the inhibition and readout are ex vivo. Transgenic mice expressing MHC class II molecules with binding sites of the RA associated molecule, DRB*0401 were commercially obtained. These mice lack murine MHC class II, and thus, all Th responses are channelled through a single human RA-associated MHC class II molecule (Ito et al., 1996). These transgenic mice represent a model for testing human class II antagonists.

The inhibitory effect of the anti-HLA-DR antibody fragments and their IgG forms were tested on T-cell proliferation measured using chimeric T-cells and antigen presenting cells isolated from the lymph nodes of chimeric 0401-I^{E} transgenic mice (Taconic, USA) previously immunized with hen egg ovalbumin (Ito et al., 1996) according to standard procedures. 1.5×10⁵ cells are incubated in 0.2 ml wells of 96-well tissue culture plates in the presence of ovalbumin (30 μg per well—half-maximal stimulatory concentration) and a dilution series of the anti-HLA-DR antibody fragment or IgG form under test (0.1 nM-200 nM) in serum free HL-1 medium containing 2 mM L-glutamine and 0.1 g/L Kanamycin for three days. Antigen specific proliferation is measured by ³H-methyl-thymidin (1 μCi/well) incorporation during the last 16 hrs of culture (Falcioni et al., 1999). Cells are harvested, and ³H incorporation measured using a scintillation counter (TopCount, Wallac Finland). Inhibition of T-cell proliferation on treatment with the anti-HLA-DR antibody fragment and its IgG form was observed by comparison to control wells containing antigen. FIG. 9d showed that the proliferation of the T-cell line NG-TcL HA-10 was significantly inhibited by the two GPC antibodies (MS-GPC-8-10-57/1C7277 and MS-GPC-8-27-41/1D09C3), at least to the same extent of the mouse 1-1C4 positive control Ab.

FIGS. 9e and 9f showed that transgenic T-cell proliferation as measured by ³H incorporation in two experiments were significantly inhibited by mAb treatments, including MS-GPC-8-10-57/1C7277 and MS-GPC-8-27-41/1D09C3 human mAb's and mouse L243, 11C4 and LB3.1 Ab's. In these experiments, T-cells are sensitized in vivo by specific antigens (ovalbumin (OVA) in one case, hen egg lysozyme (HEL) in another case), followed by re-stimulation ex vivo by these two antigens respectively for measuring immune stimulation in the form of antigen specific induction of T-cell proliferation. FIGS. 9e and 9f showed that more than 90% inhibition of antigen specific induction of T-cell proliferation is achieved using the human mAb's of the instant invention.

19. Selection of Useful Polypeptide for the Treatment of Cancers

In order to select the most appropriate protein/peptide to enter further experiments and to assess its suitability for use in a therapeutic composition for the treatment of cancers, additional data are collected. Such data for each IgG form of the anti-HLA antigen antibody fragments can include the binding affinity, in vitro killing efficiency as measured by EC₅₀ and cytotoxicity across a panel of tumor cell lines, the maximal percentage cell killing as estimated in vitro, and tumor reduction data and mouse survival data from in vivo animal models.

The IgG form of the anti-ALA antigen antibody fragments that shows the highest affinity, the lowest EC₅₀ for killing, the highest maximal percentage cell killing and broadest across various tumor cell lines, the best tumor reduction data and/or the best mouse-survival data may be chosen to enter further experiments. Such experiments may include, for example, therapeutic profiling and toxicology in animals and phase I clinical trials in humans.

20. In vivo Efficacy of Immunosuppression using an HL-DR Specific Antibody in Treating Delayed-Type-Hypersensitivity (DTH)

In order to determine the in vivo efficacy of the immunosuppression activity of the mAb's of the instant invention, we conducted experiments using a mouse model for delayed-type-hypersensitivity (DTH). In this system, mouse ear-swelling in response to treatments by haptens such as oxazalone (OXA) or dinitroflurobenzene (DNFB) were measured to determine the in vivo efficacy of the mAb's of the instant invention.

Specifically, 0.05 ml of 2% OXA or DNFB were applied to the bellies of treatment group mice on day 1 and 2. On day 5, different doses of test mAb's 1D09C3 or control treatments were administered i.v. After waiting for 4 or 8 hours, mice were challenged with 0.02 ml of 0.5% OXA or DNFB. Ear thickness was measured on day 6, 8, 9 and 12, and the results were presented in FIGS. 9g, 9h and 9i.

In FIG. 9g, DTH to OXA as measured by ear-thickness was blocked by roughly 75% if 1 mg or 0.5 mg of mAb was administered i.v., while 0.5 mg of mAb or less has no significant effect.

In FIG. 9h, the time course of inhibition, by human anti-DR mAb, of DTH to DNFB in DR-tg mice as measured by ear-thickness was presented. DTH was almost completely blocked (P<0.005) at 7^{th} hour after treatment with the mAb 1D09C3, followed by a 60% block (P<0.01) at 18^{th} hr and no effect at 4 hr. FIG. 9i showed a positive correlation between the dose of mAb (1D09C3) used at the 7^{th} hour and the effectiveness of the inhibition of DTH in DR-tg mice Both 1 mg and 0.5 mg of 1D09C3 significantly (P<0.005) inhibited DTH while lower doses have no effect.

These experiments demonstrates that mAb's of the instant invention is capable of specifically inhibiting the very part of the immune system responsible for the unwanted immune reaction. It is an inhibition of immune reaction rather than suppression of existing immune reactions. Since the mAb's of the instant invention are fully human antibodies, rather than murine mAb or humanized murine antibodies, they are expected to have very low immunogenicity in the host and a much longer half life. In addition, most mAb's of the instant invention also have very high affinity in the pico molar range. These mAb's shall prove to be useful for a variety of immune diseases such as DTH and Graft v. Host Disease (GVHD).

21. Selection of Useful Polypeptide for the Treatment of Diseases of the Immune System

In order to select the most appropriate protein/peptide to enter further experiments and to assess its suitability for use in a therapeutic composition for the treatment of diseases of the immune system, additional data are collected. Such data for each monovalent antibody fragment or IgG form of the anti-HLA antigen antibody fragments can include the affinity, reactivity, specificity, IC₅₀-values, for inhibition of IL-2 secretion and of T-cell proliferation, or in vitro killing efficiency as measured by EC₅₀ and the maximal percentage cell killing as estimated in vitro, and DR-transgenic models of transplant rejection and graft vs. host disease.

The antibody fragment or IgG form of the anti-HLA antigen antibody fragments that shows the lowest EC₅₀, highest affinity, highest killing, best specificity and/or greatest inhibition of T-cell proliferation or IL-2 secretion, and high efficacy in inhibiting transplant rejection and/or graft vs. host disease in appropriate models, might be chosen to enter further experiments. Such experiments may include, for example, therapeutic profiling and toxicology in animals and phase I clinical trials in humans.

22. In Vivo Efficacy of Treating Different Diseases Using an HLA-DR Specific Antibody

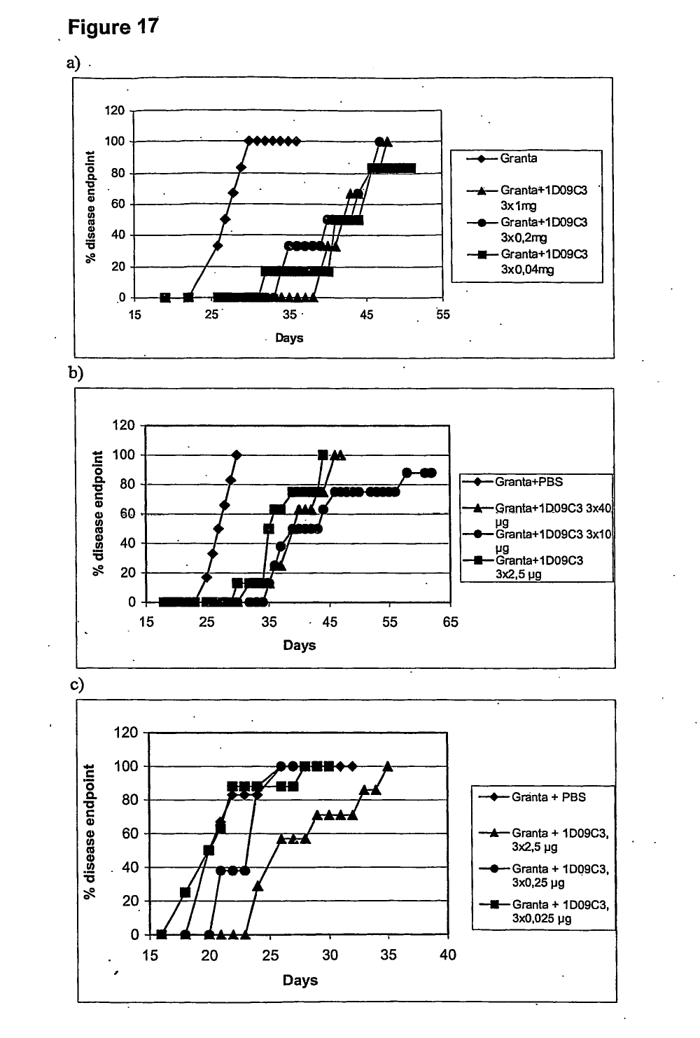

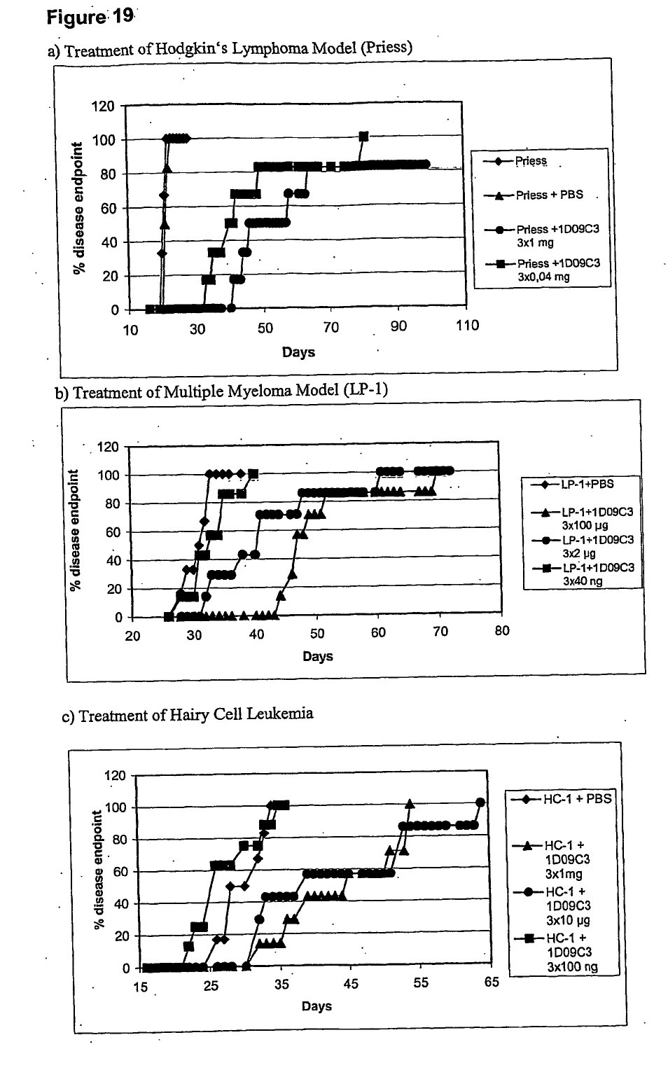

FIG. 17 shows that an HLA-DR specific antibody, the mAb 1D09C3, is effective n treating a Non-Hodgkin's Lymphoma model (Granta-519). FIG. 19 shows that 1D09C3 is effective in treating a Hodgkin's lymphoma model (Priess), a multiple myeloma model (LP-1) and a hariy cell leukemia model (HC-1).

To demonstrate the in vivo efficacy of the human antibody-based MHC II-binding antigen binding domain described herein (including 1D09C3) in xenotransplant models of Non-Hodgkin's Lymphoma, Hodgkin's lymphoma, multiple myeloma and hairy cell leukemia, immunocompromised SCID (severe combined immunodeficient) mice were intravenously (i.v.) inoculated with GRANTA-519 (DSMZ Accession No: ACC 342), Priess (ECACC Accession No: 86052111), LP-1 (DSMZ Accession No: ACC 41) or HC-1 (DSMZ Accession No: ACC 301) cells, and tumor development was monitored in those mice treated with the subject antibody compared to animals treated with solvent (PBS) alone.

Female C.B.-17 scid mice (8 weeks' old) were injected with anti-asialoGM1 antibody (Wako Chemicals, Neuss, Germany; 25 μl diluted 4 fold in PBS, i v.) to suppress natural killer (NK) cell activity, on days 0, 1, and 2. On day 1, 5×10⁶ GRANTA-519, Priess, LP-1 or HC-1 cells were injected i.v. The endpoint in the i.v. model is hind leg paralysis of grade 3 or larger or death.

Granta-519 (Non-Hodgkin's Lymphoma model): Mice were treated with 1 mg, 0.2 mg or 0.04 mg (FIG. 17 a; 6 mice/group), 40 μg, 10 μg or 2.5 μg (FIG. 17 b; 6 mice for the PBS control group, 8 mice for each antibody dose), or 2.5 μg, 0.25 μg or 0.025 μg (FIG. 17 c; 6 mice for the PBS control group, 7 mice for the 2.5 μg dose and 8 mice for each of the other two doses) 1D09C3 mAb i.v. on days 5, 7 and 9. The antibody exhibits comparable efficacy within a dose range of 1 mg to 2.5 μg per mouse (50 mg to 125 μg per kg). Efficacy titrates between 2.5 μg per mouse (full efficacy) and 25 ng per mouse (no detectable efficacy).

Priess (Hodgkin's Lymphoma model): Mice were treated with 1 mg or 0.04 mg 1D09C3 mAb i.v. on days 5, 6 and 7 (FIG. 19 a; 7 mice for the PBS control group, 6 mice for each antibody dose).

LP-1 (multiple myeloma model): Mice were treated with 100 μg, 2 μg or 40 ng 1D09C3 mAb i.v. on days 5, 9, 13 (FIG. 19 b, 6 mice for the PBS control group and the 100 μg dose, 7 mice for each of the other doses).

HC-1 (hariy cell leukemia model): Mice were treated with 1 mg, 10 μg or 100 ng 1D09C3 mAb i.v. on days 5, 7 and 9 (FIG. 19 c; 6 mice for the PBS control group, 7 mice for the 1 mg anf the 10 μg doses, 8 mice for the 100 ng dose).

23. Synergistism Between an HLA-DR Specific Antibody and the Anti-CD20 mAb Rituxan

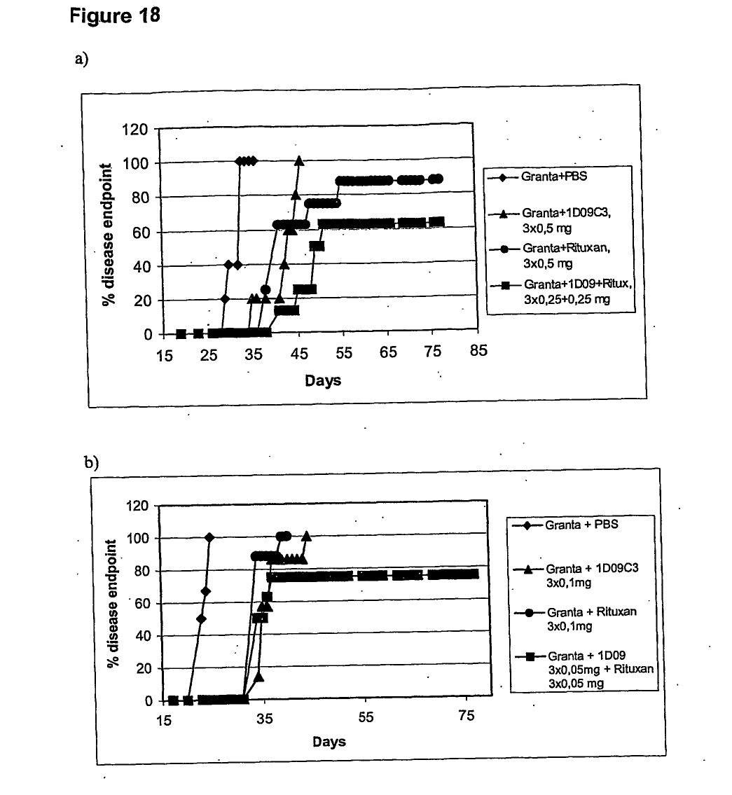

FIG. 18 shows that the mAb 1D09C3 and the anti-CD20 mAb Rituxan (Rituximab/MabThera) are synergistic in treating a Non-Hodgkin's Lymphoma model.

To demonstrate the in vivo efficacy and synergy of the human antibody-based MHC II-binding antigen binding domain described herein (including 1D09C3) when administered in combination with a second antibody-based antigen-binding domain that binds to a cell surface receptor (including Rituxan), immunocompromised (such as scid, nude or Rag-1 knockout) SCID (severe combined immunodeficient) mice were intravenously inoculated (i.v.) with GRANTA-519, and tumor development was monitored in those mice treated with the two antigen-binding domains in comparison to animals treated with each antigen-binding domain alone, and those treated with solvent alone.

Female C.B.-17 scid mice (8 weeks' old) were injected with anti-asialo GM1 antibody (Wako Chemicals, Neuss, Germany; 25 μl diluted 4 fold in PBS, i v.) to suppress natural killer (NK) cell activity, on days 0, 1, and 2. On day 1, 5×10⁶ GRANTA-519 cells were injected i.v. The endpoint in the i.v. model is hind leg paralysis of grade 3 or larger or death.

Mice were treated with 0.5 mg 1D09C3, 0.5 mg Rituxan or a mixture comprising 0.25 mg of each, 1D09C3 and Rituxan, i.v. on days 5, 7 and 9 (FIG. 18 a). In another experiment mice were treated with 0.1 mg 1D09C3, 0.1 mg Rituxan or a mixture comprising 0.05 mg of each, 1D09C3 and Rituxan, i.v. on days 5, 8 and 12 (FIG. 18 b). Five mice were used for the PBS control groups and the 1D09C3 single treatment groups. Eight mice were used for the Rituxan single treatment groups and the combination treatment groups.

Single therapies using each of these antibodies show comparable efficacies. The combined effects of the two antibodies are greater than the simple additive effects from single therapies using only one antibody, demonstrating synergism between the two antibodies. This finding is a first example of demonstrated synergism between a MHC class II molecule specific antibody and a cell surface receptor antibody such as the anti-CD20 antibody used here.

24. Killing of Melanoma Cell Lines by an HLA-DR Specific Antibody

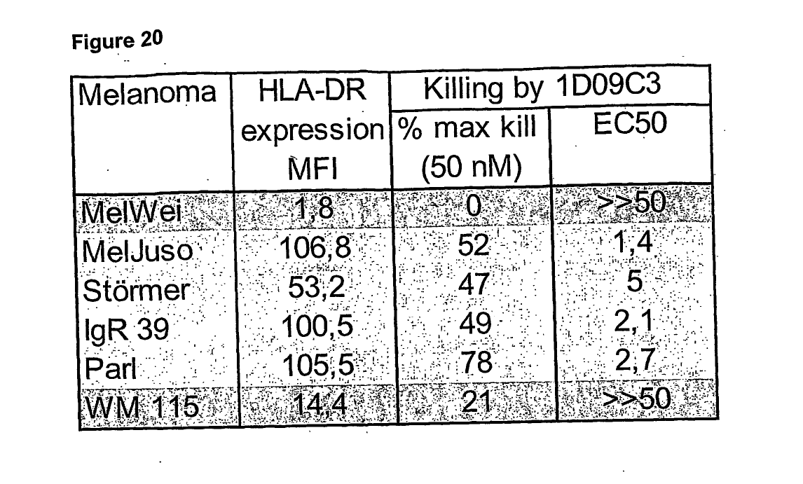

In addition to lymphoid tumor cells, a human MHC class II specific antibody, such as the 1D09C3 mAb, can also and surprisingly induce cell death in non-lympoid solid tumors, as evidenced by killing of HLA-DR+ melanoma cells in vitro. Cell lines used were MelWei, MelJuso (DSMZ Accession No: ACC 74), Störmer, IgR 39 (DSMZ Accession No: ACC 239), Parl and WM 115 (ECACC Accession No: 910612321. HLA-DR expression was measured by staining with the FITC-labelled antibody L243. MFI in FIG. 20 indicates the medium fluorescence intensity measured by FACS analysis.

For the measurement of cell killing cells were trypsinated using Trypsin EDTA in HBSS W/O Ca& Mg (Gibco BRL 25300-054, Life Technologies, Karlsruhe, Germany). Thereafter cells were stained with Trypan Blue using Trypan-blue Stain 0.4% (15250-061, Life Technologies, Karlsruhe, Germany). Viable cells were identified microscopically by exclusion of Trypan blue. Cell killing was quantified by counting viable and dead cells in a Neubauer chamber.

As summarized in FIG. 20, four out of the six melanoma cell lines, MelJuso, Störmer, IgR 39 and Parl, strongly express HLA-DR. Those four cell lines are effectively killed by the HLA-DR specific mAb 1D09C3. Cell lines MelWei and WM 115 showed hardly any or only weak expression of HLA-DR. No killing by 1D09C3 could be observed for MelWei, and only 21% of WM 115 cells were killed by 1D09C3.

Therefore, in addition to malignant lymphoid cells, 1D09C3 surprisingly can also induce cell death in non-lymphoid solid tumors cells. The 1D09C3 mAb exhibits comparable efficacy within a dose range of 1 mg to 2.5 μg/mouse (50 mg to 125 μg/kg).

25. Late Treatment of Disseminated Lymphoma with an HLA-DR Specific Antibody

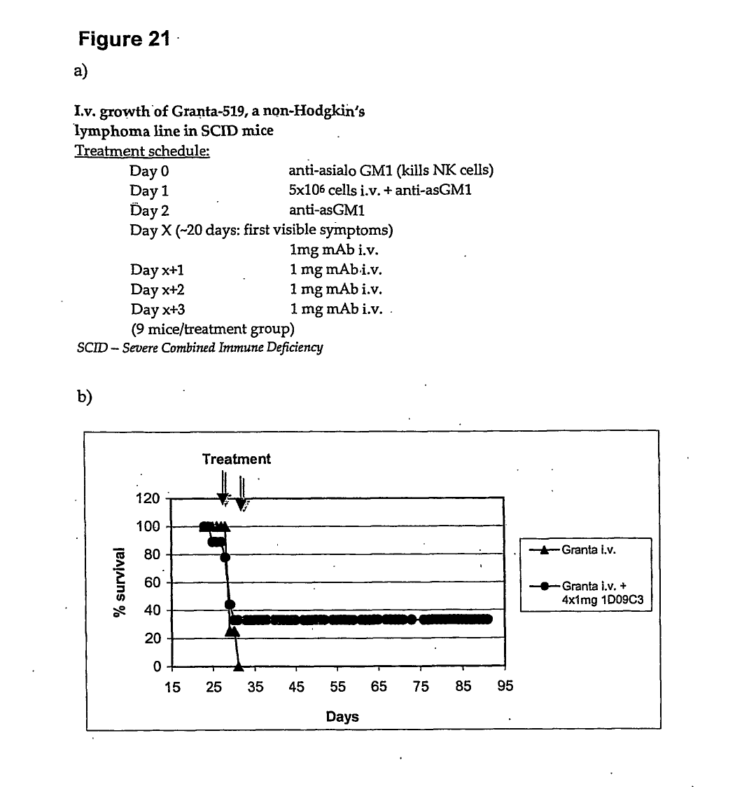

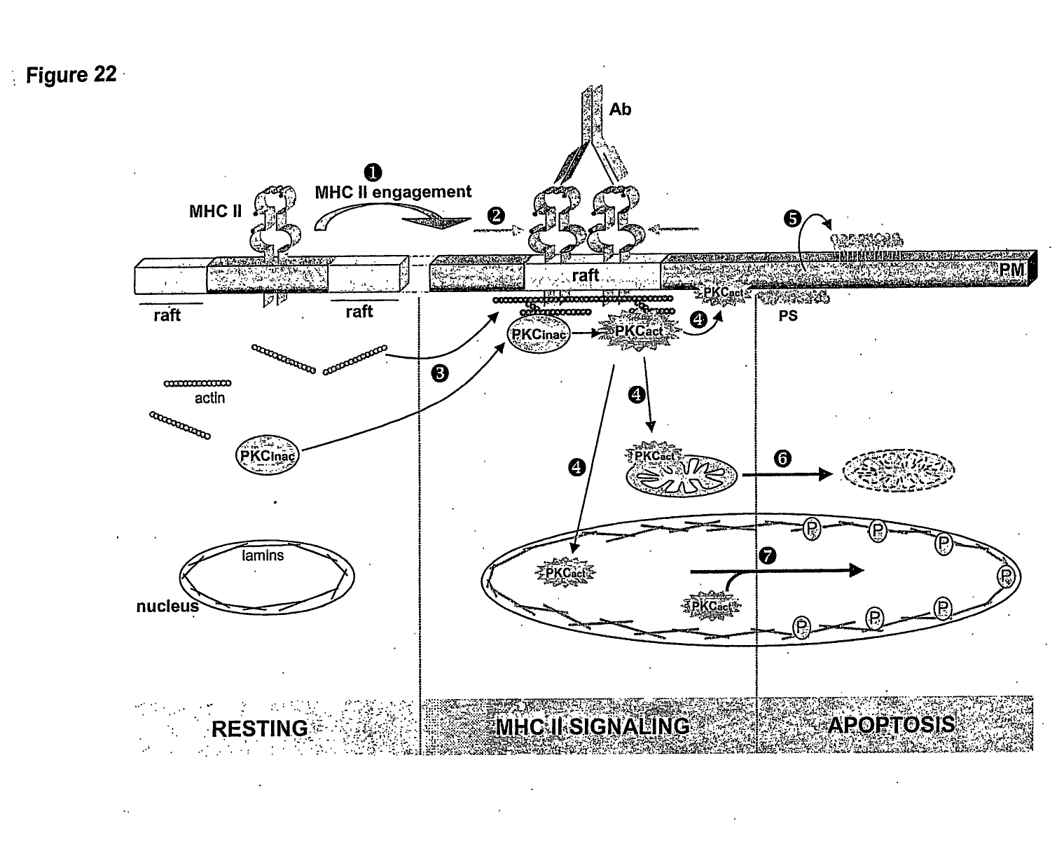

FIG. 21 shows that in a model of terminal stage disease (−7 days before moribund, histologically characterized as disseminated lymphoma in multiple organs), a human antibody-based antigen-binding domain, 1D09C3, could still rescue 33% of treated animals.

Female C.B.-17 scid mice (8 weeks' old) were injected with anti-asialoGM1 antibody (Wako Chemicals, Neuss, Germany; 25 μl diluted 4 fold in PBS, i v.) to suppress natural killer (NK) cell activity, on days 0, 1, and 2. On day 1, 5×10⁶ GRANTA-519 cells were injected i.v. Nine mice per group were used. As soon as a mouse developed symptoms, treatment was started comprising 1 mg of 1D09C3 daily on four consecutive days. The first symptom seen was usually a ruffling of fur. The first symptoms were not seen on the same day for each mouse, rather each mouse was individually examined, and as soon as the first symptom was seen, treatment was initiated (roughly around day 20).

As shown in FIG. 21 1D09C3 could rescue 33% (3 out of 9) of the treated animals. Of note, two out of the three rescued mice were tumor-free, even histologically. The third mouse rescued had one tumor only which was localized in the hip, i.e. there were no sign of any dissemination.

### EQUIVALENTS

Those skilled in the art will recognize, or be able to ascertain using no more than routine experimentation, many equivalents to the specific embodiments of the invention described herein. Such equivalents are intended to be encompassed by the following claims. Those skilled in the art will also recognize that all combinations of embodiments or features of the claims described herein are within the scope of the invention.

TABLE 1

VH and VL families, VL CDR1 and VH/VL CDR 3 sequences of HLA-DR-specific polypeptides

| | | | | | | | | |
|---|---|---|---|---|---|---|---|---|
| | | CDR3 | | | | CDR3 | | Fami- |
| Clone | VH | Length | VH-CDR3-Seq. | VL | VL-CDR1-Seq. | Length | VL-CDR3-Seq. | lies |

| | | | | | | | | |
|---|---|---|---|---|---|---|---|---|
| MS-GPC-1 | H2 | 10 | QYGHRGGFDH | λ 1 | SGSSSNIGSNYVS | 8 | QSYDFNES | H2 λ 1 |
| | | | (SEQ ID NO: 19) | | (SEQ ID NO: 12) | | (SEQ ID NO: 59) | |
| MS-GPC-6 | H3 | 9 | GYGRYSPDL | K3 | RASQSVSSSYLA | 8 | QQYSNLPF | H3 K 3 |
| | | | (SEQ ID NO: 20) | | (SEQ ID NO: 62) | | (SEQ ID NO: 21) | |
| MS-GPC-8 | H2 | 10 | SPRYRGAFDY | λ 1 | SGSSSNIGSNYVS | 8 | QSYDMPQA | H2 λ 1 |
| | | | (SEQ ID NO: 3) | | (SEQ ID NO: 12) | | (SEQ ID NO: 22) | |
| MS-GPC-10 | H2 | 10 | QLHYRGGFDL | λ 1 | SGSSSNIGSNYVS | 8 | QSYDLTMG | H2 λ 1 |
| | | | (SEQ ID NO: 61) | | (SEQ ID NO: 12) | | (SEQ ID NO: 23) | |
| MS-GPC-8-1 | H2 | 10 | SPRYRGAFDY | λ 1 | SGSSSNIGSNYVS | 8 | QSYDFSHY | H2 λ 1 |
| | | | (SEQ ID NO: 3) | | (SEQ ID NO: 12) | | (SEQ ID NO: 24) | |
| MS-GPC-8-6 | H2 | 10 | SPRYRGAFDY | λ 1 | SGSSSNIGSNYVS | 8 | QSYDYDHY | H2 λ 1 |
| | | | (SEQ ID NO: 3) | | (SEQ ID NO: 12) | | (SEQ ID NO: 60) | |
| MS-GPC-8-9 | H2 | 10 | SPRYRGAFDY | λ 1 | SGSSSNIGSNYVS | 8 | QSYDIQLH | H2 λ 1 |
| | | | (SEQ ID NO: 3) | | (SEQ ID NO: 12) | | (SEQ ID NO: 25) | |
| MS-GPC-8-10 | H2 | 10 | SPRYRGAFDY | λ 1 | SGSSSNIGSNYVS | 8 | QSYDLIRH | H2 λ 1 |
| | | | (SEQ ID NO: 3) | | (SEQ ID NO: 12) | | (SEQ ID NO: 4) | |
| MS-GPC-8-17 | H2 | 10 | SPRYRGAFDY | λ 1 | SGSSSNIGSNYVS | 8 | QSYDFSVY | H2 λ 1 |
| | | | (SEQ ID NO: 3) | | (SEQ ID NO: 12) | | (SEQ ID NO: 26) | |
| MS-GPC-8-18 | H2 | 10 | SPRYRGAFDY | λ 1 | SGSSSNIGSNYVS | 8 | QSYDFSIY | H2 λ 1 |
| | | | (SEQ ID NO: 3) | | (SEQ ID NO: 12) | | (SEQ ID NO: 27) | |
| MS-GPC-8-27 | H2 | 10 | SPRYRGAFDY | λ 1 | SGSSSNIGSNYVS | 8 | QSYDMNVH | H2 λ 1 |
| | | | (SEQ ID NO: 3) | | (SEQ ID NO: 12) | | (SEQ ID NO: 5) | |
| MS-GPC-8-6-2 | H2 | 10 | SPRYRGAFDY | λ 1 | SGSESNIGSNYVH | 8 | QSYDYDHY | H2 λ 1 |
| | | | (SEQ ID NO: 3) | | (SEQ ID NO: 13) | | (SEQ ID NO: 60) | |
| MS-GPC-8-6-19 | H2 | 10 | SPRYRGAFDY | λ 1 | SGSESNIGSNYVA | 8 | QSYDYDHY | H2 λ 1 |
| | | | (SEQ ID NO: 3) | | (SEQ ID NO: 14) | | (SEQ ID NO: 60) | |
| MS-GPC-8-6-27 | H2 | 10 | SPRYRGAFDY | λ 1 | SGSDSNIGANYVT | 8 | QSYDYDHY | H2 λ 1 |
| | | | (SEQ ID NO: 3) | | (SEQ ID NO: 15) | | (SEQ ID NO: 60) | |
| MS-GPC-8-6-45 | H2 | 10 | SPRYRGAFDY | λ 1 | SGSEPNIGSNYVF | 8 | QSYDYDHY | H2 λ 1 |
| | | | (SEQ ID NO: 3) | | (SEQ ID NO: 16) | | (SEQ ID NO: 60) | |
| MS-GPC-8-6-13 | H2 | 10 | SPRYRGAFDY | λ 1 | SGSESNIGANYVT | 8 | QSYDYDHY | H2 λ 1 |
| | | | (SEQ ID NO: 3) | | (SEQ ID NO: 29) | | (SEQ ID NO: 60) | |
| MS-GPC-8-6-47 | H2 | 10 | SPRYRGAFDY | λ 1 | SGSESNIGSNYVS | 8 | QSYDYDHY | H2 λ 1 |
| | | | (SEQ ID NO: 3) | | (SEQ ID NO: 30) | | (SEQ ID NO: 60) | |
| MS-GPC-8-10-57 | H2 | 10 | SPRYRGAFDY | λ 1 | SGSESNIGNNYVQ | 8 | QSYDLIRH | H2 λ 1 |
| | | | (SEQ ID NO: 3) | | (SEQ ID NO: 7) | | (SEQ ID NO: 4) | |
| MS-GPC-8-27-7 | H2 | 10 | SPRYRGAFDY | λ 1 | SGSESNIGNNYVG | 8 | QSYDMNVH | H2 λ 1 |
| | | | (SEQ ID NO: 3) | | (SEQ ID NO: 17) | | (SEQ ID NO: 5) | |
| MS-GPC-8-27-10 | H2 | 10 | SPRYRGAFDY | λ 1 | SGSESNIGANYVN | 8 | QSYDMNVH | H2 λ 1 |
| | | | (SEQ ID NO: 3) | | (SEQ ID NO: 18) | | (SEQ ID NO: 5) | |
| MS-GPC-8-27-41 | H2 | 10 | SPRYRGAFDY | λ 1 | SGSESNIGNNYVQ | 8 | QSYDMNVH | H2 λ 1 |
| | | | (SEQ ID NO: 3) | | (SEQ ID NO: 7) | | (SEQ ID NO: 5) | |

TABLE 2

| Steps in | | | | | | |
|---|---|---|---|---|---|---|
| Antibody | | kₒₙ [s⁻¹M⁻¹] × | k_{off} [s⁻¹] × | K_{D} | | |
| optimisation | Fab | 10⁵ ± SD | 10⁻³ ± SD | [nM] ± SD | L-CDR3 | L-CDR1 |
| Parental Fab | MS-GPC-8 | 0.99 ± 0.40 | 29.0 ± 8.40 | 346.1 ± 140.5^{a)} | QSYDMPQA | SGSSSNIGSNYVS |
| | | | | | (SEQ ID NO: 22) | (SEQ ID NO: 12) |
| L-CDR3-optim. | -8-1 | 1.93 | 20.9 | 108^{e)} | | |
| L-CDR3-optim. | -8-6 | 0.96 ± 0.14 | 5.48 ± 0.73 | 58.6 ± 11.7^{b)} | | |
| L-CDR3-optim. | -8-9 | 1.85 | 16.6 | 90.1 ^{e)} | | |
| L-CDR3-optim. | -8-10 | nd | 7.0^{e)} | nd | | |
| L-CDR3-optim. | -8-17 | 1.0 | 5.48 | 54.7 ^{e)} | | |
| L-CDR3-optim. | -8-18 | 1.06 | 8.3 | 78.3 ^{e)} | | |
| L-CDR3-optim. | -8-27 | nd | 6.6^{e)} | nd | | |
| L-CDR3-optim. | -8-6 | 0.96 ± 0.14 | 5.48 ± 0.73 | 58.6 ± 11.7^{b)} | QSYDYDHY | SGSSSNIGSNYVS |
| | | | | | (SEQ ID NO: 60) | (SEQ ID NO: 12) |
| L-CDR3 + 1-opt. | -8-6-2 | 1.23 ± 0.11 | 0.94 ± 0.07 | 7.61 ± 0.25^{c)} | QSYDYDHY | SGSESNIGSNYVH |
| | | | | | (SEQ ID NO: 60) | (SEQ ID NO: 13) |
| L-CDR3 + 1-opt. | -8-6-19 | 1.10 ± 0.08 | 0.96 ± 0.15 | 8.74 ± 1.33^{c)} | QSYDYDHY | SGSESNIGSNYVA |
| | | | | | (SEQ ID NO: 60) | (SEQ ID NO: 14) |
| L-CDR3 + 1-opt. | -8-6-27 | 1.80 ± 0.24 | 1.10 ± 0.15 | 6.30 ± 0.63^{d)} | QSYDYDHY | SGSDSNIGANYVT |
| | | | | | (SEQ ID NO: 60) | (SEQ ID NO: 15) |
| L-CDR3 + 1-opt. | -8-6-45 | 1.20 ± 0.07 | 1.03 ± 0.04 | 8.63 ± 0.61^{c)} | QSYDYDHY | SGSEPNIGSNYVF |
| | | | | | (SEQ ID NO: 60) | (SEQ ID NO: 16) |
| L-CDR3 + 1-opt. | -8-6-13 | 1.90 ± 0.26 | 0.55 ± 0.05 | 2.96 ± 0.46^{c)} | QSYDYDHY | SGSESNIGANYVT |
| | | | | | (SEQ ID NO: 60) | (SEQ ID NO: 29) |
| L-CDR3 + 1-opt. | -8-6-47 | 1.97 ± 0.29 | 0.62 ± 0.04 | 3.18 ± 0.33^{c)} | QSYDYDHY | SGSESNIGSNYVS |
| | | | | | (SEQ ID NO: 60) | (SEQ ID NO: 30) |
| L-CDR3 + 1-opt. | -8-10-57 | 1.65 ± 0.21 | 0.44 ± 0.06 | 2.67 ± 0.25^{c)} | QSYDLIRH | SGSESNIGNNYVQ |
| | | | | | (SEQ ID NO: 4) | (SEQ ID NO: 7) |
| L-CDR3 + 1-opt. | -8-27-7 | 1.74 ± 0.21 | 0.57 ± 0.07 | 3.30 ± 0.34^{d)} | QSYDMNVH | SGSESNIGNNYVG |
| | | | | | (SEQ ID NO: 5) | (SEQ ID NO: 17) |
| L-CDR3 + 1-opt. | -8-27-10 | 1.76 ± 0.21 | 0.53 ± 0.05 | 3.01 ± 0.21^{c)} | QSYDMNVH | SGSESNIGANYVN |
| | | | | | (SEQ ID NO: 5) | (SEQ ID NO: 18) |
| L-CDR3 + 1-opt. | -8-27-41 | 1.67 ± 0.16 | 0.49 ± 0.03 | 2.93 ± 0.27^{d)} | QSYDMNVH | SGSESNIGNNYVQ |
| | | | | | (SEQ ID NO: 5) | (SEQ ID NO: 7) |

^{a)}Affinity data of MS-GPC-8 are based on 8 different Fab-preparations which were measured on 4 different chips (2 × 500, 1000, 4000RU)
^{b)}For MS-GPC-8-6 mean and standard deviation of 3 different preparations on 3 different chips (500, 4000, 3000RU) is shown.
^{c)}3000RU MHCII were immobilized on a CM5-chip. For each measurement 7 different concentrations from 1 μM to 16 nM were injected on the surface. Dissociation time: 150 sec, regeneration was reached by 6 μl 10 mM Glycine pH 2.3 followed by 8 μl 7.5 mM NaOH. For MS-GPC-8-6-19 mean and standard deviation of 4 different preparations are shown whereas for all other binders mean and standard deviation of 3 different preparations are shown.
^{d)}One protein preparation is measured on 3 different chips (3000, 2800 and 6500RU).
^{e)} Affinity determination of maturated MHCII binder on a 4000RU density chips; single measurement.
Molecular weights were determined after size exclusion chromatography and found 100% monomeric with the right molecular weight between 45 and 48 kDa.

TABLE 3a
Affinities of selected IgG₄ monoclonal antibodies constructed from F_{ab}'s.
Errors represent standard deviations

| | Binder (IgG₄) | | |
|---|---|---|---|
| | kₒₙ [M⁻¹ s⁻¹] × 10⁵ | k_{off} [s⁻¹] × 10⁻⁵ | K_{D} [nM] |
| MS-GPC-8-27-41 | 1.1 ± 0.2 | 3.1 ± 0.4 | 0.31 ± 0.06 |
| MS-GPC-8-6-13 | 0.7 ± 0.1 | 3.0 ± 1.0 | 0.50 ± 0.20 |
| MS-GPC-8-10-57 | 0.7 ± 0.2 | 4.0 ± 1.0 | 0.60 ± 0.20 |

TABLE 3b
Affinities of binders obtained out of affinity maturation
of CDR1 light chain optimisation following CDR3 heavy chain
optimisation. Errors represent standard deviations

| | Binder (F_{ab}) | | |
|---|---|---|---|
| | kₒₙ [M⁻¹s⁻¹] × 10⁵ | k_{off} [s⁻¹] × 10⁻³ | K_{D} [nM] |
| MS-GPC-8-6-2 | 1.20 ± 0.10 | 0.94 ± 0.07 | 7.6 ± 0.3 |
| MS-GPC-8-6-19 | 1.10 ± 0.10 | 1.00 ± 0.20 | 9.0 ± 1.0 |
| MS-GPC-8-6-27 | 1.80 ± 0.20 | 1.10 ± 0.20 | 6.3 ± 0.6 |
| MS-GPC-8-6-45 | 1.20 ± 0.07 | 1.03 ± 0.04 | 8.6 ± 0.6 |
| MS-GPC-8-6-13 | 1.90 ± 0.30 | 0.55 ± 0.05 | 3.0 ± 0.5 |
| MS-GPC-8-6-47 | 2.00 ± 0.30 | 0.62 ± 0.04 | 3.2 ± 0.3 |
| MS-GPC-8-10-57 | 1.70 ± 0.20 | 0.44 ± 0.06 | 2.7 ± 0.3 |
| MS-GPC-8-27-7 | 1.70 ± 0.20 | 0.57 ± 0.07 | 3.3 ± 0.3 |
| MS-GPC-8-27-10 | 1.80 ± 0.20 | 0.53 ± 0.05 | 3.0 ± 0.2 |
| MS-GPC-8-27-41 | 1.70 ± 0.20 | 0.49 ± 0.03 | 2.9 ± 0.3 |

TABLE 3c
Binders obtained out of affinity maturation
of GPC8 by CDR3 light chain optimisation

| | Binder (F_{ab}) | | |
|---|---|---|---|
| | kₒₙ [M⁻¹s⁻¹] × 10⁵ | k_{off} [s⁻¹] × 10⁻³ | K_{D} [nM] |
| MS-GPC 8-18 | 1.06 | 8.30 | 78.3 |
| MS-GPC 8-9 | 1.85 | 16.60 | 90.1 |
| MS-GPC 8-1 | 1.93 | 20.90 | 108.0 |
| MS-GPC 8-17 | 1.00 | 5.48 | 54.7 |
| MS-GPC-8-6^{a)} | 1.20 ± 0.10 | 5.50 ± 0.70 | 8.0 ± 12.0 |

Chip density 4000RU MHCII
^{a)}For MS-GPC-8-6 mean and standard deviation of 3 different preparations on 3 different chips (500, 4000, 3000RU) is shown.

TABLE 3d
Binders obtained out of HuCAL in scFv form and their converted Fabs

| | Binder | | | | | |
|---|---|---|---|---|---|---|
| | scFᵥ | | | F_{ab} | | |
| | kₒₙ | k_{off} | | kₒₙ | k_{off} | |
| | [M⁻¹s⁻¹] × 10⁵ | [s⁻¹] × 10⁻³ | K_{D} [nM] | [M⁻¹s⁻¹] × 10⁵ | [s⁻¹] × 10⁻³ | K_{D} [nM] |
| MS-GPC 1 | 0.413 | 61 | 1500 | 0.639 | 53 | 820 |
| MS-GPC 6 | 0.435 | 200 | 4600 | 0.135 | 114 | 8470 (1 curve) |
| MS-GPC 8 | 0.114 | 76 | 560 | 0.99 +/− 0.40^{b)} | 29.0 +/− 8.4 | 346^{a)} +/− 141 |
| MS-GPC 10 | 0.187 | 180 | 9625 | 0.22 | 63 | 2860 |

Chip density 500RU MHCII
^{a)}Affinity data of MS-GPC-8 are based on 8 different Fab-preparations which were measured on 4 different chips (2 × 500, 1000, 4000RU) and are shown with standard deviation.
^{b)}Mean ± S.D. of three independent measurements.

TABLE 3e
Affinity improvements achieved by antibody optimization

| mAb | Format | Optimization | kₒₙ [s⁻¹M⁻¹] × 10⁵ | k_{off} [s⁻¹] × 10⁻³ | K_{D} [nM]^{a} |
|---|---|---|---|---|---|
| B8 | Fab | parental | 0.99 ± 0.4^{b} | 29.0 ± 8.4 | 346.1 ± 140.5 |
| 7BA | Fab | L-CDR3 | 0.96 ± 0.14 | 5.48 ± 0.73 | 58.6 ± 11.7 |
| 305D3 | Fab | L-CDR3 + 1 | 1.90 ± 0.26 | 0.55 ± 0.05 | 2.96 ± 0.46 |
| 1C7277 | Fab | L-CDR3 + 1 | 1.65 ± 0.21 | 0.44 ± 0.06 | 2.67 ± 0.25 |
| 1D09C3 | Fab | L-CDR3 + 1 | 1.67 ± 0.16 | 0.49 ± 0.03 | 2.93 ± 0.27 |
| 305D3 | IgG₄ | L-CDR3 + 1 | 0.71 ± 1.6 | 0.33 ± 1.0 | 0.5 ± 0.20 |
| 1C7277 | IgG₄ | L-CDR3 + 1 | 0.11 ± 2.0 | 0.31 ± 0.4 | 0.3 ± 0.06 |
| 1D09C3 | IgG₄ | L-CDR3 + 1 | 0.71 ± 1.2 | 0.41 ± 1.1 | 0.6 ± 0.20 |

^{a}Affinities were determined by BiaCore.
^{b}Mean ± S.D. of three independent measurements.

TABLE 4
Killing efficiency after 4 hour incubation of cells
with cross-linked anti-HLA-DR antibody fragments,
and maximum killing after 24 hour incubation

| Cross-linked | Killing efficiency against | Maximum killing against |
|---|---|---|
| Fab fragment | GRANTA | PRIESS |
| MS-GPC-1 | + | + |
| MS-GPC-6 | + | + |
| MS-GPC-8 | + | + |
| MS-GPC-10 | + | + |
| MS-GPC-8-6 | ++ | ++ |
| MS-GPC-8-17 | ++ | ++ |
| MS-GPC-8-6-13 | +++ | +++ |
| MS-GPC-8-10-57 | +++ | +++ |
| MS-GPC-8-27-41 | +++ | +++ |

TABLE 5
Killing efficiency of human anti-HLA-DR IgG antibodies compared to murine
anti-HLA-DR antibodies against a panel of lymphoid tumor cell lines.

| | | | HLA-DR | | | | | | |
|---|---|---|---|---|---|---|---|---|---|
| | | | expression^{a} | % Killing by mAb^{b} | | | | | |
| Cell Lines | | | MFL | Murine mAbs | | Human mAbs | | | |
| Name | Dr type | Tumor Type | L243 | L243 | 8D1 | B8 | 1D09C3 | 1C7277 | 305D3 |
| LG-2 | 1,1 | B-lymphoblastoid | 458 | 79 | 85 | 86 | 87 | 88 | 82 |
| PRIESS | 4,4 | B-lymphoblastoid | 621 | 87 | 83 | 85 | 88 | 93 | 74 |
| ARH-77 | 12 | B-lymphoblastoid | 301 | 88 | 73 | 84 | 85 | 88 | 87 |
| GRANTA-519 | 2,11 | B cell non-Hodgkin | 1465 | 83 | 56 | 76 | 78 | 78 | 73 |
| KARPAS-422 | 2,4 | B cell non-Hodgkin | 211 | 25 | 32 | 51 | 66 | 68 | 71 |
| KARPAS-299 | 1,2 | T cell non-Hodgkin | 798 | 78 | 25 | 81 | 82 | 79 | 76 |
| DOHH-2 | 1,2 | B cell lymphoma | 444 | 29 | 23 | 58 | 59 | 60 | 53 |
| SR-786 | 1,2 | T cell lymphoma | 142 | 3 | 8 | 1 | 53 | 44 | 26 |
| MHH-CALL-4 | 1,2 | B-ALL | 348 | 35 | 41 | 43 | 63 | 46 | 43 |
| MN-60 | 10,13 | B-ALL | 1120 | 46 | 22 | 71 | 69 | 66 | 67 |
| BJAB | 12,13 | Burkitt lymph. | 338 | 53 | 59 | 49 | 71 | 67 | 64 |
| RAJI | 10,17 | Burkitt lymph. | 617 | 69 | 64 | 81 | 84 | 86 | 83 |
| L-428 | 12 | Hodgkin's lymph. | 244 | 82 | 81 | 82 | 91 | 91 | 92 |
| HDLM-2 | | Hodgkin's lymph. | 326 | 77 | 73 | 89 | 88 | 84 | 90 |
| HD-MY-Z | | Hodgkin's lymph. | 79 | 35 | 39 | 49 | 69 | 57 | 72 |
| KM-H2 | | Hodgkin's lymph. | 619 | 81 | 56 | 75 | 86 | 88 | 87 |
| L1236 | | Hodgkin's lymph. | 41 | 52 | 62 | 44 | 63 | 66 | 66 |
| BONNA-12 | | hairy cell leuk. | 2431 | 92 | 91 | 91 | 92 | 91 | 86 |
| HC-1 | | hairy cell leuk. | 372 | 88 | 89 | 89 | 93 | 86 | 93 |
| NALM-1 | 1,4 | CML | 1078 | 44 | 4 | 83 | 82 | 78 | 65 |
| L-363 | | plasma cell leu. | 49 | 6 | 5 | 26 | 26 | 24 | 19 |
| EOL-1 | | AML (eosinophil) | 536 | 22 | 13 | 36 | 69 | 49 | 53 |
| LP-1 | | multiple myeloma | 315 | 12 | 0 | 61 | 73 | 70 | 73 |
| RPMI-8226 | | multiple myeloma | 19 | 6 | 0 | 14 | 29 | 26 | 19 |
| MHH-PREB-1 | | B cell non-Hodgkin | 175 | 3 | 3 | 2 | 4 | 8 | 11 |
| MHH-CALL-2 | | B cell precursor leu. | + | 5 | 5 | | | | |
| OPM-2 | | multiple myeloma | 3 | 13 | 0 | 8 | 1 | 4 | 5 |
| KASUMI-1 | | AML | 5 | 0 | 0 | 8 | 10 | 10 | 6 |
| HL-60 | | AML | 3 | 18 | 0 | 3 | 15 | 9 | 22 |
| LAMA-84 | | CML | 7 | 7 | 9 | 5 | 11 | 5 | 7 |

^{a}Expressed as mean fluorescence intensity after staning with FITC-labelled L243. Single determination or the average of 2 to 3 experiments per cell line.
^{b}Based on viable cell recovery after treatment with 200 nM murine or 50 nM human mAb at 37° C. for 4 h. Determined by light or fluorescence microscopic cell counting or FACS analysis, as described in Experimental protocol. Each number represents an average from 2 to 6 independent experiments.

TABLE 6
EC₅₀ values for certain anti-HLA-DR antibody fragments of
the invention in a cell-killing assay against lymphoid tumor
cells. All EC₅₀ refer to nanomolar concentrations of the
bivalent agent (IgG or cross-linked Fab) such that values
for cross-linked Fab and IgG forms can be compared.

| | | | EC₅₀ of cell killing |
|---|---|---|---|
| | | | (nM) +/− SE for |
| Antibody fragment | Form | Cell line tested | bivalent agent |
| MS-GPC-1 | Fab | PRIESS | 54 ± 14 |
| MS-GPC-8 | Fab | PRIESS | 31 ± 9 |
| MS-GPC-10 | Fab | PRIESS | 33 ± 5 |
| MS-GPC-8-17 | Fab | PRIESS | 16 ± 4 |
| MS-GPC-8-6-2 | Fab | PRIESS | 8 ± 2 |
| MS-GPC-8-10-57 | Fab | LG2 | 7.2 |
| MS-GPC-8-27-41 | Fab | LG2 | 7.2 |
| MS-GPC-8-27-41 | Fab | PRIESS | 7.7 |
| MS-GPC-8 | IgG₄ | PRIESS | 8.3 |
| MS-GPC-8-27-41 | IgG₄ | PRIESS | 1.1 ± 0.1 |
| MS-GPC-8-10-57 | IgG₄ | PRIESS | 1.1 ± 0.2 |
| MS-GPC-8-27-41 | IgG₄ | LG2 | 1.23 ± 0.2 |
| MS-GPC-8-10-57 | IgG₄ | LG2 | 1.0 ± 0.1 |
| 8D1 | mIgG | PRIESS | 33 |
| L243 | mIgG | PRIESS | 47 |

TABLE 7
IC₅₀ values for certain anti-HLA-DR antibody fragments of the
invention in an assay to determine IL-2 secretion after antigen-
specific stimulation of T-Hyb 1 cells. IC₅₀ for the IgG forms
(bivalent) are represented as molar concentrations, while in
order to provide easy comparison, IC₅₀s for the Fab forms
(monovalent) are expressed in terms of half the concentration
of the Fab to enable direct comparison to IgG forms.

| | | |
|---|---|---|
| | IC₅₀ | |
| | (IgG/nM) | |
| Anti-HLA-DR | (Fab)/2/nM) | Maximum |

| antibody fragment | Form | Mean | SE | inhibition(%) |
|---|---|---|---|---|
| MS-GPC-8-10-57 | IgG | 0.31 | 0.01 | 100 |
| MS-GPC-8-27-41 | IgG | 0.28 | 0.07 | 100 |
| MS-GPC-8-6-13 | IgG | 0.42 | 0.06 | 100 |
| MS-GPC-8-6-2 | IgG | 3.6 | 1.1 | 100 |
| MS-GPC-8-6 | IgG | 6.7 | 2.0 | 100 |
| MS-GPC-8 | IgG | 11.0 | 0.8 | 100 |
| MS-GPC-8-6-2 | Fab | 4.7 | 1.9 | 100 |
| MS-GPC-8-6-13 | Fab | 2.1 | 0.8 | 100 |
| MS-GPC-8-6-19 | Fab | 5.3 | 0.2 | 100 |
| MS-GPC-8-10-57 | Fab | 2.9 | 1.0 | 100 |
| MS-GPC-8-6-27 | Fab | 3.0 | 1.2 | 100 |
| MS-GPC-8-6-47 | Fab | 2.6 | 0.6 | 100 |
| MS-GPC-8-27-7 | Fab | 5.9 | 2.2 | 100 |
| MS-GPC-8-27-10 | Fab | 7.3 | 1.9 | 100 |
| MS-GPC-8-27-41 | Fab | 3.6 | 0.7 | 100 |
| MS-GPC-8-6 | Fab | 20 | | 100 |
| MS-GPC-8 | Fab | 110 | | 100 |

TABLE 8
Antibody Name Conversion Table

| | |
|---|---|
| MS-GPC-8 | B8 |
| MS-GPC-8-17 | 7BA |
| MS-GPC-8-6-13 | 305D3 |
| MS-GPC-8-10-57 | 1C7277 |
| MS-GPC-8-27-41 | 1D09C3 |
| MS-GPC-1 | 17 |
| MS-GPC-6 | 8A |
| MS-GPC-10 | E6 |

The following is a partial list of references cited in the instant application. The contacts of these references are hereby incorporated herein by reference.

### REFERENCES

Adorini L, Mueller S, Cardinaux F, Lehmann PV, Falcioni F, Nagy Z A, (1988), Nature 334: 623.
Ausubel, F. M., Brent, R., Kingston, R. E., Moore, D. D., Seidman, J. G., Smith, J. A. and Struhl, K. (1998) Current protocols in molecular biology. John Wiley & Sons, Inc., New York, U.S.A.
Babbitt B, Allen P M, Matsueda G, Habe E, Unanue ER, (1985), Nature 317:359.
Baxevanis, C. N., Wernet, D., Nagy, Z. A., Maurer, P. H., and Klein, J. (1980). Immunogenetics, 11, 617.
Billing, R., and Chatterjee, S. (1983). Transplant. Proc. 15, 649.
Bird, R. E. et al. Single-chain antigen-binding proteins [published erratum appears in Science 1989 Apr. 28; 244(4903)-409]. Science 242, 423-6 (1988).
Bonagura, V. R., Ma, a., McDowell, J., Lewison, A., King, D. W. and Suciu-Foca, N. (1987). Cell. Immunolo., 108(2), 356.
Brinkmann, U., Reiter, Y., Jung, S., Lee, B. & Pastan, I. (1993). A recombinant immunotoxin containing a disulfide-stabilized Fv fragment. Proc. Natl. Acad. Sci. U.S.A. 90, 7538-7542.
Brown J H, Jardetsky T S, Gorga J C, Stern L J, Urban R G, Strominger J L, Wiley D C., (1993), Nature 364: 33.
Buhmann R, Nolte A, Westhaus D, Emmerich B, Hallek M., (1999) Blood 93: 1992
Cambier J C, Morrison D C, Chien M M, Lehmann K R: J., (1991), Immunol. 146: 2075.
Current Protocols in Immunology, Vol. 2, 7.21 (1997).
Current Protocols in Immunology (John Wiley & Sons, Inc.; 1999).
Drenou B, Blancheteau V, Burgess DH, Fauchet R, Charron D J, Mooney N A., (1999), J. Immunol. 163: 4115.
Falcioni et al. (1999). Nat. Biotechnol. 17: 562-567.
Glockshuber, R., Malia, M., Pfitzinger, I & Pluckthun, A. (1990). A comparison of strategies to stabilize immunoglobulin Fv-fragments. Biochemistry 29, 1362-1367.
Gorga J. C., Foran, J., Burakoff, S. J., Strominger, J. L., (1984) Meth Emzym., 108, 607-613.
Gorga, J. C., Horejsi, V., Johnson, D. R., Raghupathy, R., Strominger, J. L., J. Biol. Chem., 262 (1987)16087-94.
Gorga, J. C., Knudsen, P. J., Foran, J. A., Strominger, J. L., Burakoff, S. J., (1986), Cell. Immunol. 103 160-73.
Heiskanen T, Lundkvist A, Soliymani R, Koivunen E, Vaheri A, Lankinen H (1999) Virology, 262(2), 321.
Hopp, T. P., Prickett, K. S., Price, V. L., Libby, R. T., March, C. J., Cerretti, D. P., Urdal, D. L. & Conlon, P. J. (1988), Bio/Technology 6, 1204-1210.
Huston, J. S. et al. Protein engineering of antibody binding sites: recovery of specific activity in an anti-digoxin single-chain Fv analogue produced in Escherichia coli. Proc Natl Acad Sci USA 85, 5879-83 (1988).
Ito K, Bian H.-J, Molina M, Han J, Magram J, Saar E, Belunis C, Bolin D R, Arceo R, Campbell R, Falcioni F, Vidovic' D, Nagy Z A., (1996), J. Exp. Med. 183; 2635-2644.
Jones et al., (1986), Nature 321: 522-525.
Jonker, M., Schellekens, P. T., Harpprecht, J., and Slingerland, W. (1991), Transplant. Proc., 23, 264.
Jonker, M., van Lambalgen, R., Mitchell, D. J., Durham, S. K., and Steinman, L. (1988), Autoimmunity, 1, 399.
Kabelitz D, Janssen O., (1989), Cell. Immunol. 120: 21.
Kashmiri S. V., Iwahashi, M., Tamura., Padlan, E. A., Milenic, D. E. & Sclom, J (2001) Crit Rev Oncol Hematol. 38: 3-16.
King, D. J., Turner, A., Farnsworth, A. P. H., Adair, J. R., Owens, R. J., Pedley, R. B., Baldock, D., Proudfoot, K. A., Lawson, A. D. G., Beeley, N. R. A., Millar, K., Millican, T. A., Boyce, B. A., Antoniw, P., Mountain, A., Begent, R. H. J., Shochat, D. and Yarranton, G. T., (1994), Cancer Res. 54, 6176.
Klohe EP, Watts R, Bahl M, Alber C, Yu W-Y, Anderson R, Silver J, Gregersen P K, Karr R K., (1988), J. Immunol. 141: 2158-2164.
Knappik, A. & Plückthun, A., (1994), Biotechniques 17, 754-761.
Knappik, A., Ge, L., Honegger, A., Pack, P., Fischer, M., Wellnhofer, G., Hoess A., Wölle, J., Plückthun, A. and Virnekäs, B., (2000), J. Mol. Biol. 296, 55.
Kahoury E. L. and Marshall L. A., (1990) Cell. Tissue Res., 262(2):217-24
Kozak, M. (1987) J. Mol. Biol. 196, 947.
Kuby, J. Immunology: 1994, 2^{nd} edition.
Lawson, Thomas, Roy, Gordon, Chawla, Nixon, Richmond, “Preparation of a monoclonal antibody to a melanoma growth-stimulatory activity released into serum-free culture medium by Hs0294 malignant melanoma cells,” J. Cell. Biochem., 34:169-185, 1987.
Mourad W, Geha R S, Chatila T J., (1990), J. Exp. Med. 172: 1513.
Muller et al., (1990), J. Immunol., 145: 4006.
Nabavi N, Freeman G J, Gault A, Godfrey D, Nadler L M, Glimcher L H., (1992) Nature 360: 266.
Nagy, Z & Vidovic, D. (1996) Monoclonal antibody fragments having immunosuppressant activity. WO9617874.
Nagy, Z., B Hubner, C Lohning, R Rauchenberger, S Reiffert, E Thomassen-Wolf, S Zahn, S Leyer, E Schier, A Zahradnik, C Brunner, K Lobenwein, B Rattel, M Stanglmaier, M Hallek, M Wing, S Anderson, M Dunn, T Kretzschmar, and M Tesar, Fully human, HLA-DR-specific monoclonal antibodies efficiently induce programmed death of malignant lymphoid cells, Nature Medicine Aug. 2002, Vol. 8, No. 7: 801-807
Nagy et al., U.S. patent application Ser. No. 10/001,934, filed Nov. 15, 2001, published on Feb. 13, 2002, as US-2003-0032782.
Nagy et al, PCT/US/01/15626, filed May 14, 2001, published on Nov. 22, 2001, as WO 01/87338.
Naquet, P., Marchetto, S., and Pierres, M., (1983), Immunogenetics, 18, 559.
Newell M K, VanderWall J, Beard K S, Freed J H., (1993), Proc. Natl. Acad. Sci. USA 90: 10459.
Otten et al (1997) pp 5.4.1-5.4.19 in Current Protocols in Immunology, Eds. Coligan et al. Green & Wiley, New York.
Pack, P. and Plückthun, A., (1992), Biochemistry 31, 1579-1584.
Pack, P., (1994), Ph.D. thesis, Ludwig-Maximilians-Universität München.
Pack, P., Kujau, M., Schroeckh, V., Knüpfer, U., Wenderoth, R., Riesenberg D. and Plückthun, A. (1993), Bio/Technology 11, 1271-1277.
Palacios R, Martinez-Maza O, Guy K., (1983), Proc. Natl. Acad. Sci. USA 80: 3456.
Palacios R., (1985), Proc. Natl. Acad. Sci. USA 82: 6652.
Presta, (1992), Curr. Op. Struct. Biol. 2: 593-596.
Pichla, S. L., Murali, R. & Burnett, R. M (1997) J Struct Biol. 119: 6-16.
Riechmann et al., (1988), Nature 332: 323-329.
Rheinnecker, M., Hardt, C., Ilag, L. L., Kufer, P., Gruber, R., Hoess, A., Lupas, A., Rottenberger, C., Plückthun, A. and Pack, P., (1996), J. Immunol. 157, 2989.
Rosenbaum J T, Adelman N E, McDevitt H O., (1981), J. Exp. Med. 154:1694.
Sambrook et al., 1989, Molecular Cloning: a Laboratory Manual, 2nd ed.
Schmidt, T. G. M. & Skerra, A. (1993). Prot. Engineering 6, 109-122.
Schmidt, T. G. M. & Skerra, A. (1994). J. Chromatogr. A 676, 337-345.
Schmidt, T. G. M. et al. (1996). J. Mol. Biol. 255, 753-766.
Singh, Gutman, Radinsky, Bucana, Fidler, “Expression of interleukin 8 correlates with metastatic potential of human melanoma cells in nude mice,” Cancer Research, 54:3242-3247, 1994.
Skerra, A. and Plückthun, A. (1988). Science 240, 1038.
Slavin-Chiorini, D. C., Kashmiri, S. V., Milenic, D. E., Poole, D. J., Bernono, E., Schlom, J. & Hand, P. H (1997) Cancer Biother Radiopharm 12: 305-316.
Smith, R. M., Morgan, A., and Wraith, D. C. (1994). Immunology, 83, 1.
Stausbøl-Grøn, B., Wind, T., Kjaer, S., Kahns, L., Hansen, N. J. V., Kristensen, P. and Clark, B. F. C. (1996) FEBS Lett. 391, 71.
Stern J. L. and Wiley, D. C., (1992), Cell 68 465-477.
Stern, A. S: and Podlaski, F. J, (1993) Techniques in Protein Chemistry IV, Academic Press Inc., San Diego, Calif.
Stevens, H. P., Roche, N., Hovius, S. E., and Jonker, M., (1990), Transplant. Proc., 22, 1783.
Truman J-P, Choqueux C, Tschopp J, Vedrenne J, Le Deist F, Charron D, Mooney N., (1997), Blood 89:1996.
Truman J-P, Ericson M L, Choqueux-Seebold J M, Charron D J, Mooney N A., (1994), Internatl. Immunol. 6: 887.
Vaickus L, Jones V E, Morton C L, Whitford K, Bacon R N., (1989), Cell. Immunol. 119: 445.
Vidovic D, Falcioni F, Bolin D R, Nagy Z A., (1995a), Eur. J. Immunol., 25: 1326.
Vidovic D, Falcioni F, Siklodi B, Belunis C J, Bolin D R, Ito K, Nagy Z A., (1995b), Eur J. Immunol., 25:3349.
Vidovic, D. & Laus, R. (2000) Selective apoptosis of neoplastic cells by the HLA-DR-specific monoclonal antibody. WO00/12560.
Vidovic D, & Toral, J. (1998). Selective apoptosis of neoplastic cells by the HLA-DR-specific monoclonal antibody. Cancer Letters 128: 127-135.
Virnekäs, B., Ge, L., Plukthun, A., Schneider, K. C., Wellenhofer, G. & Moroney, S. E. (1994) Nucleic Acids Res 22: 5600-5607.
Vode, J. M., Colcher, D., Gobar, L., Bierman, P. J., Augustine, S., Tempero, M., Leichner, P., Lynch, J. C., Goldenberg, D. & Armitage, J. O. (2000) Leuk Lymphoma 38: 91-101.
Voss, S. & Skerra, A. (1997). Protein Eng. 10, 975-982.
Waldor, M. K., Sriram, S., McDevitt, H. O., and Steinman, L. (1983). Proc. Natl. Acad. Sci. USA, 80, 2713.
Winter, G., Griffiths, A. D., Hawkins, R. E. and Hoogenboom, H. R. (1994) Making antibodies by phage display technology. Annu. Rev. Immunol. 12, 433.
Woods et al., (1994), J Exp Med. 180: 173-81.

1. A method of treating a disorder comprising administering to an individual in need thereof:
a. a first polypeptide comprising a human antibody-based antigen-binding domain that binds to a human class II MHC molecule; and
b. a second polypeptide comprising an antibody-based antigen-binding domain that binds to a cell surface receptor.

2. The method of claim 1, wherein said first and second polypeptides are administered concurrently.

3. The method of claim 1, wherein said first and second polypeptides are administered sequentially.

4. A method of treating a solid tumor comprising administering to an individual in need thereof a first polypeptide comprising a human antibody-based antigen-binding domain which binds to a human class II MHC molecule.

5. The method of claim 1 or 4, wherein said first polypeptide is further characterised in that treating cells expressing human class II MHC molecules with a multivalent first polypeptide having two or more of said antigen binding domains causes or leads to killing of said cells in a manner where neither cytotoxic entities nor immunological mechanisms are needed for said killing.

6. The method of claim 1 or 4, wherein said first polypeptide is part of a multivalent polypeptide that binds to a human class II MHC molecule.

7. The method of claim 1 or 4, wherein said first polypeptide is an antibody that binds to a human class II MHC molecule.

8. The method of claim 1 or 4, wherein said first polypeptide is a human monoclonal antibody that binds to a human class II MHC molecule.

9. The method of claim 1 or 4, wherein said first polypeptide binds to a human HLA-DR molecule.

10. The method of claim 1 or 4, wherein said first polypeptide is operably linked to a cytotoxic or immunogenic agent.

11. The method of claim 9, wherein said first polypeptide binds to one or more HLA-DR types selected from DR1-0101, DR2-15021, DR3-0301, DR4Dw4-0401, DR4Dw10-0402, DR4Dw14-0404, DR6-1302, DR6-1401, DR8-8031, DR9-9012, DRw53-B4*0101 and DRw52-B3*0101.

12. The method of claim 11, wherein said first polypeptide binds to at least 5 different HLA-DR types selected from DR1-0101, DR2-15021, DR3-0301, DR4Dw4-0401, DR4Dw10-0402, DR4Dw110404, DR6-1302, DR6-1401, DR8-8031, DR9-9012, DRw53-B4*0101 and DRw52-B3*010.

13. The method of claim 1 or 4, wherein said first polypeptide includes a combination of a VH domain and a VL domain, wherein said combination is found in one of the clones MS-GPC-1, MS-GPC-6, MS-GPC-8, MS-GPC-10, MS-GPC-8-1, MS-GPC-8-6, MS-GP C-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-647, MS-GPC-8-10-57, MS-GPC-8-27-7, MS-GPC-8-27-10 and MS-GPC-8-27-41.

14. The method of claim 1 or 4, wherein said first polypeptide includes of a combination of HuCAL VH2 and HuCAL Vλ1, wherein the VH CDR3, VL CDR1 And VL CDR3 is found in one of the clones MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-645, MS-GPC-8-6-13, MS-GPC-8-6-47, MS-GPC-8-10-57, MS-GPC-8-27-7, MS-GPC-8-27-10 and MS-GPC-8-27-41.

15. The method of claim 1 or 4, wherein said antigen-binding domain of the first polypeptide includes a combination of HuCAL VH2 and HuCAL Vλ1, wherein the VH CDR3 sequence is taken from the consensus CDR3 sequence
XXXXRGXFDX
wherein each X independently represents any amino acid residue; and/or
wherein the VL CDR3 sequence is taken from the consensus CDR3 sequence
QSYDXXXX
wherein each X independently represents any amino acid residue.

16. The method of claim 15 wherein the VH CDR3 sequence of said antigen-binding domain is SPRYRGAFDY and/or the VL CDR3 sequence of said antigen-binding domain is QSYDLIRH or QSYDMNVH.

17. The method of claim 1 or 4, wherein said first polypeptide competes for antigen binding with an antibody including a combination of HuCAL VH2 and HuCAL Vλ1, wherein the VH CDR3 sequence is taken from the consensus CDR3 sequence
XXXXRGXFDX
each X independently represents any amino acid residue; and/or
the VL CDR3 sequence is taken from the consensus CDR3 sequence
QSYDXXXX
each X independently represents any amino acid residue.

18. The method of claim 17, wherein the VH CDR3 sequence of said antibody is SPRYRGAFDY and/or the VL CDR3 sequence of said antibody is QSYDLIRH or QSYDMNVH.

19. The method of claim 1 or 4, wherein said first polypeptide includes a VL CDR1 sequence represented in the general formula
SGSXXNIGXNYVX
wherein each X independently represents any amino acid residue.

20. The method of claim 19, wherein the CDR1 sequence is SGSESNIGNNYVQ.

21. The method of claim 8, wherein said human monoclonal antibody is an IgG antibody obtainable by cloning into an immunoglobulin expression system an antigen-binding domain which includes a combination of a VH and a VL domain, wherein said combination is found in one of the clones MS-GPC-8-6-13, MS-GPC-8-10-57 or MS-GPC-8-27-41.

22. The method of claim 21, wherein said IgG antibody is an IgG4 antibody.

23. The method of claim 1, wherein said second polypeptide is operably linked to a cytotoxic or immunogenic agent.

24. The method of claim 1, wherein said second polypeptide binds to a cell surface receptor on a lymphocyte.

25. The method of claim 24, wherein said lymphocyte is a B cell.

26. The method of claim 1, wherein said second polypeptide comprises an antibody-based antigen-binding domain which binds to a cell surface receptor on a solid tumor cell.

27. The method of claim 1, wherein said second polypeptide comprises an antibody-based antigen-binding domain which binds to CD20.

28. The method of claim 1, wherein the second polypeptide comprises an anti-CD20 antibody.

29. The method of claim 28, wherein the anti-CD20 antibody is a monoclonal antibody.

30. The method of claim 29, wherein the anti-CD20 antibody is rituximab (RITUXAN®).

31. The method of claim 3, wherein the first and the second polypeptide are sequentially administered within a time period selected from about: 24 hours, 3 days, and, 7 days of each other.

32. A composition including a first polypeptide including a human antibody-based antigen-binding domain which binds to a human class II MHC molecule, and a second polypeptide comprising an antibody-based antigen-binding domain which binds to a cell surface receptor.

33. The composition of claim 32 further including a pharmaceutically acceptable carrier.

34. A pharmaceutical preparation including the composition of claim 31 or 32, for treating a disorder.

35. A pharmaceutical package for treating an individual suffering from a disorder, wherein said package includes a first polypeptide comprising a human antibody-based antigen-binding domain which binds to a human class II MHC molecule, and a second polypeptide comprising an antibody-based antigen-binding domain which binds to a cell surface receptor.

36. The pharmaceutical package of claim 35, wherein said first and second polypeptide are formulated separately.

37. The pharmaceutical package of claim 35, wherein said first and second polypeptide are formulated together.

38. The pharmaceutical package of claim 35, further comprising instructions to treat said disorder.

39. Use of a first polypeptide comprising a human antibody-based antigen-binding domain which binds to a human class II MHC molecule for the preparation of a pharmaceutical for the treatment of a disorder amenable to administration with said first polypeptide, wherein said first polypeptide is administered with a second polypeptide comprising an antibody-based antigen-binding domain which binds to a cell surface receptor.

40. Use of a second polypeptide comprising an antibody-based antigen-binding domain which binds to a cell surface receptor for the preparation of a pharmaceutical for the treatment of a disorder amenable to administration with said second polypeptide, wherein said second polypeptide is administered with a first polypeptide comprising a human antibody-based antigen-binding domain which binds to a human class II MHC molecule.

41. Use of (i) a first polypeptide comprising a human antibody-based antigen-binding domain which binds to a human class II MHC molecule for the preparation of a first pharmaceutical, and (ii) a second polypeptide comprising an antibody-based antigen-binding domain which binds to a cell surface receptor for the preparation of a second pharmaceutical, for the treatment of a disorder amenable to administration with said first and/or second polypeptides.

42. Use of (i) a first polypeptide comprising a human antibody-based antigen-binding domain which binds to a human class II MHC molecule, and (ii) a second polypeptide comprising an antibody-based antigen-binding domain which binds to a cell surface receptor, for the preparation of a pharmaceutical including both polypeptides for the treatment of a disorder amenable to administration with said first and/or second polypeptides.

43. The use of any one of claims 39 to 42, wherein said first and second polypeptides are administered concurrently.

44. The use of any one of claims 39 to 41, wherein said first and second polypeptides are administered sequentially.

45. Use of a first polypeptide comprising a human antibody-based antigen-binding domain which binds to a human class II MHC molecule for the preparation of a pharmaceutical for the treatment of solid tumors.

46. The method of claim 1, wherein said disorder is a cell proliferative disorder, is caused or contributed to by transformed cells expressing MHC class II antigens, is caused or contributed to by unwanted activation of cells of the immune system, such as lymphoid cells expressing MHC class II, or is caused or contributed to by non-lymphoid cells that express MHC class II molecules.

47. The method of claim 1, wherein said disorder is B cell non-Hodgkins lymphoma, B cell lymphoma, B cell acute lymphoid leukemia, Burkitt lymphoma, Hodgkins lymphoma, hairy cell leukemia, acute myeloid leukemia, T cell lymphoma, T cell non-Hodgkins lymphoma, chronic myeloid leukemia, chronic lymphoid leukemia, multiple myeloma, or multiple myeloid leukemia.

48. The method of claim 1 or 4, wherein said disorder or said solid tumor is adrenocortical carcinoma, carcinoma, colorectal carcinoma, desmoid tumor, desmoplastic small round cell tumor, endocrine tumor, Ewing sarcoma family tumors, germ cell tumors, hepatoblastoma, hepatocellular carcinoma, neuroblastoma, non-rhabdomyosarcoma soft tissue sarcoma, osteosarcoma, peripheral primitive neuroectodermal tumor, retinoblastoma, rhabdomyosarcoma or Wilms tumor.

49. The method of claim 1 or 4, wherein said disorder is a melanoma.

50. The method of claim 1, wherein said disorder is rheumatoid arthritis, juvenile arthritis, multiple sclerosis, Grave's disease, insulin-dependent diabetes, narcolepsy, psoriasis, systemic lupus erythematosus, ankylosing spondylitis, transplant rejection, graft vs. host disease, Hashimoto's disease, myasthenia gravis, pemphigus vulgaris, glomerulonephritis, thyroiditis, pancreatitis, insulitis, primary biliary cirrhosis, irritable bowel disease or Sjogren syndrome.

51. A method of treating a disorder comprising administering to an individual in need thereof:
a. a first polypeptide comprising an antibody-based antigen-binding domain selected from: MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-86-2, MS-GPC-8-6-19, MS-GPC-8-6-27, MS-GPC-8-6-45, MS-GPC-8-6-13, MS-GPC-8-647, MS-GPC-8-10-57, MS-GPC-8-27-7, MS-GPC-8-27-10, MS-GPC-8-27-41, a variant thereof or a modified version of the forgoing; and
b. a second polypeptide comprising rituximab (RITUXAN®).

52. The method of claim 51, wherein said first and second polypeptides are administered concurrently.

53. The method of claim 51, wherein said first and second polypeptides are administered sequentially.

54. Use of a first polypeptide comprising an antibody-based antigen-binding domain selected from: MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-6-45, MS-GPC-8-6-13, MS-GPC-8-6-47, MS-GPC-8-10-57, MS-GPC-8-27-7, MS-GPC-8-27-10, MS-GPC-8-27-41, a variant thereof or a modified version of the forgoing, for the preparation of a pharmaceutical for the treatment of a disorder amenable to administration with said first polypeptide, wherein said first polypeptide is administered with a second polypeptide comprising rituximab (RITUXAN®).

55. The use of any according to claim 54, wherein said first and second polypeptides are administered concurrently.

56. The use of any according to claim 54, wherein said first and second polypeptides are administered sequentially.

57. A method of treating a solid tumour comprising administering to an individual in need thereof a polypeptide comprising an antibody-based antigen-binding domain selected from: MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-645, MS-GPC-8-6-13, MS-GPC-8-647, MS-GPC-8-10-57, MS-GPC-8-27-7, MS-GPC-8-27-10, MS-GPC-8-27-41, a variant thereof or a modified version of the forgoing.

58. A method of treating a melanoma comprising administering to an individual in need thereof a polypeptide comprising an antibody-based antigen-binding domain selected from: MS-GPC-1, MS-GPC-8, MS-GPC-10, MS-GPC-8-1, MS-GPC-8-6, MS-GPC-8-9, MS-GPC-8-10, MS-GPC-8-17, MS-GPC-8-18, MS-GPC-8-27, MS-GPC-8-6-2, MS-GPC-8-6-19, MS-GPC-8-6-27, MS-GPC-8-645, MS-GPC-8-6-13, MS-GPC-8-647, MS-GPC-8-10-57, MS-GPC-8-27-7, MS-GPC-8-27-10, MS-GPC-8-27-41, a variant thereof or a modified version of the forgoing.

59. A method of killing or inhibiting the growth of a cell comprising contacting said cell with:
a. a first polypeptide comprising a human antibody-based antigen-binding domain which binds to a human class II MHC molecule; and
b. a second polypeptide comprising an antibody-based antigen-binding domain which binds to a cell surface receptor.

60. The method of claim 59, wherein said first and second polypeptides are contacted with said cell concurrently.

61. The method of claim 59, wherein said first and second polypeptides are contacted with said cell sequentially.

62. The method of any one of claims 59-61, wherein said cell is derived from or included in a tumour selected from: B cell non-Hodgkins lymphoma, B cell lymphoma, B cell acute lymphoid leukemia, Burkitt lymphoma, Hodgkins lymphoma, hairy cell leukemia, acute myeloid leukemia, T cell lymphoma, T cell non-Hodgkins lymphoma, chronic myeloid leukemia, chronic lymphoid leukemia, multiple myeloma, and multiple myeloid leukemia.

63. A method of killing or inhibiting the growth of a cell derived from or included in a solid tumour comprising contacting said cell with a first polypeptide comprising a human antibody-based antigen-binding domain which binds to a human class II MHC molecule.

64. The method of claim 63, wherein: said cell is derived from or included in a tumour selected from adrenocortical carcinoma, carcinoma, colorectal carcinoma, desmoid tumor, desmoplastic small round cell tumor, endocrine tumor, Ewing sarcoma family tumors, germ cell tumors, hepatoblastoma, hepatocellular carcinoma, neuroblastoma, non-rhabdomyosarcoma soft tissue sarcoma, osteosarcoma, peripheral primitive neuroectodermal tumor, retinoblastoma, rhabdomyosarcoma and Wilms tumor.

65. The method of claim 63 wherein said cell is derived from or included in a melanoma.
